SEQUENCE LISTING (75 sequences)

SEQ ID NO: 1 - skipped

SEQ ID NO: 2 - protein, 7 aa, synthetic construct
KKKKGGG

SEQ ID NO: 3 - skipped

SEQ ID NO: 4 - protein, 6 aa, synthetic construct
  VARIANT 1..2: note = 2-(2-(aminoethoxy)ethoxy]acetic acid
XXKKKK

SEQ ID NO: 5-22 - skipped (18 entries)

SEQ ID NO: 23 - protein, 29 aa, synthetic construct
  DISULFID 13..29: note = intrachain disulfide bond
KKKKGGGGLSKGCFGLKLDRIGSQSGLGC

SEQ ID NO: 24 - protein, 29 aa, synthetic construct
  DISULFID 13..29: note = intrachain disulfide bond
KKGGGKKGLSKGCFGLKLDRIGSQSGLGC

SEQ ID NO: 25 - protein, 29 aa, synthetic construct
  DISULFID 13..29: note = intrachain disulfide bond
GGGKKKKGLSKGCFGLKLDRIGSQSGLGC

SEQ ID NO: 26 - protein, 29 aa, synthetic construct
  DISULFID 13..29: note = intrachain disulfide bond
KKKKGGGGLSKGCFGLKLDRIGSQSGLGC

SEQ ID NO: 27 - protein, 29 aa, synthetic construct
  DISULFID 13..29: note = intrachain disulfide bond
KKGGGRRGLSKGCFGLKLDRIGSQSGLGC

SEQ ID NO: 28 - protein, 29 aa, synthetic construct
  VARIANT 6..7: note = diamino butanoic acid
  DISULFID 13..29: note = intrachain disulfide bond
KKGGGXXGLSKGCFGLKLDRIGSQSGLGC

SEQ ID NO: 29 - protein, 27 aa, synthetic construct
  VARIANT 1..3: note = 2-aminoethoxy-2-ethoxy acetic acid
  VARIANT 4..5: note = diamino butanoic acid
  DISULFID 11..27: note = intrachain disulfide bond
XXXXXGLSKGCFGLKLDRIGSQSGLGC

SEQ ID NO: 30 - protein, 27 aa, synthetic construct
  VARIANT 1: note = gamma linked glutamate
  VARIANT 2..3: note = 2-aminoethoxy-2-ethoxy acetic acid
  VARIANT 4..5: note = diamino butanoic acid
  DISULFID 11..27: note = intrachain disulfide bond
XXXXXGLSKGCFGLKLDRIGSQSGLGC

SEQ ID NO: 31 - protein, 27 aa, synthetic construct
  VARIANT 1..3: note = 2-aminoethoxy-2-ethoxy acetic acid
  VARIANT 4..5: note = diamino propionic acid
  DISULFID 11..27: note = intrachain disulfide bond
XXXXXGLSKGCFGLKLDRIGSQSGLGC

SEQ ID NO: 32 - protein, 22 aa, Homo sapiens
  REGION 1..22: note = native human C-type natriuretic peptide
GLSKGCFGLKLDRIGSMSGLGC

SEQ ID NO: 33 - protein, 27 aa, synthetic construct
  VARIANT 1..3: note = 2-aminoethoxy-2-ethoxy acetic acid
  VARIANT 4..5: note = diamino butanoic acid
  DISULFID 11..27: note = intrachain disulfide bond
XXXXXGLSKGCFGLKLDRIGSQSGLGC

SEQ ID NO: 34 - protein, 26 aa, synthetic construct
  VARIANT 1..2: note = 2-aminoethoxy-2-ethoxy acetic acid
  VARIANT 3..4: note = diamino butanoic acid
  DISULFID 10..26: note = intrachain disulfide bond
XXXXGLSKGCFGLKLDRIGSQSGLGC

SEQ ID NO: 35 - protein, 26 aa, synthetic construct
  VARIANT 1..2: note = 2-aminoethoxy-2-ethoxy acetic acid
  VARIANT 3: note = diamino propionic acid
  VARIANT 4: note = diamino butanoic acid
  DISULFID 10..26: note = intrachain disulfide bond
XXXXGLSKGCFGLKLDRIGSQSGLGC

SEQ ID NO: 36 - protein, 27 aa, synthetic construct
  VARIANT 1..3: note = gamma amino butyric acid
  VARIANT 4..5: note = diamino butanoic acid
  DISULFID 11..27: note = intrachain disulfide bond
XXXXXGLSKGCFGLKLDRIGSQSGLGC

SEQ ID NO: 37 - protein, 26 aa, synthetic construct
  VARIANT 1..2: note = gamma amino butyric acid
  VARIANT 3..4: note = diamino butanoic acid
  DISULFID 10..26: note = intrachain disulfide bond
XXXXGLSKGCFGLKLDRIGSQSGLGC

SEQ ID NO: 38 - protein, 27 aa, synthetic construct
  VARIANT 2..3: note = 2-aminoethoxy-2-ethoxy acetic acid
  VARIANT 4..5: note = diamino butanoic acid
  DISULFID 11..27: note = intrachain disulfide bond
EXXXXGLSKGCFGLKLDRIGSQSGLGC

SEQ ID NO: 39 - protein, 27 aa, synthetic construct
  VARIANT 1..3: note = 2-aminoethoxy-2-ethoxy acetic acid
  VARIANT 4..5: note = diamino propionic acid
  DISULFID 11..27: note = intrachain disulfide bond
XXXXXGLSKGCFGLKLDRIGSQSGLGC

SEQ ID NO: 40 - protein, 27 aa, synthetic construct
  VARIANT 1..3: note = 2-aminoethoxy-2-ethoxy acetic acid
  VARIANT 4..5: note = diamino propionic acid
XXXXXGLSKGCFGLKLDRIGSMSGLGC

SEQ ID NO: 41 - protein, 26 aa, synthetic construct
  VARIANT 1..3: note = 2-aminoethoxy-2-ethoxy acetic acid
  VARIANT 4: note = diamino butanoic acid
  DISULFID 10..26: note = intrachain disulfide bond
XXXXGLSKGCFGLKLDRIGSQSGLGC

SEQ ID NO: 42 - protein, 26 aa, synthetic construct
  VARIANT 1..3: note = 2-aminoethoxy-2-ethoxy acetic acid
  VARIANT 4: note = diamino propionic acid
  DISULFID 10..26: note = intrachain disulfide bond
XXXXGLSKGCFGLKLDRIGSQSGLGC

SEQ ID NO: 43 - protein, 28 aa, synthetic construct
  VARIANT 1..3: note = 2-aminoethoxy-2-ethoxy acetic acid
  VARIANT 4..6: note = diamino propionic acid
  DISULFID 12..28: note = intrachain disulfide bond
XXXXXXGLSKGCFGLKLDRIGSQSGLGC

SEQ ID NO: 44 - protein, 28 aa, Homo sapiens
  REGION 1..28: note = native human Atrial natriuretic peptide
SLRRSSCFGGRMDRIGAQSGLGCNSFRY

SEQ ID NO: 45 - protein, 33 aa, synthetic construct
  VARIANT 1..3: note = 2-aminoethoxy-2-ethoxy acetic acid
  VARIANT 4..5: note = diamino propionic acid
  DISULFID 12..28: note = intrachain disulfide bond
XXXXXSLRRSSCFGGRLDRIGAQSGLGCNSFRY

SEQ ID NO: 46 - protein, 33 aa, synthetic construct
  VARIANT 1..3: note = 2-aminoethoxy-2-ethoxy acetic acid
  VARIANT 4..5: note = diamino propionic acid
  DISULFID 12..28: note = intrachain disulfide bond
XXXXXSLRRSSCFGGRQDRIGAQSGLGCNSFRY

SEQ ID NO: 47 - protein, 33 aa, synthetic construct
  VARIANT 1..3: note = 2-aminoethoxy-2-ethoxy acetic acid
  VARIANT 4..5: note = diamino propionic acid
  DISULFID 12..28: note = intrachain disulfide bond
XXXXXSLRRSSCFGGRMDRIGAQSGLGCNSFRY

SEQ ID NO: 48 - protein, 32 aa, Homo sapiens
  REGION 1..32: note = native human B-type natriuretic peptide
SPKMVQGSGCFGRKMDRISSSSGLGCKVLRRH

SEQ ID NO: 49 - protein, 37 aa, synthetic construct
  VARIANT 1..3: note = 2-aminoethoxy-2-ethoxy acetic acid
  VARIANT 4..5: note = diamino propionic acid
  DISULFID 15..31: note = intrachain disulfide bond
XXXXXSPKQVQGSGCFGRKQDRISSSSGLGCKVLRRH

SEQ ID NO: 50 - protein, 37 aa, synthetic construct
  VARIANT 1..3: note = 2-aminoethoxy-2-ethoxy acetic acid
  VARIANT 4..5: note = diamino propionic acid
  DISULFID 15..31: note = intrachain disulfide bond
XXXXXSPKMVQGSGCFGRKMDRISSSSGLGCKVLRRH

SEQ ID NO: 51-69 - skipped (19 entries)

SEQ ID NO: 70 - protein, 7 aa, synthetic construct
KKGGGKK

SEQ ID NO: 71 - protein, 7 aa, synthetic construct
GGGKKKK

SEQ ID NO: 72 - protein, 7 aa, synthetic construct
KKKKGGG

SEQ ID NO: 73 - protein, 7 aa, synthetic construct
KKGGGRR

SEQ ID NO: 74 - protein, 7 aa, synthetic construct
  VARIANT 6..7: note = diamino butanoic acid
KKGGGXX

SEQ ID NO: 75 - protein, 32 aa, Homo sapiens
  REGION 1..32: note = Urodilantin
TAPRSLRRSSCFGGRMDRIGAQSGLGCNSFRY

(19) United States
(12) Patent Application Publication
(10) Pub. No.: US 2024/0325544 A1
(43) Pub. Date: Oct. 3, 2024

(54) CATIONIC LIPID FOR COVALENT MODIFICATION OF PEPTIDES

(71) Applicant: PharmalN Corporation, Bothell, WA (US)

(72) Inventors: Gerardo M. Castillo, Bothell, WA (US); Yao Yao, Seattle, WA (US); Rebecca E. Guerra, Bothell, WA (US); Kali A. Striker, Snohomish, WA (US); Elijah M. Bolotin, Bothell, WA (US); Hirofumi Tachibana, Fukuoka City (JP); Takashi Nojiri, Osaka City (JP); Motofumi Kumazoe, Fukuoka City (JP)

(21) Appl. No.: 18/622,227

(22) Filed: Mar. 29, 2024

Related U.S. Application Data
(60) Provisional application No. 63/493,290, filed on Mar. 30, 2023.

(51) Int. Cl. A61K 47/54; A61K 45/06; A61P 11/00; A61P 35/04

(52) U.S. Cl. CPC A61K 47/542; A61K 45/06; A61P 11/00; A61P 35/04

(57) ABSTRACT

Provided herein are compounds (e.g., peptides) comprising a cationic alkyl moiety, compositions comprising the compounds, and methods of using the compounds for, e.g., treating a disease, disorder, or condition.

### RELATED APPLICATION(S)

This application claims the benefit of U.S. Provisional Application No. 63/493,290, filed on Mar. 30, 2023. The entire teachings of the above application are incorporated herein by reference.

### GOVERNMENT SUPPORT

This invention was made with government support under grant number HL156295 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 6165.1024-002_SL.xml; created Mar. 29, 2024, 67,927 Bytes in size.

### BACKGROUND

Alkyl chain modification of native peptides is one of several approaches used to enhance pharmacokinetic properties and/or increase in vivo half-life of otherwise biologically unstable peptides (see, e.g., Menacho-Melgar et al, J Control Release. 2019. Feb. 10; 295: 1-12). To date, most modified peptides have utilized fatty acid or alkyl chains without cationic moieties, as the inherent toxicity of cationic alkyl or lipid compounds has been well documented (see, e.g., Cui et al, The Royal Society of Chemistry. 2018. pp 473-479). Using cationic lipids to modify a peptide drug is known to increase its toxicity, often resulting in a narrow therapeutic window where the toxic dose closely approaches the desired dose for biological activity. As a result, this approach is generally considered undesirable. Moreover, peptide modification can cause a loss of peptide biological activity or decreased solubility.

Despite these disadvantages, the use of cationic alkyl or lipid modifications can improve in vivo half-life by potentially interacting or binding with cell membrane and proteins. However, this same property also leads to increased toxicity that has been attributed to a very tight binding to cell membrane components causing either hemolysis, caspase induced cell apoptosis, mitochondrial dysfunction due to decreased membrane potential, increase in reactive oxygen species (ROS) levels, cell arrest at S phase and/or other unknown mechanism leading to toxicity. Accordingly, the use of cationic alkyl or lipid moieties in peptide modifications has been limited because of potential toxicities that can reduce therapeutic index. The toxicity of cationic lipids is strongly connected to the presence of several amino headgroups that have been shown to kill 50% of cells in culture at μM concentrations and higher. As a result, cationic lipid applications have been limited to killing cells with abnormally high negatively charge membrane compared to normal mammalian cells, such as cancer (see, e.g., Cui et al, The Royal Society of Chemistry. 2018. pp 473-479) and bacterial cells (see, e.g., Maluch et al, Int. J. Mol. Sci. 2020, 21, 8944).

### SUMMARY

Provided herein are compounds comprising a cationic alkyl moiety of Formula (I):

J-(CH2)x(CO)-(A)y-(B)z- (I),
wherein:
   J is either HOOC or CH3;
   x is 10-16;
   A is independently selected from the group consisting of: 2-[2-(2-Aminoethoxy)ethoxy]acetic Acid (Aeea), gamma amino butyric acid (γAbu), gamma linked glutamate (γE), and alpha linked glutamate (E);
   y is 2-4;
   B is independently Diamino propionic acid (Dap) or Diamino butanoic acid (Dab); and
   z is 2-4;
wherein -(B)z- comprises no more than 2 Dab residues and wherein the Dap or Dab residues are linked through the alpha amino.

Also provided herein are conjugated peptides of Formula (II):

CH3(CH2)x(CO)-(A)y-(B)z-Peptide (II),
wherein:
   x is 10-16;
   A is independently selected from the group consisting of: 2-[2-(2-Aminoethoxy)ethoxy]acetic Acid (Aeea), gamma amino butyric acid (γAbu), gamma linked glutamate (γE), and alpha linked glutamate (E);
   y is 2-4;
   B is independently Diamino propionic acid (Dap) or Diamino butanoic acid (Dab); and
   z is 2-4;
wherein:
   (B)z- comprises no more than 2 Dab residues and wherein the Dap or Dab residues are linked through the alpha amino;
   CH3(CH2)x(CO)-(A)y-(B)z- is covalently linked to the N-terminus of Peptide or linked to one of the side chain amino groups of Peptide.

In some embodiments, the conjugated peptide has: biological activity that is equivalent or higher than the unmodified peptide at an equivalent bolus dose; an equivalent or higher blood level than the unconjugated peptide at the same time-point after a bolus administration at an equivalent dose; or a combination thereof.

Provided herein are compositions comprising a compound or conjugated peptide of the disclosure for use in the manufacture of a medical composition.

In addition, provided herein are compounds, conjugated peptides, and compositions of the disclosure for use in treating a disease or condition in a subject in need thereof.

Also provided herein are compositions comprising a compound or conjugated peptide of the disclosure, and one or more pharmaceutically acceptable carriers or excipients.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1B: PK/PD Profiles for single subcutaneous (SC) injection of native CNP (SEQ ID NO: 32), SEQ ID NO: 31, and SEQ ID NO: 29 at 1.0 mg/kg (0.45, 0.33, and 0.33 μmol/kg, respectively) in mice. Error bars represent standard error of the mean. FIG. 1A) Plasma CNP [mean (SEM); n=5] in CD-1 mice after subcutaneous administration of 1.0 mg/kg of native CNP (SEQ ID NO: 32), SEQ ID NO: 31, and SEQ ID NO: 29 (0.45, 0.33, and 0.33 μmol/kg, respectively) in buffer. Between all groups, baseline CNP level prior to administration is 4.5 (1.4) ng/mL [mean (SD); n=15]. Sequence IDs 31 and 29 not only increase baseline plasma CNP levels by over 5- and 10-fold, respectively, but also demonstrate a sustained elevation of CNP levels when compared to native CNP. Plasma CNP in CD-1 mice was measured by CNP ELISA kit from Phoenix Pharmaceuticals (EKE-012-03, Burlingame, CA).

FIG. 1B) Plasma cGMP in CD-1 mice after subcutaneous administration of 1.0 mg/kg of native CNP (SEQ ID NO: 32), SEQ ID NO: 31, and SEQ ID NO: 29. Between all groups, baseline plasma cGMP level is 13.9 (5.7) μmol/mL [mean (SD); n=15]. SEQ ID NO: 31 and 29 not only increase baseline plasma cGMP levels by over 10-fold, but also demonstrate a sustained elevation of cGMP levels when compared to native CNP. Plasma cGMP was measured by cGMP ELISA kit from Abcam (ab133052, Waltham, MA).

FIGS. 2A-2B: Single and repeat administration of cationic alkyl modified C-type Natriuretic Peptides increases probability of survival in LPS induced Sepsis and ALI animal models. FIG. 2A) Sepsis induction: Male C57BL/6J mice (n=10 per group; 3 groups) were injected IP with LPS (15 mg/kg) and treated with various test articles, including cationic alkyl modified CNPs (SEQ ID NO: 31 and 29; 0.3 mg/kg (0.1 μmol/kg) SC). The control group received LPS treatment without any test article. The test articles were administered immediately after LPS administration (marked with vertical dotted lines). Survival was monitored every 2 hours from 8-56 hours, after which the surviving mice were euthanized under isoflurane anesthesia. Statistical analysis was based on Gehan-Breslow-Wilcoxon test performed by using GraphPad Prism (n=10, 10, and 10; Control, SEQ ID NO: 31, and SEQ ID NO: 29). ** P<0.01, * P<0.05 vs Control group. FIG. 2B) ALI induction: Male C57BL/6J mice (n=6 per group; 3 groups) were injected IT with LPS (20 mg/kg) and treated with various test articles, including cationic alkyl modified CNPs (SEQ ID NOs: 31 and 29; 0.3 mg/kg (0.1 μmol/kg) IT). The control group received LPS treatment without any test article. The test articles were administered immediately after LPS administration and repeated every 24 hours for a total of 3 bolus doses (marked with vertical dotted lines). Survival was monitored every 8 hours for 72 hours, after which the surviving mice were euthanized under isoflurane anesthesia. Statistical analysis was based on Gehan-Breslow-Wilcoxon test performed by using GraphPad Prism (n=6, 6, and 6; Control, SEQ ID NO: 31, and SEQ ID NO: 29). ** P<0.01, * P<0.05 vs Control group.

FIGS. 3A-3D: Bolus administration of cationic alkyl modified C-type Natriuretic Peptides suppress lung injury indicating resolution of ALI/ARDS. Increased neutrophil count and expression of myeloid cell-derived proteins (S100A8/A9) are elevated in several types of inflammatory lung disorders. MPO+ cells serve as a direct measure of neutrophil presence. Therefore, in the animal models of ALI (FIG. 3A), the expression of S100A8 and S100A9 (FIG. 3B) and presence MPO+ cells in the lungs (FIG. 3C, D) are measured as common markers of inflammation. The decreases indicate resolution of ALI/ARDS consistent with the observed increase survival (see FIG. 2). FIG. 3A) shows diagram of the procedure where C57BL/6J mice were administered LPS (0.05 mg/kg IT) followed by treatment with various test articles, including Sivelestat (150 mg/kg), an inhibitor of human neutrophil elastase, injected IP as a positive control, and cationic alkyl modified CNPs (SEQ ID NO: 31, 29, and 30; 0.3 mg/kg (0.1 μmol/kg) SC). The test articles were administered immediately after LPS injection. Additionally, a normal control (NC) group without LPS administration and a Control group receiving only LPS without any test article were included. After 24 hours, the mice were euthanized under isoflurane anesthesia and their lungs were harvested for analysis. Due to the different lung processing techniques between studies, it was necessary to repeat this procedure. FIG. 3B) Lungs were minced in Tri-Reagent and processed to measure the gene expression levels of S100A8 and S100A9 with qRT-PCR-analysis by using a cDNA synthesis kit (Qiagen; Venlo, the Netherlands). Statistical analysis was based on Dunnett's test performed by using GraphPad (n=5, 8, 8, 8, 8, 8, and 8; NC, Control, PC, A [SEQ ID NO: 31], B [SEQ ID NO: 29], Control, D [SEQ ID NO: 30]). *** P<0.001, ** P<0.01, * P<0.05 vs each corresponding Control group. FIG. 3C, D) Lung tissue was fixed, embedded in paraffin, and sectioned. Immunohistochemical staining was performed on the sections and the number of myeloperoxidase positive (MPO+) cells was quantified per field of view. Statistical analysis was based on Dunnett's test performed by using GraphPad (n=5, 8, 8, 8, 8, 8, and 8; NC, Control, PC, A [SEQ ID NO: 31], B [SEQ ID NO: 29]; Control, D [SEQ ID NO: 30]). *** P<0.001 vs each corresponding Control group.

FIGS. 4A-4C: Cationic alkyl modified CNP derivatives decreased neutrophil infiltration in the lung. ALI and ARDS are associated with increased cells in BALF, particularly neutrophils. To assess the resolution of ALI/ARDS in animal models, we measured the number of cells (FIG. 4B) and total protein levels (FIG. 4C), which serve as a marker for neutrophils. A decrease in these markers indicates the resolution of ALI/ARDS. FIG. 4A) shows a diagram of the procedure where mice were administered LPS (0.05 mg/kg IT) followed by treatment with various test articles, including Sivelestat (150 mg/kg), an inhibitor of human neutrophil elastase, injected IP as a positive control, and cationic alkyl modified CNPs (SEQ ID NO: 31, 29, and 30; 0.3 mg/kg (0.1 μmol/kg) SC). The test articles were administered immediately after LPS injection. Additionally, a normal control (NC) group without LPS administration and a Control group receiving only LPS without any test article were included. After 24 hours, the mice were euthanized under isoflurane anesthesia and BALF was harvested. Statistical analysis was based on Dunnett's test performed by using GraphPad (n=5, 8, 8, 8, 8, 8, and 8; NC, Control, PC, A [SEQ ID NO: 31], B [SEQ ID NO: 29]; Control, D [SEQ ID NO: 30]). *** P<0.001, vs each corresponding Control group.

FIGS. 5A-5E: The effect of cationic alkyl modified CNP derivatives in inflammation status of acute exacerbation of idiopathic pulmonary fibrosis (IPF-AE). Error bars indicate standard error of the mean. Increased inflammatory status in lung, especially increase of neutrophils, MCP1, IL-6, are seen in IPF-AE. Its decrease indicates resolution of IPF-AE. FIG. 5A) shows a diagram of the procedure where C57BL/6J mice were treated with bleomycin (Bleo; 1.0 mg/kg IT administration). After 3 weeks, mice were treated with LPS (0.025 mg/kg IT) and treated SC with SEQ ID NO: 29 or 31 at 0.3 mg/kg (0.1 μmol/kg) the day before, of, and after LPS treatment as shown in the diagram. Additionally, a normal control (NC) group without LPS/Bleo treatment, a Bleo group without LPS treatment, and a Control group without test article treatment were also included. On the final day, mice were sacrificed under isoflurane anesthesia and then lung tissues were harvested and weighted. FIG. 5B) Lung weight/body weight ratio increase is the parameter for lung damage. FIG. 5C) Upregulation of MPO, a marker for neutrophils, was significantly attenuated by groups treated with cationic alkyl modified CNP derivatives. FIG. 5D) SEQ ID NO: 31 in particular showed a significant decrease of MCP1, another common indicator of inflammation. FIG. 5E) IL-6 levels showed no significance compared to NC group, which indicates reduced inflammation compared to Control group. Statistical analysis was based on Student's t test performed by using GraphPad (n=5, 5, 8, 8, 8; NC, Bleo, Control, B [SEQ ID NO:29], A [SEQ ID NO:31]). ##P<0.01 and #P<0.05 vs Control.

FIGS. 6A-6B: Repeat subcutaneous administration of cationic alkyl modified CNP derivatives demonstrated significant anti-tumor activity in an orthotopic mouse model of breast cancer using E0771 cells. Error bars represent standard error of the mean. FIG. 6A) Tumor growth kinetics of 6-week-old female C57BL/6J mice (n=10 per group) that were inoculated with E0771 breast cancer cells (250,000 cells/mouse) in the mammary gland. Starting from Day 4 post-inoculation, cationic alkyl modified CNP derivatives (SEQ ID NO: 29, 30, and 31) were administered subcutaneously at a bolus dose of 0.3 mg/kg (0.1 μmol/kg) once daily for 5 days (5 days on, 2 days off) for 3 cycles; dose days indicated by gridlines. The control group, which received only buffer, was dosed in the same manner to establish the baseline tumor growth kinetics. FIG. 6B) The groups treated with cationic alkyl modified CNP showed a significant decrease in tumor volume compared to the Control group at the conclusion of the study. However, cationic alkyl sequence without CNP (SEQ ID NO: 14) was also tested in this model but did not show a significant reduction in tumor volume. Therefore, it can be concluded that the attachment of CNP is important for the observed anti-tumor activity. Statistical analysis was based on Dunnett's test performed by using GraphPad (n=10, 10, 10, and 10; Control, SEQ ID NO: 29, SEQ ID NO: 30, and SEQ ID NO: 31) **** P<0.0001 vs Control group.

FIG. 7: Binding of CNP derivative to both natriuretic peptide receptor B (NPRB) and natriuretic peptide receptor C (NPRC). Error bars represent standard error of the mean. The CNP based probe alone (5 nM CNP-F*) has rapid rotation and results in low fluorescence polarization (FP) signal. When human NPRB or NPRC (50 nM) was added, the CNP probe binds to those receptors and results in slow rotation and high FP signal. In the presence of NPRA, no FP signal change was detected, which suggests CNP probe does not bind to NPRA. In the presence of SEQ ID. NO. 31, the low FP signal demonstrates binding to both NPRB and NPRC.

FIGS. 8A-8B. The use of cationic alkyl modified CNP derivative alone or with pirfenidone demonstrated a reduction of fibrosis in mouse model of idiopathic pulmonary fibrosis (IPF). FIG. 8A) Shows a diagram of the procedure to induce pulmonary fibrosis where male C57BL/6J mice (6 weeks-old) received IT administration of bleomycin (Bleo; 1.0 mg/kg) and after 7 days, SEQ ID NO: 31 was administered SC once a day at 0.3 mg/kg on weekdays and/or pirfenidone once daily (100 mg/kg orally). Additionally, a normal control group (NC; n=3) without Bleo, and a Bleo Control group (n=7) without test article treatment were also included in this study. On Day 21 post Bleo administration, the mice were sacrificed under isoflurane anesthesia, and their lung tissues were harvested. FIG. 8B) A part of the lung tissue was fixed in 4% PFA, and Azan staining was performed, and alveolar areas (inversely proportional to the degree of fibrosis) were measured by Image J in a blinded setting. Error bars indicate standard error of the mean. Statistical analysis was performed by Student t-test on GraphPad Prism (n=3, 7, 7, 7, and 7); NC, Bleo Control, SEQ ID NO: 31, Pir, and Combo (SEQ ID NO: 31 & pirfenidone) *** P<0.001, ns=not significant vs NC. Groups treated with SEQ ID NO: 31 alone or in combination with pirfenidone showed a significantly higher alveolar area, which demonstrates the presence of healthy tissue and a reduction in fibrosis.

FIG. 9. Repeat subcutaneous administration of cationic alkyl modified CNP derivative as a monotherapy or in combination with an immune check point inhibitor significantly reduced tumor volume in an orthotopic mouse model of breast cancer using E0771 cells. Error bars represent standard error of the mean. Tumor growth kinetics of 6-week-old female C57BL/6J mice (n=7-8 per group) that were orthotopically inoculated with E0771 breast cancer cells (250,000 cells/mouse) in the mammary gland. Starting from Day 4 post-inoculation, anti-PD1 Ab (aPD1) was administered intraperitoneally at 5 mg/kg twice a week for 2 cycles; SEQ ID NO: 31 was administered subcutaneously at a bolus dose of 0.3 mg/kg once daily for 5 days (5 days on, 2 days off) for 3 cycles; or a combination of aPD1 (5 mg/kg IP twice a week; 2 cycles) and SEQ ID NO: 31 (0.3 mg/kg SC once a day for 5 days on and 2 days off; 3 cycles); SEQ ID NO: 31 dose days indicated by dotted line or gridlines. The control group, which received only buffer, was dosed in the same manner as SEQ ID NO: 31 to establish the baseline tumor growth kinetics. At the conclusion of the study the groups treated with SEQ ID NO: 31 showed antiproliferative effects or a significant decrease in tumor volume compared to the Control group and the group treated solely with aPD1, an immune check point inhibitor. Statistical analysis was based on Dunnett's test performed by using GraphPad (n=8, 7, 8, and 7; Control, aPD1, SEQ ID NO: 31, Combo (aPD1 and SEQ ID NO: 31) * P<0.05, **** P<0.0001 vs Control group.

FIGS. 10A-10B. The combination of radiation, an immune checkpoint inhibitor, and repeat subcutaneous administration of cationic alkyl modified CNP derivative reduced incidence of metastasis and significantly improved overall survival in orthotopic mouse model of bone metastasis using breast cancer E0771 cells. Error bars indicate standard error of the mean. FIG. 10A) Shows a diagram of the procedure where female C57BL/6J mice (6 weeks-old) were orthotopically implanted with E0771 breast cancer cells (250,000 cells/mouse in RPMI1640 medium) in their left mammary gland and E0771 mouse breast cancer cells (500,000 cells/mouse in 50% Matrigel) in the femur. Also illustrated in this diagram are the corresponding schedules for each treatment administered throughout this study. From tumor implantation, SEQ ID NO: 31 treated groups (2, 3, 6, and 7) were dosed SC (0.3 mg/kg/0.1 μmol/kg in buffer 15 mM succinate, 4% (w/v) D-mannitol, 10 mM hydoxypropyl-beta-cyclodextrin pH 4.4) on Days 5-9, 12-16, 19-23, and 26-29; aPD1 treated groups (3, 5, and 7) were dosed IP (5 mg/kg) on Days 5, 7, 12, and 14; and irradiated groups (4, 5, 6, and 7) received buffer and 5 Gy of X-ray radiation in three sets at the bone on Days 5, 8, and 12. Survival was observed until Day 33, following scheduled sacrifice of remaining mice. At this point, tumor sizes were measured using calipers. FIG. 10B) Shows the probability of survival for groups 1 to 7. All groups were significant compared to the control group, so statistical analysis was based on Log-rank (Mantel-Cox) test against group 5 (radiation+aPD1) using GraphPad Prism. With the addition of SEQ ID NO: 31, the combination of all 3 treatments demonstrated a significant improvement in survival (** P<0.01). Furthermore, 5 out of 6 mice in group 6 (SEQ ID NO: 31+radiation) and 6 out of 6 mice in group 7 (SEQ ID NO: 31+radiation+aPD1) were tumor free in the bone compared to 1 out of 5 mice in groups 4 (radiation alone) and 5 (radiation+aPD1), which demonstrates reduced incidence of metastasis and cancer burden with the addition of SEQ ID NO: 31.

FIGS. 11A-11C. Amputation in combination with repeat subcutaneous administration of cationic alkyl modified CNP derivative significantly reduced incidence of lung metastasis in orthotopic mouse model of lung metastasis using osteosarcoma LM8 cells compared to amputation alone. Error bars indicate standard error of the mean. FIG. 11A) Shows a diagram of the procedure where male CH3/He mice (7 weeks-old) were orthotopically implanted with LM8 osteosarcoma cells in their femur (1,000,000 cells/mouse). Also illustrated in this diagram are the corresponding treatment schedules for SEQ ID NO: 31 and amputation. From tumor implantation, the SEQ ID NO: 31 treated group was dosed SC (0.3 mg/kg/0.1 μmol/kg in buffer) on Days 4-8, 11-15, 18-22, and 25-26. On Day 7 post-inoculation, all mice underwent amputation to remove the primary tumor. In place of treatment, the amputated control group also received buffer on the same days that SEQ ID NO: 31 was administered. Mice were sacrificed on Day 34, and lung tissues were harvested. FIGS. 11B and 11C) Lung tissues were soaked in 4% paraformaldehyde, paraffin-embedded, and sections were stained with hematoxylin and eosin (H&E). Lung images were observed using high-resolution microscopy (Keyence, Tokyo, Japan, #BZ-X700), and lung metastasis was evaluated by the direct count of present metastatic lung nodules. The SEQ ID NO: 31 treated group with amputation had a significant reduction (** P<0.01) of metastatic nodules present in the lung when compared to the group that underwent amputation with no added treatment. Outliers were identified using the ROUT test (Q=1%). All statistical analysis was based on Dunnett's test performed by using GraphPad (n=7 and 7; Control (amputation), Combo (amputation and SEQ ID NO: 31)) ** P<0.01 vs Control group.

FIGS. 12A-12B. Repeat subcutaneous administration of cationic alkyl modified CNP derivative as a monotherapy or in combination with an immune check point inhibitor significantly reduced tumor volume in a subcutaneous mouse model of colon cancer using MC38 cells. Error bars represent standard error of the mean. FIG. 12A) A diagram of the dose schedule for administration of immune checkpoint inhibitor (anti-tigit Ab) and SEQ ID NO: 31. FIG. 12 B) Tumor growth kinetics of 6-week-old male C57BL/6J mice (n=8-9 per group) that were subcutaneously inoculated with MC38 colon cancer cells (1,000,000 cells/mouse) in right flank. Starting from Day 4 post-inoculation, anti-tigit Ab was administered intraperitoneally at 5 mg/kg twice a week for 2 cycles; SEQ ID NO: 31 was administered subcutaneously at a bolus dose of 0.3 mg/kg (0.1 μmol/kg) once daily for 5 days (5 days on, 2 days off) for 3 cycles; or a combination of anti-tigit Ab (5 mg/kg IP twice a week; 2 cycles) and SEQ ID NO: 31 (0.3 mg/kg SC once a day for 5 days on and 2 days off; 3 cycles). The control group, which received only buffer, was dosed in the same manner as SEQ ID NO: 31 to establish the baseline tumor growth kinetics. The groups treated with SEQ ID NO: 31 showed a significant decrease in tumor volume and cancer burden compared to the Control group and the combination group (anti-tigit Ab+SEQ ID NO: 3) outperformed the anti-tumor effect of anti-tigit Ab as a monotherapy. Statistical analysis was based on Dunnett's test performed by using GraphPad (n=9, 8, 8, and 8; Control, anti-tigit Ab, SEQ ID NO: 31, Combo (anti-tigit Ab and SEQ ID NO: 31) * P<0.05 vs anti-tigit Ab monotherapy group.

FIG. 13: HeLa cells treated with cationic alkyl modified CNP derivative demonstrated binding to NPR—C based on significant inhibition of baseline cyclic adenosine monophosphate levels. In this study, HeLa cells were cultured in Dulbecco modified Eagle medium (DMEM) supplemented with 10% FBS under 100% humidity and 5% CO2 at 37° C. The cells were harvested and suspended at a concentration of 10⁷ cells/mL in ENGS and 5 uL of the cell suspension was added to each well of a 96-well low-adhesion plate. The cells (n=4 wells) were then treated with 5 uL of SEQ ID NO: 31 (final concentration of 10 μg/mL) in ENGS with 1 mM IBMX for 10 minutes. cAMP levels were evaluated using a cAMP assay kit (PerkinElmer, Waltham, MA, USA, #62AM4PEB) in accordance with its manufacture's protocol with a PerkinElmer plate reader. Statistical analysis was performed with GraphPad Prism against untreated control wells (n=4 wells) * P<0.05. The NPR-C receptor has been implicated in inhibiting adenylate cyclase activity, which would lead to a decrease in cyclic adenosine monophosphate (cAMP) production. The conclusion of this study resulted in changes (inhibition) of cAMP baseline levels of HeLa cells (known to express NPR-C) treated with SEQ ID NO: 31, which indicates binding to NPR-C.

### DETAILED DESCRIPTION

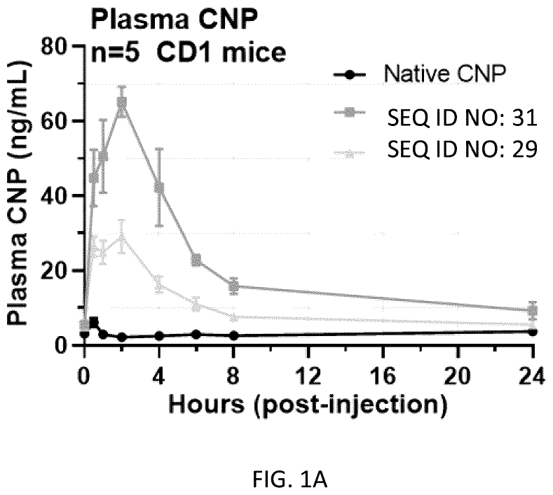

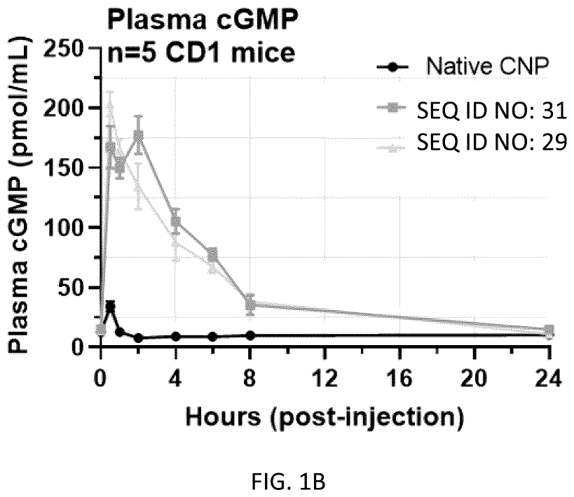

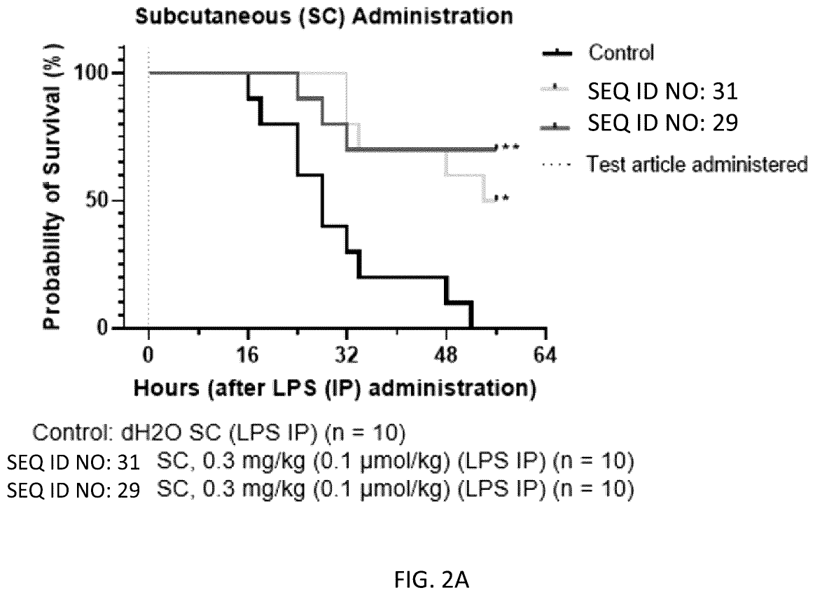

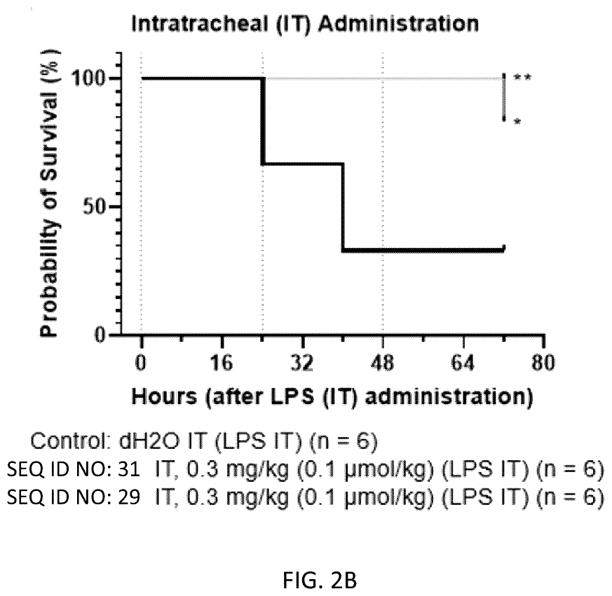

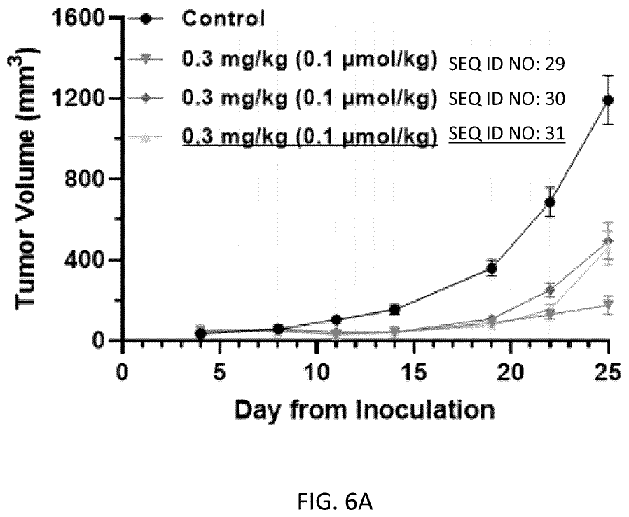

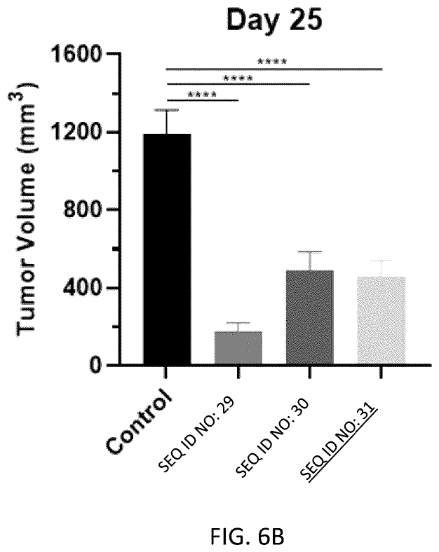

The present disclosure provides novel compositions comprising compounds that comprise a single cationic alkyl chain moiety, which is useful for modifying biologically active molecules, such as peptides, and which exhibits surprising and significantly reduced toxicity when administered in vivo (for example, no toxicity or ataxia was observed for particular modifiers disclosed herein at 10 μmol/kg in rat). The novel compositions comprise compounds comprising a single alkyl chain linked by a non-cationic linker to a chain of 2-4 cationic amino acid residues selected from diamino propionic acid (Dap) and diamino butanoic acid (Dab), wherein there are no more than 2 Dab residues in the chain. The non-cationic linker comprises 2-4 residues independently selected from 2-aminoethoxy-2-ethoxy acetic acid (Aeea), gamma amino butyric acid (γAbu), gamma linked glutamate (γE), and glutamate (E). The cationic alkyl chains disclosed herein can be linked to a peptide to enhance the peptide's pharmacokinetic and/or pharmacodynamic activity (see, e.g., FIG. 1), for example, relative to the native unmodified peptide and without the risk of causing additional toxicity (e.g., at a bolus dose of 3.0 μmol/kg or less, Table 1). As exemplified in the present specification, the cationic alkyl chain modified peptide, such as C-type natriuretic peptide (CNP) (or a derivative thereof), can be used to increase survival from sepsis and/or acute respiratory distress syndrome/acute lung injury and/or pulmonary fibrosis (See FIGS. 2-5 and 8 and Examples 5-9 and 12 herein). Additionally, cationic alkyl chain modified CNP (or a derivative thereof) can be used as a therapeutic agent, for example, to treat cancer (see FIGS. 6 and 9-12 and Examples 10 and 13-16), and without reaching dose causing toxicity/ataxia. Additionally, exemplified in the present specification are cationic alkyl chain modified atrial natriuretic peptide, B-type natriuretic peptide, and their derivatives.

### Acute Lung Injury and Acute Respiratory Distress Syndrome

Acute lung injury (ALI) and acute respiratory distress syndrome (ARDS) are clinical conditions that feature an acute onset of arterial hypoxemia, with ALI characterized by a PaO2/FiO2 ratio of less than 300 Torr and less than or equal to 200 Torr for the more severe ARDS, along with bilateral radiographic infiltrates and no evidence of left atrial hypertension (see, e.g., Bernard, G. R., et al., J. Crit. Care, 1994. 9(1): p. 72-81; Rubenfeld, G. D., et al., N Engl J Med, 2005. 353(16): p. 1685-93; Brun-Buisson, C., et al., Intensive Care Med, 2004. 30(1): p. 51-61; and Phua, J., et al., Am J Respir Crit Care Med, 2009. 179(3): p. 220-7). As used herein, PaO2 refers to the partial pressure of arterial oxygen, and FiO2 is the fraction of oxygen in the inspired air (for reference, room air has a FiO2 of about 0.21, and normal PaO2/FiO2 is about 500 Torr). Both ALI and ARDS result in death or pulmonary fibrosis. ARDS is an overwhelming pulmonary inflammatory response to certain primary and secondary noxious stimuli such as pneumonia (e.g., aseptic pneumonia, viral pneumonia, bacterial pneumonia), sepsis, aspiration, inhalation injuries, near drowning, and pulmonary resection surgery (see, e.g., Alam, N., et al., Ann Thorac Surg, 2007. 84(4): p. 1085-91). ARDS is characterized by rapid-onset respiratory failure necessitating hospitalization and ventilatory support in an intensive care unit (ICU). If a patient survives ALI/ARDS, the long-term quality of life of the patient is often adversely affected due to lung scarring (see, e.g., Rubenfeld, G. D., et al., N Engl J Med, 2005. 353(16): p. 1685-93; Dowdy, D. W., et al., Intensive Care Med, 2006. 32(8): p. 1115-24). To date, no effective agent to treat acute lung injury (ALI) and ARDS has been identified and there is a significant need for such an agent. Agents previously tested in human clinical trials for the treatment of ALI, including glucocorticoids, surfactants, N-acetylcysteine, inhaled nitric oxide, liposomal PGE 1, ketoconazole, lisofylline, salbutamol, procysteine, activated protein C, and inhaled albuterol, have all failed (see, e.g., Johnson E R and Matthay M A, J Aerosol Med Pulm Drug Deliv. 2010, 23(4):243-52). Medicament for the treatment of ALI or ARDS in human remains elusive to the person skilled in the art. Supportive care for ALI includes oxygen to maintain arterial partial pressure of oxygen (PaO2) above 55 mmHg, or oxygen saturation (SaO2) above 88%, and fluid management. However, care must be taken not to provide too much oxygen (i.e., oxygen should be given at less than 60%) to avoid oxygen toxicity. Moreover, this measure does not address the underlying alveolar inflammatory edema (fluid in the lung alveoli filled with blood protein and inflammatory cells). The underlying alveolar inflammatory edema in ALI or ARDS is the root cause of low blood oxygenation.

### Lung or Pulmonary Fibrosis

Lung or Pulmonary Fibrosis (PF) is a progressive scarring of the lung tissue caused by many conditions including infections (i.e., sepsis or pneumonia that can be aseptic, viral or bacterial in origin), environmental agents (e.g., asbestos, silica, exposure to certain gases), exposure to ionizing radiation (e.g., radiation therapy to treat tumors of the chest), chronic auto immune conditions (e.g., lupus, rheumatoid arthritis), chronic inflammatory processes (e.g., sarcoidosis, Wegener's granulomatosis), or certain medications. Interstitial lung disease (ILD) is another umbrella term used for PF and for the purpose of this specification will be synonymous. Idiopathic pulmonary fibrosis (IPF) is a PF of unknown cause. PF or IPF are an incurable type of chronic scarring lung disease characterized by a progressive and irreversible decline in lung function with gradual onset of shortness of breath and a dry cough that affects 5 million people globally (see, e.g., Raghu G, Collard H R, Egan J J, et al., (2011) American Journal of Respiratory and Critical Care Medicine. 183 (6): 788-824) with associated risk factors that include chemical inhalation such as cigarette smoking, viral infections, or a family history of the condition. Other symptoms may include fatigue, and abnormally large and dome-shaped fingernails and toenails (nail clubbing). See, e.g., the National Institute of Health's health topics page on idiopathic pulmonary fibrosis and the Wikipedia page for idiopathic pulmonary fibrosis. Complications may include pulmonary hypertension, heart failure, pneumonia, or pulmonary embolism.

Thus, there is a need for safer and more efficacious composition for the treatments of ALI and/or ARDS, and PF which avoid the cardiovascular side-effects, such as hypotension, while maintaining or enhancing plasma levels of a CNP therapeutic agent. There is a need for a natriuretic peptide derivative with long half-life for use in the manufacture of a medicament for the treatment of ALI and/or ARDS. The present disclosure seeks to fulfill these needs and provides further related advantages.

### Natriuretic Peptide (NP) and Natriuretic Peptide Receptor (NPR)

Natriuretic peptide (NP) is a peptide that induces excretion of sodium in the kidney and lowers blood pressure though vasodilation by binding to the natriuretic transmembrane receptors with intracellular guanylate cyclase domain. Upon binding, the guanylate cyclase activity is activated, resulting in increased blood and intracellular cGMP levels and expression of various physiological activities. There are several natriuretic peptides well known in the arts. The C-type natriuretic peptide (CNP; GLSKGCFGLKLDRIGSMSGLGC [SEQ ID NO: 32]) acts through Natriuretic Peptide Receptor B (NPRB) and Natriuretic Peptide Receptor C (NPRC) (see, e.g., Silver M A, Curr. Opin. Nephrol. Hypertens., 2006, vol. 15,14-21; Yoshibayashi M. et al., Eur. J. Endocrinol., 1996, vol. 135, 265-268; Itoh H and Nakao K, Nihon Rinsho, 1997; 55: 1923-1936; Koller J K, et al., Science. 1991; 252: 120-123; Suga S, et al., Endocrinology. 1992; 130: 229-239; and Potter L R and Hunter T. J. Biol. Chem. 2001; 276:6057-6060.) whereas Atrial Natriuretic Peptide (ANP;SLRRSSCFGGRMDRIGAQSGLGCNSFRY; [SEQ ID NO: 44]), Urodilantin (URO; TAPRSLRRSSCFGGRMDRIGAQSGLGCNSFRY [SEQ ID NO: 75]), a longer version of ANP, and Brain Natriuretic Peptide (BNP; SPKMVQGSGCFGRKMDRISSSSGLGCKVLRRH; [SEQ. IN. NO. 48]) bind or act through Natriuretic Peptide Receptor A (NPRA). CNP, BNP, ANP, and URO are peptides having a cyclic structure necessary for their activity that is made possible by the presence of disulfide bonds. NPs have various physiological activities besides vasodilating action, blood-pressure, and vascular fluid regulation through diuretic action. For example, the inhibitory actions of ANP on bacterial infection-induced inflammation and associated failure in the endothelial barrier function have been reported (see, e.g., Xing J., et al., J. appl. Physiol., 2011, 110 (1), 213-224). Further, CNP, BNP, ANP, and URO all bind to NPRC (which lacks guanylyl cyclase activity) and undergo clearance and degradation (see, e.g., Koller K J, et al., Science. 1991; 252:120-123; Suga S, et al., Endocrinology. 1992; 130:229-239; and Potter L R and Hunter T. J. Biol. Chem. 2001; 276:6057-6060).

Conventionally, natriuretic peptide must be given continuously at low dosages because it has a very short half-life and a high bolus dose results in a very high peak plasma concentration (Cₘₐₓ) and a dangerous drop in blood pressure resulting in lethargy and ataxia, the main cause of toxicity. To mitigate these deleterious effects, natriuretic peptide is usually delivered by slow infusion. See, e.g., Kimura et al., J Surg Res. 2015, 194(2); 631-637. One aspect of the disclosed invention mitigates the natriuretic peptide toxicity from bolus administration while increasing cyclic-GMP response and efficacy in the treatment of diseases.

The present disclosure relates to the surprising and unexpected discovery of compounds comprising cationic alkyl moieties with low toxicity (e.g., a high dose is needed before ataxia is observed). The cationic alky moieties of the present disclosure can be covalently linked to molecules (e.g., peptides) to decrease in vivo degradation and/or prolong the presence in the blood or half-life without causing toxicity (ataxia). These benefits can occur at doses equal to or surpassing the therapeutically effective dose and at higher doses than the same peptide modified using other cationic alkyl moieties (see Table 3 in Example 3). Additionally, the disclosed composition can enhance biological activity (pharmacodynamics) compared to the unmodified peptide.

The present disclosure relates to compositions, including compounds, comprising a single alkyl chain with at least 2 positive charges derived from diamino propionic acid (Dap) and/or diamino butanoic acid (Dab) (defined here as cationic alkyl) with a surprising and non-obvious characteristic of significantly reduced toxicity (as tested by ataxia in rats) compared to those modified using cationic amino acids having larger, or longer chain, R-groups, including natural or unnatural D-form amino acids, such as lysine or arginine. The novel compositions comprise a single alkyl chain linked by non-cationic linker to 2-4 cationic amino acid residues independently selected from diamino propionic acid (Dap) and/or diamino butanoic acid (Dab), and wherein the cationic chain has no more than 2 Dab residues. The non-cationic linker is 2-4 residues independently selected from 2-aminoethoxy-2-ethoxy acetic acid (Aeea), gamma amino butyric acid (γAbu), gamma linked glutamate (γE), and glutamate €. The cationic alkyl composition of the present disclosure can be linked to a peptide to enhance its pharmacokinetic properties, bioavailability, and/or pharmacodynamic activity (see Example 4). As exemplified in the present specification, the cationic alkyl modified peptide, such as C-type natriuretic peptide (CNP) (or its derivative), can be used to increase survival from sepsis, acute respiratory distress syndrome, acute lung injury, and/or pulmonary fibrosis (see Examples 5-9 and 12 with associated Figures or Tables). Additionally, cationic alkyl modified CNP (or its derivative) can be used to treat or suppress cancer (see Examples 10 and 13-16). The present disclosure relates to a surprising discovery of safer cationic alkyl compositions that are safe at a bolus dose of up to 10 μmol/kg and lower (see Table 1 in Example 1). Additionally, the cationic alkyl moiety of the present disclosure can be covalently linked to therapeutic peptides in general for the purpose of increasing the peptide's in vivo stability, half-life (pharmacokinetics) and/or activity (pharmacodynamics) without causing toxicity or ataxia at a bolus dose that is at least twice the therapeutically effective dose and/or significant (measurable) pharmacodynamic activity. In other words, peptide modification using a cationic alkyl modification of the present disclosure does not cause toxicity to the peptide at a dose that provides significant pharmacodynamic and/or therapeutic activity compared to unmodified peptide. The present disclosure also relates to cationic alkyl-linked natriuretic peptides and their derivatives or compositions thereof, having unexpectedly superior ability to increase blood cGMP compared to native natriuretic peptide and with low toxicity compared to other cationic alkyl linked natriuretic peptides. Exemplification in the present disclosure of cationic alkyl linked peptides includes C-type natriuretic peptide (CNP), atrial natriuretic peptide (ANP), brain type natriuretic peptide (BNP), and their corresponding derivatives with an unexpectedly superior biological activity and/or lower toxicity compared to their corresponding native peptide or other modified natriuretic peptides known in the art.

For peptides to be considered for parenteral drug development, therapeutic efficacy must be present without toxicity when administered parenterally (e.g., by subcutaneous, intramuscular, inhalation, or intravenous administration) at practical or reasonable mass dose. For subcutaneous, intramuscular, and inhalation administrations of a peptide; the preferable and practical mass bolus dose in human should not exceed 12 mg/human, so as not to encounter a large volume injectate and/or peptide concentration beyond peptide solubility in the injectate. The human bolus dose of 12 mg/70 kg (or 0.17 mg/kg) will translate, after allometric scaling, to rat and mouse doses of 1.0 mg/kg and 2.0 mg/kg, respectively. Therefore, for a 5 kDa peptide, the mole dose in rat and mouse will be 0.20 μmol/kg and 0.40 μmol/kg, respectively. For a 1 kDa peptide, the mole dose in rat and mice will be 1.0 μmol/kg and 2.0 μmol/kg, respectively. Therefore, to ensure that the cationic lipid or cationic alkyl moiety that will be linked 1:1 (mole:mole) with the peptide will not add toxicity/adverse effect at a practical or reasonable therapeutic dose, the MTD or no observed adverse effect dose of the cationic alkyl moiety must be at least 2 times greater than the preferable and/or practical therapeutic mole dose of the peptide in the same species.

For the purpose of the present disclosure, the preferred compositions of the current invention comprise compounds comprising cationic alkyl chains of the disclosure, which, in some embodiments, have the ability to improve in vivo pharmacokinetics, pharmacodynamics, and/or bioavailability of the modified compound (e.g., peptide) with minimal or no observed toxicity (ataxia) in rat at a bolus dose of up to 10 μmol/kg (see Table 1 in Example 1). For cationic alkyl moieties that can improve the in vivo pharmacokinetics and/or pharmacodynamics of the peptide, the cationic alkyl moieties that do not cause toxicity (or ataxia) when administered by itself at bolus dose of at least 10 μmol/kg, 9.0 μmol/kg, 8.0 μmol/kg, 7.0 μmol/kg, 6.0 μmol/kg, or 5.0 μmol/kg (see Table 1 in Example 1) are preferred. This preference will ensure that the cationic lipid or cationic alkyl moiety that will be linked 1:1 on a molar basis with a peptide to prolong half-life and/or increase potency will not add toxicity/adverse effect to the peptide at doses of 3.0 μmol/kg and lower. The present disclosure discloses compositions comprising alkyl cationic moieties that can be attached or covalently linked in a 1:1 ratio with a peptide without the potential to cause additional toxicity to the peptide when administered at the preferable and practical bolus dose of 3.0 μmol/kg in rat or lower. As an example, covalently linking to natriuretic peptides a cationic alkyl moiety of the present disclosure that by itself does not cause ataxia at 10 μmol/kg or less in rat resulted in a conjugate that has unexpectedly superior ability to increase blood cGMP and/or intracellular cGMP in vivo compared to the native peptide, such as atrial natriuretic peptide (ANP) or brain type natriuretic peptide (BNP) or C-type natriuretic peptide (CNP) and their derivatives (see Table 3 in Example 3).

The present disclosure describes the modification of peptides or their derivatives in order to increase their half-life without added risk of toxicity at therapeutic doses as a result of the cationic alkyl modification. The modifiers of the present disclosure are cationic lipids which are known in the art to be generally toxic. However, when 2 or more Diamino propionic acid (Dap) or Diamino butyric acid (Dab) unnatural amino acids formed the cationic portion of the cationic alkyl modifiers and these cationic amino acids were separated from the alkyl chain with a noncationic spacer such as 2-4 residues of Aeea, γAbu, γE, and/or E, they were surprisingly found to have low toxicity (i.e., no ataxia and/or death) in rat. This is in contrast to alkyl modifications with cationic amino acids with larger R-groups (natural or unnatural D-form) such as lysine and without a noncationic spacer between the alkyl chain and cationic amino acids. This invention allows for the modification of peptides with cationic alkyl/lipid modifications to limit the added toxicity to the peptide. This provides a greater safety margin at or above the dose needed for biological activity (pharmacodynamics) or the practical and reasonable bolus dose (3.0 μmol/kg or lower for parenteral administration), which is critical in commercial drug development.

Most peptides that have been modified to date use alkyl chains without cationic moieties because cationic lipids or cationic alkyls have been well documented to be inherently toxic [Cui et al, 2018]. The present disclosure provides cationic alkyl compositions that surprisingly have very limited biological toxicity compared to other cationic alkyl compositions. Additionally, the cationic alkyl compositions of the present disclosure can be covalently linked to peptides at residues that are not essential to the peptide's biological activity, resulting in a peptide composition with an in vivo half-life or blood presence after bolus administration that is increased compared to the unmodified peptide. Additionally, because the cationic alkyl moiety on the modified peptide can interact with the anionic lipid membrane of the cells in combination with the specific interaction of the peptide with its receptor, the cationic alkyl moiety can increase the biological activity of peptide or the pharmacodynamic potency. The cationic alkyl compositions of the present disclosure can be attached or covalently linked to peptides, for example natriuretic peptides. The compositions and methods of use of the present disclosure are described herein.

A description of example embodiments follows.

### Compositions

### Formula (I)

The present disclosure provides a cationic alkyl or cationic lipid compound (e.g., for covalently modifying peptides, for example, to improve efficacy), wherein the compound comprises a cationic moiety of Formula (I):

J-(CH2)x(CO)-(A)y-(B)z; (I)
wherein:
J is either HOOC or CH3;
x is 10-16;
A is independently selected from the group consisting of: 2-[2-(2-Aminoethoxy)ethoxy]acetic Acid (Aeea), gamma amino butyric acid (γAbu), gamma linked glutamate (γE), and glutamate (E);
y is 2-4;
B is independently selected from Diamino propionic acid (Dap) or Diamino butanoic acid (Dab);
z- is 2-4;
where -(B)z- has no more than 2 Dab residues in the sequence and the linkages between residues are by amide bond to the alpha amino of Dab and/or Dap;
and wherein the composition produces no clinically observable toxic effect or ataxia after subcutaneous bolus administration in rats at a dose of 10 μmol/kg or lower.

In the determination of toxicity (where the observation of ataxia is used as a marker of toxicity), the doses were escalated until ataxia (i.e., toxicity) was seen, up to 10 μmol/kg, which is the maximum practical bolus dose of liquid parenteral peptides administered in a small volume of injectate. As used in the present disclosure, “ataxia” is a clinical sign observed as poor muscle control that causes clumsy voluntary movements. Ataxia may cause difficulty with mobility, coordination, and/or eye movements.

One embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), and gamma amino butyric acid (γAbu); and z is 2.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), and gamma amino butyric acid (γAbu); and z is 2.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), and gamma amino butyric acid (γAbu); y is 3; and z is 2.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), and gamma amino butyric acid (γAbu); y is 3; and z is 2.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), gamma linked glutamate (γE); and z is 2.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), gamma linked glutamate (γE); and z is 2.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), gamma linked glutamate (γE); y is 3; and z is 2.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), gamma linked glutamate (γE); y is 3; and z is 2.

Another embodiment of the present disclosure is a compound comprising a cationic moiety of Formula (I), wherein J is CH3, x is 10, 12, 14, or 16; A is 2-[2-(2-Aminoethoxy)ethoxy]acetic Acid (Aeea); and z is 2.

Another embodiment of the present disclosure is a compound comprising a cationic moiety of Formula (I), wherein J is CH3, x is 14; A is 2-[2-(2-Aminoethoxy)ethoxy]acetic Acid (Aeea); and z is 2.

Another embodiment of the present disclosure is a compound comprising a cationic moiety of Formula (I), wherein J is CH3, x is 10, 12, 14, or 16; A is 2-[2-(2-Aminoethoxy)ethoxy]acetic Acid (Aeea); y is 3; and z is 2.

Another embodiment of the present disclosure is a compound comprising a cationic moiety of Formula (I), wherein J is CH3, x is 14; A is 2-[2-(2-Aminoethoxy)ethoxy]acetic Acid (Aeea); y is 3; and z is 2.

Another embodiment of the present disclosure is a compound comprising a cationic moiety of Formula (I), wherein J is CH3, x is 10, 12, 14, or 16; A is gamma amino butyric acid (γAbu); and z is 2.

Another embodiment of the present disclosure is a compound comprising a cationic moiety of Formula (I), wherein J is CH3, x is 14; A is gamma amino butyric acid (γAbu); and z is 2.

Another embodiment of the present disclosure is a compound comprising a cationic moiety of Formula (I), wherein J is CH3, x is 10, 12, 14, or 16; A is gamma amino butyric acid (γAbu); y is 3; and z is 2.

Another embodiment of the present disclosure is a compound comprising a cationic moiety of Formula (I), wherein J is CH3, x is 14; A is gamma amino butyric acid (γAbu); y is 3; and z is 2.

One embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), and gamma amino butyric acid (γAbu); and z is 3.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), and gamma amino butyric acid (γAbu); and z is 3.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), and gamma amino butyric acid (γAbu); y is 3; and z is 3.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), and gamma amino butyric acid (γAbu); y is 3; and z is 3.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), gamma linked glutamate (γE); and z is 3.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), gamma linked glutamate (γE); and z is 3.

Another embodiment of the present disclosure is a compound comprising a cationic moiety of Formula (I), wherein J is CH3, x is 10, 12, 14, or 16; A is independently selected from the group consisting of: 2-[2-(2-Aminoethoxy)ethoxy]acetic Acid (Aeea), gamma linked glutamate (γE); y is 3; and z is 3.

Another embodiment of the present disclosure is a compound comprising a cationic moiety of Formula (I), wherein J is CH3, x is 14; A is independently selected from the group consisting of: 2-[2-(2-Aminoethoxy)ethoxy]acetic Acid (Aeea), gamma linked glutamate (γE); y is 3; and z is 3.

Another embodiment of the present disclosure is a compound comprising a cationic moiety of Formula (I), wherein J is CH3, x is 10, 12, 14, or 16; A is 2-[2-(2-Aminoethoxy)ethoxy]acetic Acid (Aeea); and z is 3.

Another embodiment of the present disclosure is a compound comprising a cationic moiety of Formula (I), wherein J is CH3, x is 14; A is 2-[2-(2-Aminoethoxy)ethoxy]acetic Acid (Aeea); and z is 3.

Another embodiment of the present disclosure is a compound comprising a cationic moiety of Formula (I), wherein J is CH3, x is 10, 12, 14, or 16; A is 2-[2-(2-Aminoethoxy)ethoxy]acetic Acid (Aeea); y is 3; and z is 3.

Another embodiment of the present disclosure is a compound comprising a cationic moiety of Formula (I), wherein J is CH3, x is 14; A is 2-[2-(2-Aminoethoxy)ethoxy]acetic Acid (Aeea); y is 3; and z is 3.

Another embodiment of the present disclosure is a compound comprising a cationic moiety of Formula (I), wherein J is CH3, x is 10, 12, 14, or 16; A is gamma amino butyric acid (γAbu); and z is 3.

Another embodiment of the present disclosure is a compound comprising a cationic moiety of Formula (I), wherein J is CH3, x is 14; A is gamma amino butyric acid (γAbu); and z is 3.

Another embodiment of the present disclosure is a compound comprising a cationic moiety of Formula (I), wherein J is CH3, x is 10, 12, 14, or 16; A is gamma amino butyric acid (γAbu); y is 3; and z is 3.

Another embodiment of the present disclosure is a compound comprising a cationic moiety of Formula (I), wherein J is CH3, x is 14; A is gamma amino butyric acid (γAbu); y is 3; and z is 3.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), and gamma amino butyric acid (γAbu); and z is 2.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), and gamma amino butyric acid (γAbu); and z is 2.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), and gamma amino butyric acid (γAbu); y is 3; and z is 2.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), and gamma amino butyric acid (γAbu); y is 3; and z is 2.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), gamma linked glutamate (γE); and z is 2.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), gamma linked glutamate (γE); and z is 2.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), gamma linked glutamate (γE); y is 3; and z is 2.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), gamma linked glutamate (γE); y is 3; and z is 2.

Another embodiment of the present disclosure is a compound comprising a cationic moiety of Formula (I), wherein J is HOOC, x is 10, 12, 14, or 16; A is 2-[2-(2-Aminoethoxy)ethoxy]acetic Acid (Aeea); and z is 2.

Another embodiment of the present disclosure is a compound comprising a cationic moiety of Formula (I), wherein J is HOOC, x is 14; A is 2-[2-(2-Aminoethoxy)ethoxy]acetic Acid (Aeea); and z is 2.

Another embodiment of the present disclosure is a compound comprising a cationic moiety of Formula (I), wherein J is HOOC, x is 10, 12, 14, or 16; A is 2-[2-(2-Aminoethoxy)ethoxy]acetic Acid (Aeea); y is 3; and z is 2.

Another embodiment of the present disclosure is a compound comprising a cationic moiety of Formula (I), wherein J is HOOC, x is 14; A is 2-[2-(2-Aminoethoxy)ethoxy]acetic Acid (Aeea); y is 3; and z is 2.

Another embodiment of the present disclosure is a compound comprising a cationic moiety of Formula (I), wherein J is HOOC, x is 10, 12, 14, or 16; A is gamma amino butyric acid (γAbu); and z is 2.

Another embodiment of the present disclosure is a compound comprising a cationic moiety of Formula (I), wherein J is HOOC, x is 14; A is gamma amino butyric acid (γAbu); and z is 2.

Another embodiment of the present disclosure is a compound comprising a cationic moiety of Formula (I), wherein J is HOOC, x is 10, 12, 14, or 16; A is gamma amino butyric acid (γAbu); y is 3; and z is 2.

Another embodiment of the present disclosure is a compound comprising a cationic moiety of Formula (I), wherein J is HOOC, x is 14; A is gamma amino butyric acid (γAbu); y is 3; and z is 2.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), and gamma amino butyric acid (γAbu); and z is 3.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), and gamma amino butyric acid (γAbu); and z is 3.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), and gamma amino butyric acid (γAbu); y is 3; and z is 3.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), and gamma amino butyric acid (γAbu); y is 3; and z is 3.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), gamma linked glutamate (γE); and z is 3.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), gamma linked glutamate (γE); and z is 3.

Another embodiment of the present disclosure is a compound comprising a cationic moiety of Formula (I), wherein J is HOOC, x is 10, 12, 14, or 16; A is independently selected from the group consisting of: 2-[2-(2-Aminoethoxy)ethoxy]acetic Acid (Aeea), gamma linked glutamate (γE); y is 3; and z is 3.

Another embodiment of the present disclosure is a compound comprising a cationic moiety of Formula (I), wherein J is HOOC, x is 14; A is independently selected from the group consisting of: 2-[2-(2-Aminoethoxy)ethoxy]acetic Acid (Aeea), gamma linked glutamate (γE); y is 3; and z is 3.

Another embodiment of the present disclosure is a compound comprising a cationic moiety of Formula (I), wherein J is HOOC, x is 10, 12, 14, or 16; A is 2-[2-(2-Aminoethoxy)ethoxy]acetic Acid (Aeea); and z is 3.

Another embodiment of the present disclosure is a compound comprising a cationic moiety of Formula (I), wherein J is HOOC, x is 14; A is 2-[2-(2-Aminoethoxy)ethoxy]acetic Acid (Aeea); and z is 3.

Another embodiment of the present disclosure is a compound comprising a cationic moiety of Formula (I), wherein J is HOOC, x is 10, 12, 14, or 16; A is 2-[2-(2-Aminoethoxy)ethoxy]acetic Acid (Aeea); y is 3; and z is 3.

Another embodiment of the present disclosure is a compound comprising a cationic moiety of Formula (I), wherein J is HOOC, x is 14; A is 2-[2-(2-Aminoethoxy)ethoxy]acetic Acid (Aeea); y is 3; and z is 3.

Another embodiment of the present disclosure is a compound comprising a cationic moiety of Formula (I), wherein J is HOOC, x is 10, 12, 14, or 16; A is gamma amino butyric acid (γAbu); and z is 3.

Another embodiment of the present disclosure is a compound comprising a cationic moiety of Formula (I), wherein J is HOOC, x is 14; A is gamma amino butyric acid (γAbu); and z is 3.

Another embodiment of the present disclosure is a compound comprising a cationic moiety of Formula (I), wherein J is HOOC, x is 10, 12, 14, or 16; A is gamma amino butyric acid (γAbu); y is 3; and z is 3.

Another embodiment of the present disclosure is a compound comprising a cationic moiety of Formula (I), wherein J is HOOC, x is 14; A is gamma amino butyric acid (γAbu); y is 3; and z is 3.

Another embodiment of the present disclosure is a compound comprising a cationic moiety of Formula (I), wherein J is CH3 or HOOC; x is 10, 12, 14, or 16; (A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3 or HOOC; x is 14; (A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3 or HOOC; x is 10, 12, 14, or 16; (A)y is Aeea-Aeea-Aeea, γAbu-γAbu-γAbu, γAbu-Aeea-Aeea, γE-Aeea-Aeea, or E-Aeea-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3 or HOOC; x is 14; (A)y is Aeea-Aeea-Aeea, γAbu-γAbu-γAbu, γAbu-Aeea-Aeea, γE-Aeea-Aeea, or E-Aeea-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3; x is 10, 12, 14, or 16; (A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3; x is 14; (A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3; x is 10, 12, 14, or 16; (A)y is Aeea-Aeea-Aeea, γAbu-γAbu-γAbu, γAbu-Aeea-Aeea, γE-Aeea-Aeea, or E-Aeea-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure is a compound comprising a cationic moiety of Formula (I), wherein J is CH3; x is 14; (A)y is Aeea-Aeea-Aeea, γAbu-γAbu-γAbu, γAbu-Aeea-Aeea, γE-Aeea-Aeea, or E-Aeea-Aeea; and (B)z is Dap-Dap, Dab-Dab, Dab-Dap, Dap-Dab, Dap-Dap-Dap, Dab-Dab-Dap, Dab-Dap-Dap, Dap-Dab-Dap, Dap-Dap-Dap-Dap, Dab-Dab-Dap-Dap, Dab-Dap-Dap-Dap, or Dap-Dab-Dap-Dap.

Another embodiment of the present disclosure, the cationic moiety of Formula (I) is any one of [SEQ ID. NO: 10 to 22 (See Table 1 in Example 1) or 51 to 69], wherein SEQ. ID. NO: 10 to 22 and 51-69 are as follows, CH3(CH2)14(C═O)-Aeea-Aeea-Aeea-Dab-Dab [SEQ ID NO: 10], CH3(CH2)14(C═O)-γAbu-γAbu-Dab-Dab [SEQ ID NO: 11], CH3(CH2)14(C═O)-γAbu-γAbu-γAbu-Dab-Dab [SEQ ID NO: 12], CH3(CH2)14(C═O)-Aeea-Aeea-Dap-Dab [SEQ ID NO: 13], CH3(CH2)14(C═O)-Aeea-Aeea-Aeea-Dap-Dap [SEQ ID NO: 14], CH3(CH2)14(C═O)-γE-Aeea-Aeea-Dab-Dab [SEQ ID NO: 15], CH3(CH2)14(C═O)-E-Aeea-Aeea-Dab-Dab [SEQ ID NO: 16], CH3(CH2)14(C═O)-Aeea-Aeea-Aeea-Dab-Dap [SEQ ID NO: 17], (HOOC)(CH2)16(C═O)-Aeea-Aeea-Aeea-Dab-Dab [SEQ ID NO: 18], CH3(CH2)14(C═O)-Aeea-Aeea-Aeea-Dap-Dap-Dap [SEQ ID NO: 19], (HOOC)(CH2)16(C═O)-Aeea-Aeea-Aeea-Dap-Dap [SEQ ID NO: 20], (HOOC)(CH2)16(C═O)-Aeea-Aeea-Dab-Dab [SEQ ID NO: 21], CH3(CH2)14(C═O)-Aeea-Aeea-Aeea-Dap-Dap-Dap-Dap [SEQ ID NO: 22], CH3(CH2)14(C═O)-Aeea-Aeea-Aeea-Dap-Dab [SEQ ID NO: 51], CH3(CH2)14(C═O)-γAbu-γAbu-γAbu-Dap-Dap [SEQ ID NO: 52], CH3(CH2)14(C═O)-γAbu-γAbu-γAbu-Dap-Dab [SEQ ID NO: 53], CH3(CH2)14(C═O)-γAbu-γAbu-γAbu-Dab-Dap [SEQ ID NO: 54], CH3(CH2)14(C═O)-γE-Aeea-Aeea-Dap-Dap [SEQ ID NO: 55], CH3(CH2)14(C═O)-γE-Aeea-Aeea-Dap-Dab [SEQ ID NO: 56], CH3(CH2)14(C═O)-γE-Aeea-Aeea-Dab-Dap [SEQ ID NO: 57], (HOOC)(CH2)14(C═O)-Aeea-Aeea-Aeea-Dap-Dap [SEQ ID NO: 58], (HOOC)(CH2)14(C═O)-Aeea-Aeea-Aeea-Dab-Dab [SEQ ID NO: 59], (HOOC)(CH2)14(C═O)-Aeea-Aeea-Aeea-Dap-Dab [SEQ ID NO: 60], (HOOC)(CH2)14(C═O)-Aeea-Aeea-Aeea-Dab-Dap [SEQ ID NO: 61], (HOOC)(CH2)14(C═O)-γAbu-γAbu-γAbu-Dap-Dap [SEQ ID NO: 62], (HOOC)(CH2)14(C═O)-γAbu-γAbu-γAbu-Dab-Dab [SEQ ID NO: 63], (HOOC)(CH2)14(C═O)-γAbu-γAbu-γAbu-Dap-Dab [SEQ ID NO: 64], (HOOC)(CH2)14(C═O)-γAbu-γAbu-γAbu-Dab-Dap [SEQ ID NO: 65], (HOOC)(CH2)14(C═O)-γE-Aeea-Aeea-Dap-Dap [SEQ ID NO: 66], (HOOC)(CH2)14(C═O)-γE-Aeea-Aeea-Dab-Dab [SEQ ID NO: 67], (HOOC)(CH2)14(C═O)-γE-Aeea-Aeea-Dap-Dab [SEQ ID NO: 68], or (HOOC)(CH2)14(C═O)-γE-Aeea-Aeea-Dab-Dap [SEQ ID NO: 69].

Another embodiment of the present disclosure is a composition of Formula (I), for use in the manufacture of a medicament, such as a medical composition.

Another embodiment of the present disclosure is a composition (e.g., a pharmaceutical composition) comprising a compound that comprises an alkyl cationic moiety of Formula (I) (e.g., conjugated to a peptide) and one or more pharmaceutically acceptable carriers and/or excipients.

Another embodiment of the present disclosure is a composition comprising a compound that comprises a cationic moiety of Formula (I) further comprising of an immune modulator or anti-cancer agent formulation.

### Formula (II)

Another embodiment of the present disclosure is a compound comprising a cationic moiety of Formula (I), further comprising a covalently linked peptide to give a conjugated peptide of Formula (II):

J-(CH2)x(CO)-(A)y-(B)z-Peptide,
wherein the portion corresponding to Formula (I) is covalently linked to the peptide via amide bond to Dap or Dab; wherein the variables within the portion corresponding to Formula (I) are defined to be the same as in Formula (I), and wherein the conjugated peptide of Formula (II) has: i) more biological activity than the unmodified peptide at the equivalent bolus dose (mole/kg); ii) longer in vivo half-life and/or higher levels in the blood over time after a bolus administration compared to the unmodified peptide while retaining its activity; and/or iii) no toxicity or ataxia at a bolus dose of 3.0 μmol/kg or less in rats.

The compound of Formula II, wherein the compound binds to a natriuretic peptide receptor, and wherein Formula (II) has no adverse effect or ataxia at a bolus dose of 3.0 μmol/kg and lower in rats.

Another embodiment of Formula (II), the cationic alkyl moiety of Formula (I) is covalently linked to the N-terminal of the peptide or a side chain amino group (pendant amino) on the peptide.

Another embodiment of Formula (II), the cationic alkyl moiety of Formula (I) is covalently linked to the N-terminal of the peptide.

In one embodiment of Formula (II), the cationic moiety of Formula (I) is selected from [SEQ ID NOs: 10 to 22 and 51 to 69]

In one embodiment of Formula (II), the peptide portion is a natriuretic peptide or a natriuretic peptide derivative. As used herein, “derivative” as it applies to a peptide means a native peptide modified by replacing one of more amino acids, adding one or more amino acids, removing one or more amino acids, or any combinations thereof while retaining the biological activity of the native form of the peptide.

In one embodiment of Formula (II), the peptide portion is a natriuretic peptide [SEQ ID NOs: 32, 44, 48, 75] or a natriuretic peptide derivative, wherein one or more methionine residue(s) in the natriuretic peptide are replaced by glutamine (Q), Norleucine (Nle), methoxinine (Mox), or Lucine (L).

In one embodiment of Formula (II), the peptide portion is a natriuretic peptide [SEQ ID NOs: 32, 44, 48, 75] or a natriuretic peptide derivative, wherein one or more methionine residue(s) in the natriuretic peptide are replaced by glutamine (Q), Norleucine (Nle), methoxinine (Mox), or Lucine (L) and the cationic alkyl moiety of Formula (I) is selected from [SEQ ID NOs: 10 to 22 and 51 to 69].

In one embodiment of Formula (II), the peptide is a natriuretic peptide derivative, wherein one or more methionine residue(s) in a natriuretic peptide is(are) replaced by glutamine (Q), Norleucine (Nle), methoxinine (Mox), or Lucine (L) and the moiety of Formula (I) is selected from [SEQ ID NOs: 10 to 22 and 51 to 69].

In one embodiment of Formula (II), the peptide is a natriuretic peptide derivative, wherein one or more methionine residue(s) in a natriuretic peptide are replaced by glutamine (Q), Norleucine (Nle), methoxinine (Mox), or Lucine (L) and the moiety of Formula (I) is selected from [SEQ ID NO: 10 to 22].

In one embodiment of Formula (II), the peptide binds to natriuretic peptide receptor B (NPRB), natriuretic peptide receptor C (NPRC), or both NPRB and NPRC.

In one embodiment of Formula (II), the peptide binds to natriuretic peptide receptor A (NPRA), natriuretic peptide receptor B (NPRB), and/or natriuretic peptide receptor C (NPRC).

In one embodiment of Formula (II), the peptide is a natriuretic peptide receptor B (NPRB) agonist.

In one embodiment of Formula (II), the peptide is a natriuretic peptide receptor C (NPRC) agonist.

In one embodiment of Formula (II), the peptide is a natriuretic peptide receptor A (NPRA) agonist.

In one embodiment of Formula (II), the peptide generates a physiological effect. Non-limiting examples of physiological effects that may be generated by the peptide include: antiproliferative effects, decreased endothelial permeability, inhibition of cyclooxygenase 2 (COX-2) expression, decreasing blood pressure, antagonizing the renin-angiotensin-aldosterone system, inhibiting cardiac hypertrophy, prolonged increase in blood cGMP, changes in cAMP, increased survival from Sepsis, increased survival from Acute Lung Injury, increased survival from Acute Respiratory Distress Syndrome, decrease in MPO positive cells, decrease in number of cells in Alveolar fluid or in Bronchoalveolar Lavage fluid, decrease in amount of protein in Alveolar fluid or Bronchoalveolar Lavage fluid, decrease in lung weight per body weight, decrease in Monocyte Chemoattractant Protein-1, decrease in IL-6, decrease TNF-alpha, decrease in A1008/A9, decrease fibrosis, decrease in tumor volume, decrease inflammation, and/or decrease cancer burden.

In one embodiment of Formula (II), the peptide is a natriuretic peptide derivative, wherein one or more methionine residue(s) in a natriuretic peptide are replaced by glutamine (Q).

In one embodiment of Formula (II), the conjugated peptide is defined by SEQ ID NOs: 29-31, 33-43, 45-47, 49, 50.

In one embodiment of Formula (II), the conjugated peptide is defined by SEQ ID NOs: 29-31, 33-43.

In one embodiment of Formula (II), the conjugated peptide is defined by SEQ ID NOs: 29-31.

In one embodiment of Formula (II), the conjugated peptide is defined by SEQ ID NOs: 29.

In one embodiment of Formula (II), the conjugated peptide is defined by SEQ ID NOs: 30.

In one embodiment of Formula (II), the conjugated peptide is defined by SEQ ID NOs: 31.

Compound comprising a cationic moiety of Formula (I) and/or a conjugated peptide of Formula (II) for use in the manufacture of medical composition and/or for use in the treatment of diseases.

The use of a compound or composition of the present disclosure is effective in resolving alveolar inflammatory edema, improving blood oxygenation, and/or improving survival, as evident from the presented examples. Additionally, a compound or composition of the present disclosure is effective in preventing and resolving pulmonary fibrosis.

Another embodiment of the present invention is a compound comprising a cationic alkyl moiety or conjugated peptide of Formula (I) or Formula (II) for use in the manufacture (or for use in a method of manufacture) of a medicament (or medical composition).

Another embodiment of the present invention is the compound comprising a cationic alkyl moiety of Formula (I) for use as an excipient in the manufacture of a medical composition.

The compound of Formula II, for use in the manufacture of a medical composition, by the addition of one or more pharmaceutically acceptable carriers or excipients, such as, for example, a bulking agent (e.g., sugar), a buffering agent, a stabilizer (e.g. cyclodextrin), and/or a preservative.

The compound of Formula II, for use in treating a disease or condition in a subject in need thereof, by administering to the subject a therapeutically effective bolus dose of 10.0 μmol/kg or lower, and/or between 10.0 μmol/kg and 0.0001 μmol/kg inclusive

Another embodiment of the present invention is a medical compound comprising a cationic moiety or conjugated peptide of Formula (I) and/or Formula (II), further comprising one or more pharmaceutically acceptable excipients.

Another embodiment of the present invention is the compound comprising a conjugated peptide of Formula (II) for use in: a) the manufacture of a medical composition or b) the treatment of a disease in a subject.

Another embodiment of the present invention provides a compound comprising a cationic moiety or conjugated peptide of Formula (I) and/or Formula (II) for use in the manufacture (or for use in a method of manufacture) of medicaments (or medical compositions) for the treatment of diseases affecting the lung, liver, heart, bones and/or joints, kidneys, prostate, brain, eye, skin, muscle, blood, gastrointestinal track, bladder, testis, ovary, uterus, and/or blood vessel.

The present disclosure also provides compounds comprising a cationic moiety or conjugated peptide of Formula (I) and/or Formula (II) for use in a method of treatment for disease(s) affecting the lung, liver, heart, bone/joint, kidney, prostate, brain, eye, skin, muscle, blood, gastrointestinal track, bladder, testis, ovary, uterus, and/or blood vessel, wherein the method comprises parenterally administering a pharmaceutical composition comprising a compound comprising a cationic moiety or conjugated peptide of Formula (I) and/or Formula (II) at a therapeutically effective bolus dose of 3.0 μmol/kg or less.

Another embodiment of the present invention provides conjugated peptides of Formula (II) for use in the manufacture (or for use in a method of manufacture) of medicaments (or medical compositions) for the treatment of diseases affecting the lung, liver, heart, bone/joint, kidney, prostate, brain, eye, skin, muscle, blood, gastrointestinal track, bladder, testis, ovary, uterus, and/or blood vessel, wherein the method comprises adding one or more pharmaceutically acceptable excipients to the conjugated peptide Formula (II).

In some embodiments, the medicament or medical composition for the treatment of diseases affecting the lung treats a disease selected from ALI, ARDS, COVID (e.g., COVID-19), inflammation, sepsis, fibrosis, or cancer. In some embodiments, the medicament or medical composition for the treatment of diseases affecting the liver treats a disease selected from non-alcoholic fatty liver disease (NAFLD), non-alcoholic steatohepatitis (NASH), inflammation, fibrosis, or cancer. In some embodiments, the medicament or medical composition for the treatment of diseases affecting the heart treats a disease selected from heart failure with preserved ejection fraction (HFpEF), heart failure with reduced ejection fraction (HFrEF), acute heart failure, or congestive heart failure. In some embodiments, the medicament or medical composition for the treatment of diseases affecting the bones and/or joints treats a disease selected from osteoporosis, osteoarthritis, rheumatoid arthritis, inflammation cancer, or dwarfism. In some embodiments, the medicament or medical composition for the treatment of diseases affecting the kidneys treats a disease selected from chronic kidney disease (CKD), acute kidney injury (AKI), drug induced kidney injury, inflammation/nephritis, kidney fibrosis, glomerulosclerosis, or kidney cancer. In some embodiments, the medicament or medical composition for the treatment of diseases affecting the prostate treats a disease selected from prostate hyperplasia or prostate cancer.

Another embodiment of the present disclosure relates to a medical composition comprising a conjugated peptide of Formula (II) for use in the treatment (or use in a method of treatment) of diseases affecting the lung, liver, heart, bone/joint, kidney, prostate, brain, eye, skin, muscle, blood, gastrointestinal track, bladder, testis, ovary, uterus, and/or blood vessel, wherein the treatment comprises administering the compound comprising a conjugated peptide of Formula (II) to a subject in need thereof at a therapeutically effective bolus dose of 3.0 μmol/kg or lower, wherein the peptide portion of the conjugated peptide of Formula (II) is a natriuretic peptide or derivative thereof, and the portion corresponding to a cationic alkyl moiety of Formula (I) is selected from SEQ ID NOs: 10 to 22 and 51 to 69.

In some embodiments, the present disclosure provides use of a conjugated peptide of Formula II for the treatment of a disease, condition or disorder. In some embodiments, the disease, condition or disorder affects the lung (e.g., a disease selected from ALI, ARDS, COVID (e.g., COVID-19), inflammation, sepsis, fibrosis, or lung cancer). In some embodiments, the disease, condition or disorder affects the liver (e.g., a disease selected from non-alcoholic fatty liver disease (NAFLD), non-alcoholic steatohepatitis (NASH), inflammation, fibrosis, or cancer. In some embodiments, the disease, condition or disorder affects the heart (e.g., a disease selected from heart failure with preserved ejection fraction (HFpEF), heart failure with reduced ejection fraction (HFrEF), acute heart failure, or congestive heart failure). In some embodiments, the disease, condition or disorder affects the bones and/or joints (e.g., a disease selected from osteoporosis, osteoarthritis, rheumatoid arthritis, inflammation cancer, or dwarfism). In some embodiments, the disease, condition or disorder affects the kidneys (e.g., a disease selected from chronic kidney disease (CKD), acute kidney injury (AKI), drug induced kidney injury, inflammation/nephritis, kidney fibrosis, glomerulosclerosis, or kidney cancer). In some embodiments, the disease, condition or disorder affects the prostate, such as, for example, prostate hyperplasia or prostate cancer.

Another embodiment of the present disclosure relates to a medical composition comprising a conjugated peptide of Formula (II) for use in the treatment (or use in a method of treatment) of ALI, ARDS, COVID, sepsis, and/or fibrosis wherein the treatment comprises administering a compound comprising a conjugated peptide of Formula (II) to a subject in need thereof at a therapeutically effective bolus dose of 3.0 μmol/kg or lower, wherein the peptide portion of the conjugated peptide of Formula (II) is a natriuretic peptide [e.g. SEQ ID NOs: 32, 44, 48, 75] or derivative thereof wherein one or more methionine residues in the natriuretic peptide are replaced by glutamine (Q), Norleucine (Nle), methoxinine (Mox), or Lucine (L) and the cationic alkyl moiety of Formula (I) portion is [SEQ ID NOs: 10 to 22 and 51 to 69].

Another embodiment of the present disclosure provides a conjugated peptide of Formula (II) for use in the manufacture (or for use in a method of manufacture) of medicaments (or medical compositions) for the treatment of ALI, ARDS, COVID, sepsis, and/or lung fibrosis, comprising adding one or more pharmaceutically acceptable excipients to the conjugated peptide of Formula (II), wherein the conjugated peptide of Formula (II) is selected from SEQ ID NOs: 29-31, 33-43, 45-47, 49-50.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering to a subject in need thereof a therapeutically effective bolus dose of 3.0 μmol/kg or lower of the conjugated peptide of Formula (II), wherein the conjugated peptide of Formula (II) is selected from SEQ ID NOs: 29-31, 33-43, 45-47, 49-50.

Another embodiment of the present disclosure provides a conjugated peptide of Formula (II) for use in the manufacture (or for use in a method of manufacture) of medicaments (or medical compositions) for the treatment of ALI, ARDS, COVID, sepsis, and/or lung fibrosis, wherein the manufacture or method of manufacture comprises adding one or more pharmaceutically acceptable excipients to the conjugated peptide of Formula (II), wherein the conjugated peptide of Formula (II) is selected from SEQ ID NOs: 29-31, 33-43.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering the conjugated peptide of Formula (II) to a subject in need thereof at a therapeutically effective bolus dose of 3.0 μmol/kg or lower, wherein the conjugated peptide of Formula (II) is selected from SEQ ID NOs: 29-31, 33-43.

Another embodiment of the present disclosure provides a conjugated peptide of Formula (II) for use in the manufacture (or for use in a method of manufacture) of medicaments (or medical compositions) for the treatment of ALI, ARDS, COVID, sepsis, and/or lung fibrosis, wherein the manufacture or method of manufacture comprises adding one or more pharmaceutically acceptable excipients to the conjugated peptide of Formula (II), wherein the conjugated peptide of Formula (II) is selected from SEQ ID NOs: 29-31.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering a conjugated peptide of Formula (II) to a subject in need thereof at a therapeutically effective bolus dose of 3.0 μmol/kg or lower, wherein the conjugated peptide of Formula (II) is selected from SEQ ID NOs: 29-31.

Another embodiment of the present disclosure provides a conjugated peptide of Formula (II) for use in the manufacture (or for use in a method to manufacture) of medicaments (or medical compositions) for the treatment of ALI, ARDS, COVID, sepsis, and/or lung fibrosis, wherein the treatment comprises adding one or more pharmaceutically acceptable excipients to the conjugated peptide of Formula (II), wherein the conjugated peptide of Formula (II) is SEQ ID NO: 29.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO: 29.

Another embodiment of the present disclosure provides a conjugated peptide of Formula (II) for use in the manufacture (or for use in a method to manufacture) of medicaments (or medical compositions) for the treatment of ALI, ARDS, COVID, sepsis, and/or lung fibrosis, wherein the manufacture or method comprises adding one or more pharmaceutically acceptable excipients to the conjugated peptide of Formula (II), wherein the conjugated peptide of Formula (II) is SEQ ID NO: 30.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering to a subject in need thereof the conjugated peptide of Formula (II) at a therapeutically effective bolus dose of 3.0 μmol/kg or lower, wherein the conjugated peptide of Formula (II) is SEQ ID NO: 30.

Another embodiment of the present disclosure provides a conjugated peptide of Formula (II) for use in the manufacture (or for use in a method to manufacture) of medicaments (or medical compositions) for the treatment of ALI, ARDS, COVID, sepsis, and/or lung fibrosis, wherein the manufacture or method comprises adding one or more pharmaceutically acceptable excipients to the conjugated peptide of Formula (II), wherein the conjugated peptide of Formula (II) is SEQ ID NO: 31.

Another embodiment of the present disclosure relates to a medical composition comprising a conjugated peptide of Formula (II) for use in the treatment (or use in a method of treatment) of ALI, ARDS, COVID, sepsis, and/or fibrosis, wherein the treatment comprises administering the conjugated peptide of Formula (II) to a subject in need thereof at a therapeutically effective bolus dose of 3.0 μmol/kg or lower), wherein the conjugated peptide of Formula (II) is SEQ ID NO: 31.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, the treatment comprising administering the conjugated peptide of Formula (II) to a subject in need thereof at a therapeutically effective bolus dose of 3.0 μmol/kg or lower, wherein the peptide portion of the conjugated peptide of Formula (II) is a natriuretic peptide (e.g. SEQ ID NO: 32, 44, 48, 75) or derivative thereof, wherein one or more methionine residue(s) in the natriuretic peptide are replaced by glutamine (Q), Norleucine (Nle), methoxinine (Mox), or Lucine (L), and the portion corresponding to a cationic alkyl moiety of Formula (I) is selected from SEQ ID NO: 10 to 22 and 51 to 69.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, wherein the manufacture or method comprises adding one or more pharmaceutically acceptable excipients to the conjugated peptide of Formula (II), wherein the conjugated peptide of Formula (II) is selected from SEQ ID NO: 29-31, 33-43, 45-47, and 49-50.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, the treatment comprising administering the conjugated peptide of Formula (II) to a subject in need thereof at a therapeutically effective bolus dose of 3.0 μmol/kg or lower), wherein the conjugated peptide of Formula (II) is selected from SEQ ID NO: 29-31, 33-43, 45-47, and 49-50.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, the manufacture or method comprising adding one or more pharmaceutically acceptable excipients to the conjugated peptide of Formula (II), wherein the conjugated peptide of Formula (II) is selected from SEQ ID NOs: 29-31, and 33-43.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, wherein the treatment comprises administering to a subject in need thereof the conjugated peptide of Formula (II) at a therapeutically effective bolus dose of 3.0 μmol/kg or lower), wherein the conjugated peptide of Formula (II) is selected from SEQ ID NOs: 29-31, and 33-43.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, wherein the manufacture or method comprises adding one or more pharmaceutically acceptable excipients to the conjugated peptide of Formula (II), wherein the conjugated peptide of Formula (II) is selected from SEQ ID NO:: 29-31.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, wherein the treatment comprises administering the conjugated peptide of Formula (II) to a subject in need thereof at a therapeutically effective bolus dose of 3.0 μmol/kg or lower, wherein the conjugated peptide of Formula (II) is selected from SEQ ID NO:: 29-31.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, wherein the manufacture or method comprises adding one or more pharmaceutically acceptable excipients to the conjugated peptide of Formula (II), wherein the conjugated peptide of Formula (II) is SEQ ID NO:: 29.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, wherein the treatment comprises administering to a subject in need thereof the conjugated peptide of Formula (II) at a therapeutically effective bolus dose of 3.0 μmol/kg or lower, wherein the conjugated peptide of Formula (II) is SEQ ID NO:: 29.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, the manufacture or method comprising adding one or more pharmaceutically acceptable excipients to the conjugated peptide of Formula (II), wherein the conjugated peptide of Formula (II) is SEQ ID NO:: 30.

Another embodiment of the present disclosure relates to a medical composition comprising a conjugated peptide of Formula (II) for use in the treatment (or use in a method of treatment) of one or a combination of medical condition selected from low blood oxygenation, elevated levels of inflammatory cells in the lung, pulmonary edema, sepsis, and/or bacteremia, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 30.

Another embodiment of the present disclosure provides a conjugated peptide of Formula (II) for use in the manufacture (or for use in a method to manufacture) of medicaments (or medical compositions) for the treatment of one or a combination of medical conditions selected from low blood oxygenation, elevated levels of inflammatory cells in the lung, pulmonary edema, sepsis, and/or bacteremia, wherein the manufacture or method comprises adding one or more pharmaceutically acceptable excipients to the conjugated peptide of Formula (II), wherein the conjugated peptide of Formula (II) is SEQ ID NO:: 31.

Another embodiment of the present disclosure relates to a medical composition comprising a conjugated peptide of Formula (II) for use in the treatment (or use in a method of treatment) of one or a combination of medical conditions selected from low blood oxygenation, elevated levels of inflammatory cells in the lung, pulmonary edema, sepsis, and/or bacteremia, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 31.

Another embodiment of the present disclosure relates to a medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a therapeutically effective bolus dose of 3.0 μmol/kg or lower, wherein the peptide portion of the conjugated peptide of Formula (II) is a natriuretic peptide (e.g. SEQ ID NO:: 32, 44, 48, 75) or derivative thereof, wherein one or more methionine residue(s) in the natriuretic peptide are replaced by glutamine (Q), Norleucine (Nle), methoxinine (Mox), or Lucine (L), and the portion of the conjugated peptide corresponding to a cationic alkyl moiety of Formula (I) is SEQ ID NO:: 10 to 22 and 51 to 69.

Another embodiment of the present disclosure provides a conjugated peptide of Formula (II) for use in the manufacture (or for use in a method to manufacture) of medicaments (or medical compositions) for the treatment of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipient to the conjugated peptide of Formula (II), wherein the conjugated peptide of Formula (II) is selected from SEQ ID NO:: 29-31, 33-43, 45-47, 49-50.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a bolus dose of 3.0 μmol/kg or lower, wherein the conjugated peptide of Formula (II) is selected from SEQ ID NO:: 29-31, 33-43, 45-47, 49-50.

Another embodiment of the present disclosure provides a conjugated peptide of Formula (II) for use in the manufacture (or for use in a method to manufacture) of medicaments (or medical compositions) for the treatment of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipients to the conjugated peptide of Formula (II), wherein the conjugated peptide of Formula (II) is selected from SEQ ID NO:: 29-31, 33-43.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a bolus dose of 3.0 μmol/kg or lower, wherein the conjugated peptide of Formula (II) is selected from SEQ ID NO:: 29-31, 33-43.

Another embodiment of the present disclosure provides a conjugated peptide of Formula (II) for use in the manufacture (or for use in a method to manufacture) of medicaments (or medical compositions) for the treatment of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipients to the conjugated peptide of Formula (II), wherein the conjugated peptide of Formula (II) is SEQ ID NO:: 29-31.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 29-31.

Another embodiment of the present disclosure provides a conjugated peptide of Formula (II) for use in the manufacture (or for use in a method to manufacture) of medicaments (or medical compositions) for the treatment of metastatic cancer located in any one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipients to the conjugated peptide of Formula (II), wherein the conjugated peptide of Formula (II) is SEQ ID NO:: 29.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 29.

Another embodiment of the present disclosure provides a conjugated peptide of Formula (II) for use in the manufacture (or for use in a method to manufacture) of medicaments (or medical compositions) for the treatment of metastatic cancer located in any one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipients to the conjugated peptide of Formula (II), wherein the conjugated peptide of Formula (II) is SEQ ID NO:: 30.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 30.

Another embodiment of the present disclosure provides a conjugated peptide of Formula (II) for use in the manufacture (or for use in a method to manufacture) of medicaments (or medical compositions) for the treatment of metastatic cancer located in any one or more organs selected from lung, liver, heart, bone/joint, kidney, prostate, brain, eye, skin, muscle, blood, gastrointestinal track, bladder, prostate, testis, ovary, and/or uterus, wherein the manufacture or method comprises adding one or more pharmaceutically acceptable excipients to the conjugated peptide of Formula (II), wherein the conjugated peptide of Formula (II) is SEQ ID NO:: 31.

Another embodiment of the present disclosure relates to the medical composition comprising a conjugated peptide of Formula (II) for use in the treatment (or use in a method of treatment) of metastatic cancer located in one or more organs selected from lung, liver, heart, bone/joint, kidney, prostate, brain, eye, skin, muscle, blood, gastrointestinal track, bladder, prostate, testis, ovary, and/or uterus, wherein the treatment comprises administering the conjugated peptide of Formula (II) to a subject in need thereof at a therapeutically effective bolus dose of 3.0 μmol/kg or lower, wherein the conjugated peptide of Formula (II) is SEQ ID NO:: 31.

### Definitions

The following list of definitions clearly defines the invention in the present disclosure. Any terms that are not listed here but are present in the disclosure have the same meaning as would be understood among those skilled in the art.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms. For the purpose of the present disclosure the Alkyl group moiety is written using the formula CH3(CH2)x-CO—, HOOC(CH2)x-CO—, or —(CH2)x-, where x represents the number of methylene groups (i.e. CH2) that make up the alkyl chain and the CO represents the carbonyl group that links the alkyl moiety to the rest of the molecule. At various places in the present specification, substituents of compounds of the disclosure are disclosed in groups or in ranges. It is specifically intended that the disclosure include each and every individual sub-combination of the members of such groups and ranges. For example, the term “—(CH2)x-” where “x is 10-18” is specifically intended to individually disclose, but not be limited to “CH2—CH2—CH2—CH2—CH2—CH2—CH2—CH2—CH2—CH2”, “CH2—CH2—CH2—CH2—CH2—CH2—CH2—CH2—CH2—CH2—CH2—CH2”, “CH2—CH2—CH2—CH2—CH2—CH2—CH2—CH2—CH2—CH2—CH2—CH2—CH2—CH2”, “CH2—CH2—CH2—CH2—CH2—CH2—CH2—CH2—CH2—CH2—CH2—CH2—CH2—CH2—CH2—CH2”, and “CH2—CH2—CH2—CH2—CH2—CH2—CH2—CH2—CH2—CH2—CH2—CH2—CH2—CH2—CH2—CH2—CH2—CH2”.

As used herein, “Cationic alkyl” “cationic alkyl group” or “cationic alkyl moiety” is an alkyl group derived from a fatty acid covalently attached to one or more positively charged group(s) or moiety(ies) by a spacer or linker. The spacer or linker is made up of several amino acids in a chain and each amino acid may be unnatural (meaning not normally found in living higher organisms such as the D-form) or natural (meaning normally found in living organisms). The linker may be derived from an amino acid such as 2-[2-(2-(aminoethoxy)ethoxy]acetic acid (also known as 8-amino-3 6-dioxaoctanoic acid), gamma aminobutyrate (γ Abu), or naturally occurring amino acids such as glutamate (E) or gamma-linked glutamate (γE) which is a glutamic acid residue wherein the sidechain carboxyl (gamma, γ) is the moiety used to link to the nitrogen of the molecule or peptide to which it is bound instead of the typical alpha carboxyl. The positively charged group(s) are provided by amino acids with positively charged sidechains comprising an amine group. As used herein, the term “fatty acid” refers to a molecule having a carboxylic acid with a long alkyl chain (or aliphatic chain), which is either saturated or unsaturated. The carboxylic acid portion is a reactive moiety that can form an “acyl amide bond” with the linker.

As used herein, the term “acyl amide” or “fatty acid amide” refers to an alkyl chain (saturated or unsaturated) having a —C(O)NH— moiety at one or both ends. For example, in “—(CH2)x-CONH—” where —(CH2)x- is the alkyl portion, —CONH— is the amide portion, such that the “—(CH2)x-CONH—” is an acyl amide or fatty acid amide.
the phrase modifies is, within the scope of sound medical judgment, suitable for use in contact with the tissues of humans and lower animals without undue toxicity (ataxia), irritation, allergic response and the like, and are commensurate with a reasonable benefit/risk ratio.

As used herein, “allometric scaling” is a tool that drug developers use to predict human pharmacokinetics based upon animal data. Prediction methods like allometric scaling provide a “sneak peek” at how a drug might behave in humans before any clinical studies are conducted. This is important information for both drug developers and regulators (like the FDA) because it provides a data-driven foundation for establishing a safe starting bolus dose in humans. For the purpose of the present disclosure, the exponent used in allometric scaling is 0.7 (i.e., dose in other species=rat dose/((rat weight/weight of average other species)exp 0.7)). A 2.0 mg/kg dose in mice is equivalent to about 1.0 mg/kg rat dose, about 0.33 mg/kg dog dose, and 0.17 mg/kg human dose. In terms of mole mole dose, the 3.0 μmol/kg in rat is interpreted as 6.0 μmol/kg in mice, 1.0 μmol/kg in dog and 0.5 μmol/kg in human. A 5.0 μmol/kg in rat is interpreted as 10 μmol/kg in mice, 1.66 μmol/kg in dog and 0.83 μmol/kg in human. A 10 μmol/kg in rat is interpreted as 20 μmol/kg in mice, 3.33 μmol/kg in dog and 1.66 μmol/kg in human. For the purpose of the present specification the claimed doses are derived from rats, and it should be understood that it applies to other species after proper allometric scaling.

As used herein, a “bolus”, “bolus dose” or “bolus administration” refers to a single dose of a drug or other substance given or administered over a short period of time, for example, less than 10 minutes (e.g. less than 8 minutes, less than 5 minutes, less than 3 minutes, or less than 1 minute). Administration includes one of: injection in any part of the body (including but not limited to blood vessels, subcutaneous, intrathecal, or intradermal), enterally (e.g. orally, as a dosage form), inhalation (e.g., by intratracheal inhalation administration, where a subject is exposed to high aerosol concentrations so that the active pharmaceutical ingredient is deposited directly in the lower respiratory tract), or nasally (e.g., as an aerosol, liquid, or powder). For the purpose of the present disclosure, a bolus administration is distinguished from an infusion administration that usually takes 30 minutes or longer to complete.

As used herein, the terms “acute lung injury” or “ALI” and the more severe “acute respiratory distress syndrome” or “ARDS” are the pulmonary manifestations of an acute systemic inflammatory process characterized clinically by pulmonary infiltrates, hypoxemia and edema with no evidence of left atrial hypertension (see, e.g., Bernard, G. R., et al., J. Crit. Care, 1994. 9(1): p. 72-81; Rubenfeld, G. D., et al., N Engl J Med, 2005. 353(16): p. 1685-93; Brun-Buisson, C., et al., Intensive Care Med, 2004. 30(1): p. 51-61; and Phua, J., et al., Am J Respir Crit Care Med, 2009. 179(3): p. 220-7). ALI and ARDS are the acute onset of severe arterial hypoxemia (low oxygen level in the blood due to abnormal ventilation) with a PaO2/FiO2 of less than less than 300 Torr and less than or equal to 200 Torr, respectively. The signs and symptoms of ALI and ARDS often begin within two hours of an inciting event but have been known to take as long as 1-3 days; diagnostic criteria require a known insult to have happened within 7 days of the syndrome. Signs and symptoms may include shortness of breath, fast breathing, muscle fatigue and general weakness, low blood pressure, a dry hacking cough, and fever. ARDS is an overwhelming pulmonary inflammatory response to certain primary and secondary noxious stimuli such as pneumonia (e.g., aseptic pneumonia, viral pneumonia, bacterial pneumonia), sepsis, aspiration, inhalation injuries, near drowning, and pulmonary resection surgery (see, e.g., Alam, N., et al., Ann Thorac Surg, 2007. 84(4): p. 1085-91). ARDS is characterized by rapid-onset respiratory failure necessitating hospitalization and ventilatory support in an intensive care unit (ICU). If a patient survives ALI/ARDS, the long-term quality of life of the patient is often adversely affected due to lung scarring (see, e.g., Rubenfeld, G. D., et al., N Engl J Med, 2005. 353(16): p. 1685-93; Dowdy, D. W., et al., Intensive Care Med, 2006. 32(8): p. 1115-24). To date, no effective agent to treat acute lung injury (ALI) and ARDS has been identified, and there is a significant need for such an agent.

As used herein, “pneumonia” is an infection that inflames the air sacs in one or both lungs. The air sacs may fill with fluid or pus (purulent material), causing cough with phlegm or pus, fever, chills, and difficulty breathing.

As used herein, “COVID” or “corona virus induced disease” is a generic term for a disease caused by a corona virus. Coronavirus is a family of viruses of many different kinds, and some cause disease or COVID. For example, a coronavirus 19 (COVID 19) is identified in 2019 to cause COVID affecting the lung and other parts of the body and called is called COVID-19. lungs.

As used herein, the term “cancer” refers to a malignant tissue mass. Malignant tumor cells can “metastasize” (i.e., spread) to various organs in the body such as lung, liver, heart, bone/joint, kidney, prostate, brain, eye, skin, muscle, blood, gastrointestinal track, bladder, prostate, testis, ovary, and uterus through the blood and lymphatic system. The organs that are affected become dysfunctional and/or diseased and such organs can be treated by using a compound or composition of the present disclosure.

As used herein, the terms “amino acid,” refer to organic compounds with a carboxyl group at one end and a primary amino group at the other end. In a peptide, a carboxyl and an amino group form an amide bond, also called peptide bond, and any two or more amino acids linked together by a peptide bond is called a peptide. The term “residue” refers to a portion of a peptide derived from an amino acid. Amino acids with the amino group alpha to the carboxyl group are referred to as α-amino acids. These α-amino acids, as well as other types of amino acid, typically have another substituent at the alpha position known as the “side chain” or “R-group.” The chemical properties of the R-group greatly influence the broader chemical properties of the amino acid. In the present invention “cationic amino acids” are those with a cation on their side chain, preferably due to a protonated or alkylated amine group. An amino acid may be natural or proteinogenic, meaning that it occurs in nature and is used in the synthesis of polypeptides or proteins. An amino acid useful in the present disclosure can also be an unnatural or non-naturally occurring amino acid. For the purpose of the present disclosure, non-naturally occurring amino acids that can be used to build peptides are any organic compounds of less than 500 Da with a carboxyl group at one end and a primary amino group at the other end. This bi-functional molecule can be used to form amide bonds on either end. An amino end can condense with a carboxyl group of another molecule to form an amide bond and vice versa to form a polymer chain. Examples of unnatural amino acids in the present disclosure include 2-[2-(2-(aminoethoxy)ethoxy]acetic acid (abbreviated as Aeea), Diaminobutyric acid (abbreviated as Dab), and Diaminopropionic acid (Dap). For the purpose of describing the compounds of Formula (II) of the present disclosure, “cationic alkyl moiety” shall refer to the portion of Formula (II) that corresponds to Formula (I).

One-letter codes for naturally occurring amino acids are used herein. For example, alanine is A, arginine is R, asparagine is N, aspartic acid is D, cysteine is C, glutamic acid is E, glutamine is Q, glycine is G, histidine is H, isoleucine is I, leucine is L, lysine is K, methionine is M, phenylalanine is F, proline is P, serine is S, threonine is T, tryptophan is W, tyrosine is Y, valine is V, and Ornithine is O. For the purpose of the present disclosure, the one letter codes for amino acids may represent either stereoisomer of the amino acid, i.e. the L- or D-amino acid. For the purpose of the present disclosure, γE is glutamic acid where the sidechain carboxyl (gamma, γ) is the moiety used to link to the N-terminal portion of the peptide instead of the typical alpha carboxyl.

As used herein, the term “derivative” refers to a modified peptide resulting from any one or combination of 1) covalent addition of amino acid(s), moiety(ies), or a peptide with different inherent biological activity from the peptide being modified, 2) truncation or removal of one or more of amino acid in peptide backbone sequence, and/or 3) replacement of one or more of amino acid in the peptide backbone sequence, wherein the derivative maintains the inherent biological activity of the original peptide.

As used herein, the term “biological activity” means an activity of a peptide that is a measurable and inherent characteristic of the peptide. The biological activity of a native peptide is the affect that results after it binds to its receptor, prior to any modification or alteration in structure. In other words, it is the intrinsic biological activity of a peptide that can be measured after being exposed to its receptor in vitro or in vivo. When a modified peptide has measurable biological activity similar to the biological activity of the respective native or unmodified peptide, it can be considered that the modification did not eliminate the biological activity of the peptide. In other words, it can be considered that the modified peptide maintains the biological activity that is characteristic, inherent, or intrinsic of the native peptide.

As used herein, the term “bioavailability” refers to the proportion of a drug or other substance which enters blood circulation when introduced into the body and is able to have an active effect. Increased bioavailability can be measured using an assay that measures the blood level of a drug at corresponding time points after administration. One way of measuring bioavailability is to determine the area under the curve in a plot of blood level of a drug over time. In the present specification, a derivative of a drug that has much higher blood level compared to the parent drug (underivatized drug) at a corresponding timepoint after administration has higher bioavailability.

As used herein, “lethargy” means a clinical sign observed as inability or unwillingness to resume normal activity characterized by exploring and alertness. In the present disclosure, lethargic rats are easily observed by displaying little to no activity and/or unwillingness to explore around, half closed eyes, and hunched posture.

As used herein, “swelling” means a clinical sign observed as the enlargement or engorgement of body parts with blood or fluid associated with visible redness. It is caused by a buildup of fluid in the tissues. Swelling can occur all over the body (generalized) or only in one part of the body (localized). In the present disclosure swelling in rats induced by cationic alkyl-containing molecule or peptide is easily observed in the paws and snout and is associated with skin redness.

As used herein, “practical or reasonable mass dose” refers to a therapeutic dose that is practical to administer after allometric adjustment to a human dose, particularly in terms of the volume of the injectate that must remain in liquid form for parenteral bolus administration. A reasonable injectate volume in humans for non-intravenous parenteral bolus administration is 2 mL or less. A volume of greater than 2 mL is, although possible, considered not practical for non-intravenous parenteral administration in humans. An injectate volume of greater than 2 mL can be the result of the limited solubility of a peptide at the therapeutically effective dose. Therefore, considering the practical limitation of volume and solubility, one must reach the therapeutic dose within this volume parameter, outside of which is considered impractical. The dose can be evaluated across species using allometric scaling.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some aspects, a subject is a human.

As used herein, the phrase “therapeutically effective dose” refers to a bolus dose of a therapeutic agent (i.e., drug or therapeutic agent compound in μmol/kg or mg/kg) that elicits the biological or medicinal response that is being sought in a tissue, system, animal, individual or human by a researcher, veterinarian, medical doctor, or other clinician, which includes one or more of the following:
(1) changing the level of an analyte produced by the tissue and/or blood as a marker of biological response that can mitigate disease progression;
(2) preventing a disease, condition or disorder in an individual who may be predisposed to the disease, condition or disorder but does not yet experience or display the pathology or symptomatology of the disease;
(3) inhibiting a disease, condition or disorder in an individual who is experiencing or displaying the pathology or symptomatology of the disease, condition, or disorder; and
(4) ameliorating a disease, condition or disorder in an individual who is experiencing or displaying the pathology or symptomatology of the disease, condition, or disorder (i.e., reversing the pathology and/or symptomatology), such as decreasing the severity of a disease, prolonging survival time, and/or preventing death.

As used herein, the term “composition” refers to a substance, particularly a therapeutic substance, formulated, mixed, suspended, admixed, solvated, and/or co-crystalized with an excipient, carrier, or solvent. The present disclosure relates to compositions comprising Formula (I) or Formula (II) formulated with one or more pharmaceutically acceptable excipients. The compositions described herein can be used in accordance with the uses and/or methods described herein, e.g., to supply a compound of Formula (I) or Formula (II) for administration to a subject. Compositions include, for example, pharmaceutical, or medical, compositions.

As used herein, the terms “peptide” and “polypeptide” refer to polymers of amino acids. “Peptide” refers to a polypeptide with three or more amino acids covalently linked together by amide bonds through alpha amino and alpha carboxyl groups. The number of amino acid residues in a peptide can range from 3 to about 100 residues. The amino acid residues in a polypeptide or peptide can be canonical or non-canonical, as well as modified or unmodified. As used herein, the term “protein” refers to a polypeptide large enough to have a 3-dimensional structure, such as a β-barrel, or an α-helix. Examples of peptides that are suitable for inclusion in the compounds and conjugated peptides of the disclosure include, for example, the peptides of any od SEQ ID Nos: 23-50.

As used herein, the terms “subcutaneous administration”, “administered subcutaneously”, “s.c.”, “s.c. administration,” “SC,” and “SC administration” refer to delivery of a drug, usually in liquid form, directly into the fatty tissues just beneath the skin. The delivery is usually carried out by direct injection. These injections are shallower than those injected into muscle tissues. Providers often use subcutaneous injections for medications that are suitable for absorption into the bloodstream slowly and steadily.

As used herein, the terms “intravenous administration,” “IV administration,” and “IV injection” refer to delivery of drug, typically in liquid form, directly into a vein of an animal or human. The delivery method is usually by direct injection. The intravenous route of administration can be used both for injections, using a syringe at higher pressures; as well as for infusions, for example, using the pressure supplied by gravity.

As used herein, the terms “intramuscular administration,” “IM administration,” and “IM injection” refer to delivery of a drug, usually in liquid form, directly into the muscles of an animal or human. The delivery methods are usually by direct injection. This allows the medication to be absorbed into the bloodstream quickly. In some instances, a person may self-administer an IM injection. In some embodiments, IM injections can be used instead of intravenous injections, for example, when certain therapeutic agents are irritating to veins, or when a suitable vein can't be located.

As used herein, the term “nasal administration” refers to delivery of a therapeutic agent (e.g., in form of gel, liquid, aerosol, gas, or powder) by topical application, dropping as a liquid or insufflation into the nose of an animal or a human, for example by blowing or spraying. This form of administration can be used, depending on the formulation, to deliver a therapeutic agent to the nasal cavity or the lungs (depending on the device used). The therapeutic agent may not be absorbed systemically (purely local administration), may be totally absorbed systemically (purely systemic), or partially absorbed both locally and systemically. Nasal sprays can include locally acting drugs whose systemic effects are typically minimal.

As used herein, the terms “inhalation administration” and “administration by inhalation” refer to delivery of a therapeutic agent-often in the form of a gas or aerosol-via the mouth or the nose (insufflation). Inhalation administration enables the therapeutic agent to reach the body tissues rapidly by permitting almost instant contact with the blood supplying the alveoli (air sacs) of the lungs. In experimental animals, inhalation is analogous to intratracheal administration (IT) which avoids the nasal region and thus distinguishes inhalation from nasal administration (intranasal). In this specification intratracheal administration (IT) is analogous to administration by inhalation.

As used herein, the terms “parenteral” and “non-gastrointestinal” administration refer to a route of administration that is not through enteral or gastrointestinal routes. Examples of parenteral administration include subcutaneous (under the skin), intravenous (into a vein), intra-arterial (into an artery), intramuscular (into a muscle), intraperitoneal (infusion or injection into the peritoneum), inhalation (e.g., by intratracheal or inhalation administration into the lower respiratory tract), nasal administration (through the nose), sublingual and buccal medication, intrathecal (into the spinal canal), intracerebral (into the cerebrum), intracerebroventricular (into the cerebral ventricles), intradermal (into the skin itself), or any other administration route not involving the gastrointestinal tract. As used herein, the term “enteral” means administration to any region of the alimentary tract including the mouth (oral), pharynx (throat), esophagus, stomach, small intestine, large intestine, rectum, anus and any artificial opening in any of these regions.

As used herein, the term “excipient” refers to a substance that is formulated together with or mixed with an active pharmaceutical ingredient for the purpose of providing long-term stabilization, bulking up formulations that contain potent active ingredients in small amounts (thus often referred to as “bulking agents”, “fillers”, or “diluents”), and/or conferring a therapeutic enhancement on the active pharmaceutical ingredient in the final dosage form, such as to improve drug absorption, potency, dose, viscosity, solubility, and/or duration of action or presence of the active pharmaceutical ingredient in the blood. The selection of appropriate excipients depends upon the route of administration, the dosage form, the active pharmaceutical ingredient, and other factors. The excipient can include, a sugar, amino acid, buffer, antioxidant, chelating agent, solvent, vehicle, and/or complex polymer that binds and stabilizes an active pharmaceutical ingredient in vitro and/or in vivo. Though excipients were at one time assumed to be “inactive” ingredients, it is now understood that they can sometimes be a key determinant of dosage form performance. In other words, the effects of an excipient on pharmacodynamics and pharmacokinetics can be important and can require extensive research and study. Indeed, how an excipient influences delivery of an active pharmaceutical ingredient is often unpredictable.

For the purposes of the present disclosure, “ataxia” will mean “toxicity,” and is characterized as a clinical sign observed as poor muscle control that causes clumsy voluntary movements and/or death. It is observed as difficulty with mobility, coordination, and eye movements. In the determination of toxicity (where observation of ataxia is used as a marker of toxicity), the bolus doses were escalated until ataxia (i.e., toxicity) was seen, up to 10 μmol/kg, the maximum practical bolus dose of a peptide administered parenterally in a small volume of injectate. As used in the present disclosure, “ataxia” is also a clinical sign observed as poor muscle control and coordination, leading to awkward, unwieldy, or clumsy voluntary movements. Presented in the examples are rats with ataxia that showed difficulty with mobility, coordination, and/or eye movements. Doses that cause ataxia and/or death are considered toxic doses. As used herein, finding the “maximum tolerated dose” or “MTD” refers to the in vivo safety evaluation of a compound under investigation. For the present disclosure, the MTD of a test compound is the highest bolus dose showing no observed adverse effect or ataxia. Lethargy is reversible and not considered adverse. In other terms, the MTD is the highest bolus dose that caused no visible or observable toxicity after administration to a group of animals when compared to a control group. The control group is a vehicle group, in the case of testing the MTD of a cationic alkyl moiety or peptide-cationic alkyl conjugate. In the present disclosure, the effects commonly observed following cationic alkyl moiety administration included mainly reversible swelling, changes in coloration of the skin, and reversible lethargy.

As used herein, the terms “therapeutic index” “TI” and “therapeutic ratio” are a quantitative measure of the relative safety of a drug. For the purpose of the present disclosure, the TI is the ratio of the highest bolus dose that does not cause adverse effect such as ataxia or lethargy to the highest bolus dose that does not cause observable peripheral discoloration. The highest bolus does that does not cause adverse effect relative to a control may also be called the “no observed adverse effect level” or “NOAEL.” The highest bolus dose that does not cause observable peripheral discoloration compared to untreated control may also be called the “no observed effect level” or “NOEL.” Thus, the TI can also be interpreted as NOAEL/NOEL. A peptide with a high therapeutic index will have a better safety profile than one with a low therapeutic index, by providing a wider margin of safety for dosing during the treatment of the disease. This NOAEL/NOEL ratio, being a quantitative measure of the relative safety, is a comparison of the bolus dose that causes the beginning of therapeutic effect (e.g. vasodilation for the purposes of the examples in the present specification) to the highest bolus dose before the dose that causes toxicity.

For the purposes of present specification, peripheral discoloration includes redness of the extremities (e.g., skin of the hands, feet, ears, and/or lips). In rats, peripheral vasodilation is associated with redness that may or may not include swelling, while peripheral vasoconstriction is associated with paleness of the extremities. Redness or paleness are determined by a side-by-side comparison of treated subjects with untreated control subjects. The Sprague Dawley rats used in the included exemplification are white rats with easily visualized changes in coloration.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the FIGURES and/or TABLES should not be viewed as limiting. It should be understood that other embodiments may include more or less of each element shown in each FIGURE and/or TABLE. Further, some of the illustrated elements may be combined or omitted. Yet further, an example embodiment may include elements that are not illustrated in the FIGURES and/or TABLES. As used herein, with respect to measurements, “about” means +/−5%. As used herein, a recited range includes the end points, such that from 0.5 mole percent to 99.5 mole percent includes both 0.5 mole percent and 99.5 mole percent.

### Non-Limiting Embodiments of the Disclosure

Embodiment 1. A compound comprising a cationic alkyl moiety of Formula (I):

J-(CH2)x(CO)-(A)y-(B)z- (I),
wherein:
J is either HOOC or CH3;
x is 10-16;
A is independently selected from the group consisting of: 2-[2-(2-Aminoethoxy)ethoxy]acetic Acid (Aeea), gamma amino butyric acid (γAbu), gamma linked glutamate (γE), and alpha linked glutamate (E);
y is 2-4;
B is independently Diamino propionic acid (Dap) or Diamino butanoic acid (Dab); and
z is 2-4;
wherein -(B)z- comprises no more than 2 Dab residues and wherein the Dap or Dab residues are linked through the alpha amino.

Embodiment 2. The compound of Embodiment 1, wherein:
J is CH3;
x is 10, 12, 14, or 16;
A is independently selected from the group consisting of: 2-[2-(2-Aminoethoxy)ethoxy]acetic Acid (Aeea), and gamma amino butyric acid (γAbu), y is 3;
B is independently Diamino propionic acid (Dap) or Diamino butanoic acid (Dab); and
z is 2 or 3.

Embodiment 3. The compound of Embodiment 1, wherein:
J is CH3;
x is 10, 12, 14, or 16;
A is independently selected from the group consisting of: 2-[2-(2-Aminoethoxy)ethoxy]acetic Acid (Aeea), and gamma linked glutamate (YE);
y is 3;
B is independently Diamino propionic acid (Dap) or Diamino butanoic acid (Dab); and z is 2 or 3.

Embodiment 4. The compound of Embodiment 1, wherein:
J is CH3;
x is 10, 12, 14, or 16;
A is 2-[2-(2-Aminoethoxy)ethoxy]acetic Acid (Aeea),
y is 3;
B is independently Diamino propionic acid (Dap) or Diamino butanoic acid (Dab); and
z is 2 or 3.

Embodiment 5. The compound of Embodiment 1, wherein:
J is CH3;
x is 10, 12, 14, or 16;
A is gamma amino butyric acid (γAbu),
y is 3;
B is independently Diamino propionic acid (Dap) or Diamino butanoic acid (Dab); and
z is 2 or 3.

Embodiment 6. The compound of Embodiment 1, wherein:
J is HOOC;
x is 10, 12, 14, or 16;
A is independently selected from the group consisting of: 2-[2-(2-Aminoethoxy)ethoxy]acetic Acid (Aeea), and gamma amino butyric acid (γAbu),
y is 3;
B is independently Diamino propionic acid (Dap) or Diamino butanoic acid (Dab); and
z is 2 or 3.

Embodiment 7. The compound of Embodiment 1, wherein:
J is HOOC;
x is 10, 12, 14, or 16;
A is independently selected from the group consisting of: 2-[2-(2-Aminoethoxy)ethoxy]acetic Acid (Aeea), and gamma linked glutamate (7E),
y is 3;
B is independently Diamino propionic acid (Dap) or Diamino butanoic acid (Dab); and
z is 2 or 3.

Embodiment 8. The compound of Embodiment 1, wherein:
J is HOOC;
x is 10, 12, 14, or 16;
A is 2-[2-(2-Aminoethoxy)ethoxy]acetic Acid (Aeea),
y is 3;
B is independently Diamino propionic acid (Dap) or Diamino butanoic acid (Dab); and
z is 2 or 3.

Embodiment 9. The compound of Embodiment 1, wherein:
J is HOOC;
x is 10, 12, 14, or 16;
A is gamma linked glutamate (γE),
y is 3;
B is independently Diamino propionic acid (Dap) or Diamino butanoic acid (Dab); and
z is 2 or 3.

Embodiment 10. The compound of Embodiment 1, wherein:
J is either CH3;
x is 14;
(A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and
(B)z is Dap-Dap, Dab-Dab, Dab-Dap, or Dap-Dab.

Embodiment 11. The compound of Embodiment 1, wherein:
J is either CH3;
x is 14;
(A)y is Aeea-Aeea-Aeea, γAbu-γAbu-γAbu, γAbu-Aeea-Aeea, γE-Aeea-Aeea, or E-Aeea-Aeea; and
(B)z is Dap-Dap, Dab-Dab, Dab-Dap, or Dap-Dab.

Embodiment 12. The compound of Embodiment 1, wherein the cationic alkyl moiety is selected from SEQ ID NOs: 10 to 22 and 51 to 69.

Embodiment 13. The compound of Embodiment 1, wherein the cationic alkyl moiety is selected from SEQ ID NOs: 10 to 22.

Embodiment 14. The compound of any one of Embodiments 1-13, wherein the compound, when conjugated to a peptide, has no clinically observable ataxia after parenteral bolus administration in rats at a dose of 10 μmol/kg and lower.

Embodiment 15. The compound of any one of Embodiments 1-14, for use in modifying a peptide via covalent conjugation.

Embodiment 16. A conjugated peptide of Formula (II):

CH3(CH2)x(CO)-(A)y-(B)z-Peptide (II)
wherein:
   x is 10-16;
   A is independently selected from the group consisting of: 2-[2-(2-Aminoethoxy)ethoxy]acetic Acid (Aeea), gamma amino butyric acid (γAbu), gamma linked glutamate (γE), and alpha linked glutamate (E);
   y is 2-4;
   B is independently Diamino propionic acid (Dap) or Diamino butanoic acid (Dab); and
   z is 2-4;
wherein:
   (B)z- comprises no more than 2 Dab residues and wherein the Dap or Dab residues are linked through the alpha amino;
   CH3(CH2)x(CO)-(A)y-(B)z- is covalently linked to the N-terminus of Peptide or linked to one of the side chain amino groups of Peptide;
   optionally, wherein the conjugated peptide has biological activity that is equivalent or higher than the unmodified peptide at an equivalent bolus dose; and/or
   the conjugated peptide has an equivalent or higher blood level than the unconjugated peptide at the same time-point after a bolus administration at an equivalent dose.

Embodiment 17. The conjugated peptide of Embodiment 16, wherein the conjugated peptide binds to a natriuretic peptide receptor and has no adverse effect or ataxia at a bolus dose of 3.0 μmol/kg and lower in rats.

Embodiment 18. The conjugated peptide of Embodiment 16 or 17, wherein the CH3(CH2)x(CO)-(A)y-(B)z- moiety is covalently linked to the N-terminus of Peptide.

Embodiment 19. The conjugated peptide of any one of Embodiments 16-18, wherein Peptide is a natriuretic peptide of SEQ ID NO: 32, 44, 48, or 75, or a natriuretic peptide derivative.

Embodiment 20. The conjugated peptide of any one of Embodiments 16-19, wherein the CH3(CH2)x(CO)-(A)y-(B)z- moiety is selected from SEQ ID NOs: 10 to 22 and 51 to 69.

Embodiment 21. The conjugated peptide of Embodiment 20, wherein the CH3(CH2)x(CO)-(A)y-(B)z- moiety is selected from SEQ ID NOs: 10 to 22.

Embodiment 22. The conjugated peptide of any one of Embodiments 16-20, wherein Peptide is a natriuretic peptide derivative with one or more methionine residues replaced by glutamine (Q), Leucine (L), Norleucine (Nle), or methoxinine (Mox).

Embodiment 23. The conjugated peptide of any one of Embodiments 16-22, wherein Peptide is a natriuretic peptide according to SEQ ID NO: 32, or a derivative thereof wherein one or more methionine residues are replaced by glutamine (Q), and the CH3(CH2)x(CO)-(A)y-(B)z-moiety is selected from SEQ ID NOs: 10 to 22.

Embodiment 24. The conjugated peptide of any one of Embodiments 16-23, wherein the conjugated peptide is selected from SEQ ID NOs: 29-31, 33-43, 45-47, 49-51.

Embodiment 25. The conjugated peptide of Embodiment 24, wherein the conjugated peptide is selected from SEQ ID NOs: 29-31 and 33-43.

Embodiment 26. The conjugated peptide of Embodiment 25, wherein the conjugated peptide is selected from SEQ ID NOs: 29-31.

Embodiment 27. The conjugated peptide of Embodiment 26, wherein the conjugated peptide is SEQ ID NO: 29.

Embodiment 28. The conjugated peptide of Embodiment 26, wherein the conjugated peptide is SEQ ID NO: 30.

Embodiment 29. The conjugated peptide of Embodiment 26, wherein the conjugated peptide of Formula (II) is SEQ ID NO: 31.

Embodiment 30. The conjugated peptide of any one of Embodiments 16-29, wherein the conjugated peptide binds to natriuretic peptide receptor B (NPRB), natriuretic peptide receptor C (NPRC), or a combination thereof.

Embodiment 31. The conjugated peptide of any one of Embodiments 16-30, wherein the conjugated peptide is a NPRB agonist.

Embodiment 32. The conjugated peptide of any one of Embodiments 16-31, wherein the conjugated peptide is a NPRC agonist.

Embodiment 33. The conjugated peptide of any one of Embodiments 16-32, wherein the conjugated peptide generates a physiological effect selected from: prolonged increase blood cGMP, changes in cAMP, changes in blood pressure, increased survival from Sepsis, increased survival from Acute Lung Injury, increase survival from Acute Respiratory Distress Syndrome, decrease in MPO positive cells, decrease in number of cells in Alveolar Fluid or in Bronchoalveolar Lavage Fluid, decrease in amount of protein in Alveolar Fluid or in Bronchoalveolar Lavage fluid, decrease endothelial permeability, decrease in lung weight per body weight, decrease in Monocyte Chemoattractant Protein-1, decrease in IL-6, decrease TNF-alpha, decrease in A1008/A9, decrease fibrosis, decrease in tumor volume, decrease metastasis, decrease inflammation, antiproliferative effects, decrease cancer burden, inhibition of cyclooxygenase 2 (COX-2) expression, antagonizing the renin-angiotensin-aldosterone system, inhibiting cardiac hypertrophy, or a combination thereof.

Embodiment 34. The compound of any one of Embodiments 1-15 or the conjugated peptide of any one of Embodiments 16-33 for use in the manufacture of a medical composition.

Embodiment 35. The compound or conjugated peptide for use of Embodiment 34, wherein the medical composition comprises one or more pharmaceutically acceptable carriers or excipients.

Embodiment 36. The compound or conjugated peptide for use of Embodiment 35, wherein the one or more pharmaceutically acceptable carriers or excipients comprises a bulking agent, a buffering agent, a stabilizer, a preservative, or a combination thereof.

Embodiment 37. The compound of any one of Embodiments 1-15 or the conjugated peptide of any one of Embodiments 16-33, for use in treating a disease or condition in a subject in need thereof.

Embodiment 38. The compound or conjugated peptide for use of Embodiment 37, wherein the compound or conjugated peptide is selected from:
a) any one of SEQ ID NOS: 29-31, 33-43, 45-47, 49-51, or
b) any one of SEQ ID NOS: 29-31, 33-43, 45-47, or
c) any one of SEQ ID NOS: 29-31, 33-43, or
d) any one of SEQ ID NOS: 29-31, or
e) SEQ ID NO: 29 or
f) SEQ ID NO: 30, or
g) SEQ ID NO: 31.

Embodiment 39. The compound for use in anyone of Embodiment 37 or 38, wherein the compound or conjugated peptide comprises SEQ ID NO: 31.

Embodiment 40. The compound or conjugated peptide for use of any one of Embodiments 37-39, wherein the disease or condition affects the lung (e.g., ALI, ARDS, COVID, inflammation, sepsis, fibrosis, or cancer), liver (e.g., non-alcoholic fatty liver disease (NAFLD), non-alcoholic steatohepatitis (NASH), inflammation, fibrosis, or cancer), heart (e.g., heart failure with preserved ejection fraction (HFpEF), heart failure with reduced ejection fraction (HFrEF), acute heart failure, or congestive heart failure), bone/joint (e.g., osteoporosis, osteoarthritis, rheumatoid arthritis, inflammation, cancer, or dwarfism), kidney (e.g., chronic kidney disease (CKD), acute kidney injury (AKI), drug induced kidney injury, inflammation/nephritis, kidney fibrosis, glomerulosclerosis, or kidney cancer), prostate (e.g., prostate hyperplasia or prostate cancer), brain, eye, skin, muscle, blood, gastrointestinal track, bladder, testis, ovary, uterus, and/or blood vessel.

Embodiment 41. The compound or conjugated peptide for use of any one of Embodiments 37-39, wherein the disease or condition is pre-metastatic or post-metastatic cancer.

Embodiment 42. The compound or conjugated peptide for use of Embodiment 41, wherein the cancer is a cancer of any one or more organs selected from lung, lung pleura, liver, heart, bone/joint, kidney, prostate, breast, brain, eye, skin, muscle, blood, blood vessels, gastrointestinal track, bladder, testis, ovary, and/or uterus.

Embodiment 43. The compound or conjugated peptide for use of any one of Embodiments 37-39, wherein the disease or condition is pneumonia, acute lung injury (ALI), acute respiratory distress syndrome (ARDS), or COVID in a subject in need thereof.

Embodiment 44. The compound or conjugated peptide for use of any one of Embodiments 37-39, wherein the disease or condition is fibrosis.

Embodiment 45. The compound or conjugated peptide for use of any one of Embodiments 37-44, wherein the treating comprises administering to the subject a therapeutically effective bolus dose of 10.0 μmol/kg or lower, and/or between 10.0 μmol/kg and 0.0001 μmol/kg inclusive.

Embodiment 46. The compound or conjugated peptide for use of any one of Embodiments 37-45, wherein the compound is administered to the subject either as a monotherapy or in combination with one or more additional agents or treatments.

Embodiment 47. The compound or conjugated peptide for use of Embodiment 46, wherein when the one or more additional agents or treatments are selected from immune check point inhibitors, surgery/amputation, radiation, chemotherapy, or a combination thereof.

Embodiment 48. The compound for use of any one of Embodiments 37-46, wherein the compound is administered subcutaneously, by infusion, by inhalation, by nasal spray, orally, eye drops, and/or by topical application.

Embodiment 49. The compound or conjugated peptide for use of any one of Embodiments 37-42, wherein the compound or conjugated peptide is administered to the subject subcutaneously, by infusion, by inhalation, by nasal spray, orally, eye drops, and/or by topical application.

Embodiment 50. A composition comprising the compound of any one of Embodiments 1-15 or the conjugated peptide of any one of Embodiments 16-33, and one or more pharmaceutically acceptable carriers or excipients.

Embodiment 51. The composition of Embodiment 49, wherein the one or more pharmaceutically acceptable carriers or excipients comprises a bulking agent, a buffering agent, a stabilizer, a preservative, or a combination thereof.

### EXAMPLES

Example 1: Clinically observable adverse effects of various cationic alkyl moieties tested at 3.0, 5.0, or 10 μmol/kg showed that cationic alkyl moieties containing Dap and/or Dab as a cationic residue are much less toxic than those containing cationic amino acids with longer R-group.

The following experimental protocol was followed to generate data for Table 1. Animal care and treatment: Male Sprague Dawley rats (8 to 10 weeks old, Charles River, Hollister, CA) were housed in pairs (n=3/group) in disposable polypropylene cages with rodent cob bedding in the PharmaIN animal facility. The animals had ad libitum access to food (LabDiet Pico Rodent #5053; Animal Specialties, Woodburn, OR) and water. The temperature (68-74° F.) and humidity (30-60%) were kept within a controlled range, and a 12-hour light/dark cycle was used. Drug administration and observation: Each rat received a subcutaneous injection of a cationic alkyl modifier (SEQ ID NO: 1-22; listed in Table 1 below) in a buffer of sterile water adjusted to a pH between 4.5-5, with an initial bolus dose of 5.0 μmol/kg and an injection volume of 1.0 mL/kg. The bolus dose level was adjusted based on observations, and animals were monitored closely for lethargy, ataxia, swelling, or discoloration during the first 4 hours post-injection and hourly thereafter. As used herein, “ataxia” means a clinical sign observed as poor muscle control that causes clumsy voluntary movements. It may cause difficulty with mobility, coordination, and eye movements. As used herein, “lethargy” means a clinical sign observed as inability or unwillingness to resume normal activity characterized by exploring and alertness. Lethargic rats are easily observed by displaying little to no activity and/or unwillingness to explore around, half closed eyes, and hunched posture. As used herein, “swelling” means a clinical sign observed as the enlargement or engorgement of body parts with blood or fluid and is associated with visible redness (discoloration). If no swelling occurred, dose level was increased to 10 μmol/kg; if swelling was present, bolus dose level was decreased to 3.0 μmol/kg. A wash-out period of 1 week was allowed between bolus doses, and animals were humanely euthanized through CO₂ asphyxiation after final observation.

TABLE 1
Based on clinically observable adverse effects of various
cationic alkyl sequences tested at 3.0, 5.0, or 10 μmol/kg,
those cationic alkyl moieties with longer R-group
cationic amino acids (both natural and unnatural)
have higher toxicity than those with Dap and/or Dab.

| | | Swelling | | |
|---|---|---|---|---|
| | | and/or | | |
| | | Discoloration | Lethargy* | Ataxia** |
| | | in n = 3 | in n = 3 | in n = 3 |
| | SEQ ID | Unit: | Unit: | Unit: |
| Sequence | NO: | μmol/kg | μmol/kg | μmol/kg |
| CH3(CH2)₁₄(C═O)- | SEQ ID | ≥3 | ≥3 | ≥3 |
| Aeea-Aeea-Aeea-kK | NO: 1 | | | |
| CH3(CH2)₁₆(C═O)- | SEQ ID | ≥3 | ≥3 | >3, ≥5 |
| KKKKGGG | NO: 2 | | | |
| CH3(CH2)₁₄(C═O)- | SEQ ID | ≥3 | ≥5 | ≥5 |
| Aeea-Aeea-Aeea-kk | NO: 3 | | | |
| CH3(CH2)₁₄(C═O)- | SEQ ID | ≥3 | ≥3 | ≥3 |
| Aeea-Aeea-KKKK | NO: 4 | | | |
| CH3(CH2)₁₄(C═O)- | SEQ ID | >3, ≥5 | ≥5 | ≥5 |
| Aeea-Aeea-KK | NO: 5 | | | |
| CH3(CH2)₁₄(C═O)- | SEQ ID | >3, ≥5 | >3, ≥5 | >3, ≥5 |
| Aeea-Aeea-R-R | NO: 6 | | | |
| CH3(CH2)₁₄(C═O)- | SEQ ID | ≥3 | ≥5 | >5 |
| Aeea-Aeea-Aeea-KK | NO: 7 | | | |
| CH3(CH2)₁₄(C═O)- | SEQ ID | ≥3 | ≥3 | >5 |
| Aeea-Aeea-Aeea-Dab- | NO: 8 | | | |
| Dab-Dab-Dab | | | | |
| CH3(CH2)₁₄(C═O)- | SEQ ID | ≥3 | >5 | >5 |
| Aeea-Aeea-Aeea-Dab- | NO: 9 | | | |
| Dab-Dab | | | | |
| CH3(CH2)₁₄(C═O)- | SEQ ID | ≥10 | >10 | >10 |
| Aeea-Aeea-Aeea-Dab- | NO: 10 | | | |
| Dab | | | | |
| CH3(CH2)₁₄(C═O)- | SEQ ID | >10 | >10 | >10 |
| γAbu-γAbu-Dab-Dab | NO: 11 | | | |
| CH3(CH2)₁₄(C═O)- | SEQ ID | >10 | >10 | >10 |
| γAbu-γAbu-γAbu-Dab- | NO: 12 | | | |
| Dab | | | | |
| CH3(CH2)₁₄(C═O)- | SEQ ID | >10 | >10 | >10 |
| Aeea-Aeea-Dap-Dab | NO: 13 | | | |
| CH3(CH2)₁₄(C═O)- | SEQ ID | >10 | >10 | >10 |
| Aeea-Aeea-Aeea-Dap- | NO: 14 | | | |
| Dap | | | | |
| CH3(CH2)₁₄(C═O)-VE- | SEQ ID | >10 | >10 | >10 |
| Aeea-Aeea-Dab-Dab | NO: 15 | | | |
| CH3(CH2)₁₄(C═O)-E- | SEQ ID | >10 | >10 | >10 |
| Aeea-Aeea-Dab-Dab | NO: 16 | | | |
| CH3(CH2)₁₄(C═O)- | SEQ ID | >10 | >10 | >10 |
| Aeea-Aeea-Aeea-Dab- | NO: 17 | | | |
| Dap | | | | |
| (HOOC)(CH2)₁₆(C═O)- | SEQ ID | >10 | >10 | >10 |
| Aeea-Aeea-Aeea-Dab- | NO: 18 | | | |
| Dab | | | | |
| CH3(CH2)₁₄(C═O)- | SEQ ID | >10 | >10 | >10 |
| Aeea-Aeea-Aeea-Dap- | NO: 19 | | | |
| Dap-Dap | | | | |
| (HOOC)(CH2)₁₆(C═O)- | SEQ ID | >10 | >10 | >10 |
| Aeea-Aeea-Aeea-Dap- | NO: 20 | | | |
| Dap | | | | |
| (HOOC)(CH2)₁₆(C═O)- | SEQ ID | >10 | >10 | >10 |
| Aeea-Aeea-Dab-Dab | NO: 21 | | | |
| CH3(CH2)₁₄(C═O)- | SEQ ID | >10 | >10 | >10 |
| Aeea-Aeea-Aeea-Dap- | NO: 22 | | | |
| Dap-Dap-Dap | | | | |

*Lethargy is considered maximum tolerated bolus dose
**Ataxia is considered a toxic/adverse effect and is recognized as bolus dose limiting toxicity

Example 2: Alkylated Natriuretic Peptides containing alkylated Dap/Dab resulted in fewer clinically observable adverse effects or ataxia in rats compared to those containing longer R-group cationic amino acids such as lysine or arginine or their unnatural D-isomers. Moreover, the alkylated peptides exhibited improved pharmacokinetic and/or pharmacodynamic properties compared to the unmodified peptide (see Example 3, Table 3).

The following experimental protocol was followed to generate data for Table 2. Animal care and treatment: Male Sprague Dawley rats aged 8 to 10 weeks (Charles River, Hollister, CA) were housed in groups of two (n=3/group) in disposable polypropylene cages with rodent cob bedding at the PharmaIN animal facility. Animals had ad libitum access to food (LabDiet Pico Rodent #5053; Animal Specialties, Woodburn, OR) and water. Temperature (68-74° F.) and humidity (30-60%) were maintained within a controlled range, and a 12-hour light/dark cycle was used. Drug administration and observation: During these dose-range finding studies, rats were treated with up to 10 mg/kg of cationic alkyl modified CNP (SEQ ID NO: 23-31 listed in Table 2 below; PharmaIN, Bothell, WA) in lead buffer, via subcutaneous bolus administration between the shoulder blades at the dose volume of 1.0 mL/kg. Animals were monitored closely during the first 4 hours following injection, then hourly throughout the rest of the day for any signs of swelling or discoloration (dilatory side effect), lethargy (side effect), ataxia (ataxia is used as a marker of toxicity). Rats were allowed a 1-week wash-out period between bolus doses. Following final observations, animals were humanely euthanized through CO₂ asphyxiation. Cationic alkyl-containing longer R-group cationic amino acids (both natural and unnatural), such as lysine or arginine (e.g., CH3(CH2)14(C═O)-KKGGGKK-[SEQ ID NO: 70], CH3(CH2)14(C═O)-GGGKKKK-[SEQ ID NO: 71], CH3(CH2)14(C═O)-kkkkGGG-[SEQ ID NO: 72], CH3(CH2)14(C═O)-KKGGGRR-[SEQ ID NO: 73], CH3(CH2)14(C═O)-KKGGG-Dab-Dab-[SEQ ID NO: 74]) resulted in more toxic conjugates [e.g., SEQ ID NO: 23-28 (see Table 2)] compared to CNP modified with cationic alkyl moieties of Formula (I) [e.g., SEQ ID NO: 29-31 (see Table 2)].

TABLE 2
Compounds containing cationic alkyl moieties of Formula (I)
linked to CNP have much lower toxicity than CNP linked to
cationic alkyl moieties containing longer R-group cationic
amino acids (both natural and unnatural)
such as lysine or arginine.

| | | Swelling | | |
|---|---|---|---|---|
| | | and/or | | |
| | | Discoloration | Lethargy* | Ataxia** |
| | | in n = 3 | in n = 3 | in n = 3 |
| | | Unit: | Unit: | Unit: |
| | SEQ ID | mg/kg | mg/kg | mg/kg |
| Sequence | NO: | (μmol/kg) | (μmol/kg) | (μmol/kg) |
| CH3(CH2)₁₆(C═O)- | SEQ ID | ≥1 | ≥1 | ≥3 |
| KKKKGGG- | NO: 23 | (0.31) | (0.31) | (0.94) |
| GLSKGCFGLKLDRIG | | | | |
| SQSGLGC (disulfide | | | | |
| bond) | | | | |
| CH3(CH2)₁₄(C═O)- | SEQ ID | ≥5 | ≥5 | ≥5 |
| KKGGGKK- | NO: 24 | (1.6) | (1.6) | (1.6) |
| GLSKGCFGLKLDRIG | | | | |
| SQSGLGC (disulfide | | | | |
| bond) | | | | |
| CH3(CH2)₁₄(C═O)- | SEQ ID | ≥5 | ≥5 | ≥5 |
| GGGKKKK- | NO: 25 | (1.6) | (1.6) | (1.6) |
| GLSKGCFGLKLDRIG | | | | |
| SQSGLGC (disulfide | | | | |
| bond) | | | | |
| CH3(CH2)₁₄(C═O)- | SEQ ID | ≥5 | ≥5 | ≥5 |
| kkkkGGG- | NO: 26 | (1.6) | (1.6) | (1.6) |
| GLSKGCFGLKLDRIG | | | | |
| SQSGLGC (disulfide | | | | |
| bond) | | | | |
| CH3(CH2)₄(C═O)- | SEQ ID | ≥5 | ≥5 | ≥5 |
| KKGGGRR- | NO: 27 | (1.6) | (1.6) | (1.6) |
| GLSKGCFGLKLDRIG | | | | |
| SQSGLGC (disulfide | | | | |
| bond) | | | | |
| CH3(CH2)₄(C═O)- | SEQ ID | ≥5 | ≥5 | ≥5 |
| KKGGG-Dab-Dab- | NO: 28 | (1.6) | (1.6) | (1.6) |
| GLSKGCFGLKLDRIG | | | | |
| SQSGLGC (disulfide | | | | |
| bond) | | | | |
| CH3(CH2)₁₄(C═O)- | SEQ ID | ≥9.5 | ≥9.5 | >10 |
| Aeea-Aeea-Aeea- | NO: 29 | (3.1) | (3.1) | (3.3) |
| Dab-Dab- | | | | |
| GLSKGCFGLKLDRIG | | | | |
| SQSGLGC (disulfide | | | | |
| bond) | | | | |
| CH3(CH2)₁₄(C═O)- | SEQ ID | >10 | ≥10 | >10 |
| YE-Aeea-Aeea-Dab- | NO: 30 | (3.3) | (3.3) | (3.3) |
| Dab- | | | | |
| GLSKGCFGLKLDRIG | | | | |
| SQSGLGC (disulfide | | | | |
| bond) | | | | |
| CH3(CH2)₁₄(C═O)- | SEQ ID | >10 | >10 | >10 |
| Aeea-Aeea-Aeea- | NO: 31 | (3.3) | (3.3) | (3.3) |
| Dap-Dap- | | | | |
| GLSKGCFGLKLDRIG | | | | |
| SQSGLGC (disulfide | | | | |
| bond) | | | | |

*Lethargy is considered maximum tolerated bolus dose
**Ataxia is considered a toxic/adverse effect and is recognized as bolus dose limiting toxicity

Example 3: Pharmacokinetic and pharmacodynamic data for several Natriuretic Peptides (NP) modified with alkylated cationic Dap or Dab residues. These modifications exhibit improved pharmacodynamics, as evidenced by their higher 2- and 6-hour plasma cGMP levels. The pharmacokinetics are also improved as evidenced by their significant presence in the bloodstream (NP level), compared to the corresponding native NP when administered subcutaneously at a bolus dose of 1.0 mg/kg (≤0.45 μmol/kg) to mice.

The following experimental protocol was followed to generate data for Table 3. Animal care and treatment: Male CD-1 mice aged 6 to 9 weeks (Charles River, Hollister, CA) were housed in groups of 5-6 per disposable polypropylene cage with rodent cob bedding and given ad libitum access to LabDiet Pico Rodent #5053 food and water. The animals were kept in the PharmnaIN animal facility with controlled temperature (68-74° F.) and humidity (30-60%), and a 12-hour light/dark cycle. Drug administration and blood sampling: All animals were treated with one of the following natriuretic peptides: native human ANP (SEQ ID NO: 44; Chempep Inc., Wellington, FL), BNP (SEQ ID NO: 48; Tocris, Minneapolis, MN), CNP (SEQ ID NO: 32; Chempep Inc., Wellington, FL), or cationic alkyl modified NP (as listed in Table 3; PharmaIN, Bothell, WA) via subcutaneous injection between the shoulder blades at a bolus dose of 1.0 mg/kg (≤0.45 μmol/kg). The test articles were formulated or dissolved in sterile water for injection on the same day of administration. No adverse effects were observed in any of the animals at this bolus dose. Blood samples were collected via retro-orbital bleed at 2- and 6-hours post-injection, with two bleedings per animal at two different timepoints. The samples were collected in K₂EDTA tubes, processed by centrifugation (2000×g; 15 m 4 eC within 30 minutes after collection), and the resulting plasma samples were stored at −80° C. Biochemical analysis: The plasma concentration of the respective natriuretic peptide was analyzed using commercially available ELISA kits from Phoenix Pharmaceuticals (Burlingame, CA): ANP ELISA (cat #EKE-005-06), BNP ELISA (cat #EKE-011-03), and CNP ELISA (cat #EKE-012-03). The ANP and BNP kits detect only the respective derivatives, while the CNP kit detects the cyclic structure of CNP and all CNP derivatives with the same level of reactivity. Plasma cGMP was analyzed using a commercially available kit from Abcam (ab133052, Waltham, MA).

TABLE 3
Pharmacokinetic and pharmacodynamic data of several
Natriuretic Peptides (NP) modified with alkylated
cationic Dap or Dab residues compared to the corresponding
native NP when administered at a bolus dose of 1.0 mg/ml
(≤0.45 μmol/kg) to mice.

| | | Pharmacodynamics | | Pharmacokinetics | |
|---|---|---|---|---|---|
| | | Plasma | | Plasma | |
| | | concentration | | concentration | |
| | | of cGMP in | | of respective | |
| | | pmol/mL | | Natriuretic | |
| | | (SD; | | Peptide; | |
| | | n = 3-5). | | ANP, BNP, | |
| | | The Baseline | | or CNP | |
| | | blood cGMP | | in ng/ml | |
| | | level is 10 | | (SD; | |
| | SEQ ID | +/−8 pmol/ml | | n = 3-5) | |
| Sequence | NO: | 2 hr | 6 hr | 2 hr | 6 hr |
| Vehicle control | Blank | Refer to | Refer | 1.22 | 0.95 |
| - 300 mM | | table | to | (0.22) | (0.23) |
| Trehalose, 15 | | header | table | | |
| mM Succinate, | | | header | | |
| pH 4.5 | | | | | |
| GLSKGCFGLKLD | SEQ ID | 8.7 | 9.5 | 2.17 | 2.91 |
| RIGSMSGLGC | NO: 32 | (3.8) | (4.5) | (0.44) | (0.7) |
| CH3(CH2)₁₄(C═ | SEQ ID | 157.31* | 65.65* | 17.97* | 8.93* |
| O)-Aeea-Aeea- | NO: 29 | (47.48) | (34.63) | (12.65) | (4.09) |
| Aeea-Dab-Dab- | | | | | |
| GLSKGCFGLKLD | | | | | |
| RIGSQSGLGC | | | | | |
| (disulfide bond) | | | | | |
| (COOH)(CH2)₁₆ | SEQ ID | 25.91** | 31.61* | 75.93** | 68.17**** |
| (C═O)-Aeea- | NO: 33 | (5.67) | (11.92) | (43.72) | (12.31) |
| Aeea-Aeea- | | | | | |
| Dab-Dab- | | | | | |
| GLSKGCFGLKLD | | | | | |
| RIGSQSGLGC | | | | | |
| (disulfide bond) | | | | | |
| (COOH)(CH2)₁₆ | SEQ | 15.78* | 33.6 | 75.53**** | 41.05** |
| (C═O)-Aeea- | ID NO: | (3.08) | (32.25) | (13.88) | (24) |
| Aeea-Dab-Dab- | 34 | | | | |
| GLSKGCFGLKLD | | | | | |
| RIGSQSGLGC | | | | | |
| (disulfide | | | | | |
| bond) | | | | | |
| CH3(CH2)₁₄(C═ | SEQ ID | 217.30** | 83.85* | 12.58** | 1.90 |
| O)-vE-Aeea- | NO: 30 | (61.06) | ** | (5.57) | (0.49) |
| Aeea-Dab-Dab- | | | (21.13) | | |
| GLSKGCFGLKLD | | | | | |
| RIGSQSGLGC | | | | | |
| (disulfide bond) | | | | | |
| CH3(CH2)₁₄(C═ | SEQ | 98.34**** | 53.62*** | 76.83**** | 14.78*** |
| O)-Aeea-Aeea- | ID NO: | (22.73) | (17.26) | (13.35) | (3.78) |
| Aeea-Dap-Dap- | 31 | | | | |
| GLSKGCFGLKLD | | | | | |
| RIGSQSGLGC | | | | | |
| (disulfide bond) | | | | | |
| CH3(CH2)₁₄(C═ | SEQ ID | 65.28**** | 35.10** | 12.9**** | 4.13 (0.96) |
| O)-Aeea-Aeea- | NO: 35 | (9.27) | (7.45) | (0.89) | |
| Dap-Dab- | | | | | |
| GLSKGCFGLKLD | | | | | |
| RIGSQSGLGC | | | | | |
| (disulfide bond) | | | | | |
| CH3(CH2)₁₄(C═ | SEQ ID | 54.61*** | 41.75** | 7.57*** | 2.57 (0.90) |
| O)-yAbu-yAbu- | NO: 36 | (13.29) | (14.42) | (2.04) | |
| yAbu-Dab-Dab- | | | | | |
| GLSKGCFGLKLD | | | | | |
| RIGSQSGLGC | | | | | |
| (disulfide bond) | | | | | |
| CH3(CH2)₁₄(C═ | SEQ ID | 39.77* | 22.71** | 11.93** | 5.77*** |
| O)-yAbu-yAbu- | NO: 37 | (20.05) | (1.62) | (4.59) | (0.50) |
| Dab-Dab- | | | | | |
| GLSKGCFGLKLD | | | | | |
| RIGSQSGLGC | | | | | |
| (disulfide bond) | | | | | |
| CH3(CH2)₁₄(C═ | SEQ | 74.69* | 38.95*** | 31.70** | 25.86 |
| O)-E-Aeea- | ID NO: | (44.99) | (7.9) | (12.92) | (27.78) |
| Aeea-Dab-Dab- | 38 | | | | |
| GLSKGCFGLKLD | | | | | |
| RIGSQSGLGC | | | | | |
| (disulfide bond) | | | | | |
| (COOH)(CH2)₁₆ | SEQ ID | 13.29 | 43.60 | 50.69*** | 23.71 |
| (C═O)-Aeea- | NO: 39 | (8.83) | (38.19) | (13.6) | (21.56) |
| Aeea-Aeea- | | | | | |
| Dap-Dap- | | | | | |
| GLSKGCFGLKLD | | | | | |
| RIGSQSGLGC | | | | | |
| (disulfide bond) | | | | | |
| CH3(CH2)₁₄(C═ | SEQ | 120.72*** | 104.05 | 36.4** | 35.21* |
| O)-Aeea-Aeea- | ID NO: | (35.81) | | (14.97) | (25.2) |
| Aeea-Dap-Dap- | 40 | | | | |
| GLSKGCFGLKLD | | | | | |
| RIGSMSGLGC | | | | | |
| CH3(CH2)₁₄(C═ | SEQ ID | 124.77*** | 67.78**** | 26.73*** | 30.15*** |
| O)-Aeea-Aeea- | NO: 41 | (37.12) | (13.32) | (7.74) | (9.46) |
| Aeea-Dab- | | | | | |
| GLSKGCFGLKLD | | | | | |
| RIGSQSGLGC | | | | | |
| (disulfide bond) | | | | | |
| CH3(CH2)₁₄(C═ | SEQ | 110.21*** | 46.90** | 26.06**** | 25.0*** |
| O)-Aeea-Aeea- | ID NO: | (31.38) | (16.90) | (6.51) | (6.93) |
| Aeea-Dap- | 42 | | | | |
| GLSKGCFGLKLD | | | | | |
| RIGSQSGLGC | | | | | |
| (disulfide bond) | | | | | |
| CH3(CH2)₁₄(C═ | SEQ | 114.85**** | 93.39**** | 29.25*** | 16.83*** |
| O)-Aeea-Aeea- | ID NO: | (14.71) | (21.32) | (9.16) | (5.23) |
| Aeea-Dap-Dap- | 43 | | | | |
| Dap- | | | | | |
| GLSKGCFGLKLD | | | | | |
| RIGSQSGLGC | | | | | |
| (disulfide bond) | | | | | |
| Assay for ANP | Blank | Refer to | Refer | 30.14 | 27.32 |
| using | | table | to | (1.54) | (2.82) |
| blank/baseline | | header | table | | |
| | | | header | | |
| SLRRSSCFGGR | SEQ ID | 26.23 | 14.08 | 25.62 | 26.17 |
| MDRIGAQSGLG | NO: 44 | (12.19) | (8.57) | (13.85) | (9.51) |
| CNSFRY | | | | | |
| CH3(CH2)₁₄(C═ | SEQ ID | 123.82** | 16.18 | Not | Not |
| O)-Aeea-Aeea- | NO: 45 | (41.57) | (3.95) | recognized | recognized |
| Aeea-Dap-Dap- | | | | by ELISA. | by ELISA. |
| SLRRSSCFGGRL | | | | N.D. | N.D. |
| DRIGAQSGLGC | | | | | |
| NSFRY | | | | | |
| (disulfide bond) | | | | | |
| CH3(CH2)₁₄(C═ | SEQ | 76.68** | 17.11 | Not | Not |
| O)-Aeea-Aeea- | ID NO: | (15.65) | (0.73) | recognized | recognized |
| Aeea-Dap-Dap- | 46 | | | by ELISA. | by ELISA. |
| SLRRSSCFGGRQ | | | | N.D. | N.D. |
| DRIGAQSGLGC | | | | | |
| NSFRY | | | | | |
| (disulfide bond) | | | | | |
| CH3(CH2)₁₄(C═ | SEQ ID | 52.05 | 8.00 | 67.14** | 37.18 |
| O)-Aeea-Aeea- | NO: 47 | (18.27) | (1.16) | (15.78) | (22.59) |
| Aeea-Dap-Dap- | | | | | |
| SLRRSSCFGGR | | | | | |
| MDRIGAQSGLG | | | | | |
| CNSFRY | | | | | |
| (disulfide bond) | | | | | |
| Assay for BNP | Blank | Refer to | Refer | 32.93 | 30.65 |
| using | | table | to | (5.7) | (5.24) |
| blank/baseline | | header | table | | |
| | | | header | | |
| SPKMVQGSGCF | SEQ | 10.57 | 8.48 | 23.26 | 30.63 |
| GRKMDRISSSS | ID NO: | (7.90) | (6.50) | (7.15) | (4.47) |
| GLGCKVLRRH | 48 | | | | |
| CH3(CH2)₁₄(C═ | SEQ | 8.99 | 4.93 | 158.3* | 79.42*** |
| O)-Aeea-Aeea- | ID NO: | (4.07) | (0.93) | (39.91) | (5.58) |
| Aeea-Dap-Dap- | 49 | | | | |
| SPKQVQGSGCF | | | | | |
| GRKQDRISSSSG | | | | | |
| LGCKVLRRH | | | | | |
| (disulfide bond) | | | | | |
| CH3(CH2)₁₄(C═ | SEQ | 21.64 | 12.73 | 53.78** | 16.88 |
| O)-Aeea-Aeea- | ID NO: | (4.76) | (7.0) | (18.57) | (4.84) |
| Aeea-Dap-Dap- | 50 | | | | |
| SPKMVQGSGCF | | | | | |
| GRKMDRISSSS | | | | | |
| GLGCKVLRRH | | | | | |
| (disulfide bond) | | | | | |

*P < 0.05,**P < 0.01,***P < 0.001,****P < 0.0001 compared to corresponding Natriuretic Peptide: unmodified ANP, BNP, or CNP; Statistical analysis was based on Student's t test performed by GraphPad version 9.1.1; N.D . = Not determined Note: Because unmodified ANP, BNP, and CNP have a short half-life, their maximum concentration (Cmax) occurs before the 2-hour time point and is therefore not included in this table.

Example 4: Subcutaneous administration of cationic alkyl modified CNP resulted in prolonged plasma retention and enhanced bioactivity compared to native CNP.

The following experimental protocol was followed to generate data for FIG. 1. Animal care and treatment: Male CD-1 mice aged 6 to 9 weeks (Charles River, Hollister, CA) were housed in groups of 5-6 per disposable polypropylene cage with rodent cob bedding and given ad libitum access to LabDiet Pico Rodent #5053 food and water. The animals were kept in PharmaIN animal facility with controlled temperature (68-74° F.) and humidity (30-60%), and a 12-hour light/dark cycle. Drug administration: Male CD-1 mice received single subcutaneous injections of either 1.0 mg/kg of native human CNP (SEQ ID NO: 32 with sequence GLSKGCFGLKLDRIGSMSGLGC; Chempep Inc. Wellington, FL) or a cationic alkyl-modified CNP derivative (SEQ ID NO: 29 with sequence CH3 (CH2)₁₄(C═O)-Aeea-Aeea-Aeea-Dab-Dab-GLSKGCFGLKLDRIGSQSGLGC or SEQ ID NO: 31 with sequence CH3(CH2)₁₄(C═O)-Aeea-Aeea-Aeea-Dap-Dap-GLSKGCFGLKLDRIGSQSGLGC) synthesized at PharmaIN. Lead buffer was used to formulate or dissolve all test articles on the day of administration. Blood sampling: Blood samples were collected via retro-orbital bleed at several timepoints (0, 0.5, 1, 2, 4, 6, 8, and 24 hours) and processed in K₂EDTA tubes (2000× g; 15 min, 4° C. within 30 minutes after collection) to obtain plasma. Plasma samples were aliquoted and stored at −80° C. until analysis. Biochemical analysis: For pharmacokinetic study, plasma aliquots were thawed at 4° C. then analyzed using a commercially available CNP ELISA kit from Phoenix Pharmaceuticals (cat #EKE-012-03), which detects the cyclic structure of CNP and all CNP derivatives with the same level of reactivity to the ELISA kit. The CNP derivatives are modified human CNP molecules in which the methionine in the cyclic portion of native CNP (i.e., GLSKGCFGLKLDRIGSMSGLGC) was replaced by glutamine (Q) (i.e., GLSKGCFGLKLDRIGSQSGLGC) and the N-terminal was extended with either CH3(CH2)₁₄(C═O)-Aeea-Aeea-Aeea-Dab-Dab [SEQ ID NO: 10] or CH3(CH2)₁₄(C═O)-Aeea-Aeea-Aeea-Dap-Dap [SEQ ID NO: 14] for SEQ ID NO: 29 and 31, respectively. For pharmacodynamic or bioactivity study of cGMP, plasma aliquots were thawed at 4° C. then analyzed using a commercially available cGMP kit from Abcam (ab133052, Waltham, MA).

Example 5: Single and repeat administration of cationic alkyl modified C-type Natriuretic Peptides increases the probability of survival in LPS induced sepsis and ALI animal models.

Both SEQ ID NO: 31 and SEQ ID NO: 29 are effective in improving survival from ALI when administered to mice either subcutaneously (SC) or intratracheally (IT). The following experimental protocol was followed to generate data for FIG. 2. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). Drug administration for FIG. 2A): Mice were injected intraperitoneally (IP) with LPS (15 mg/kg; Sigma-Aldrich) and treated with various test articles, including cationic alkyl modified CNPs (SEQ ID NO: 31 and 29; 0.3 mg/kg (0.1 μmol/kg) SC). The control group received LPS treatment without any test article. The test articles were administered immediately after LPS administration. Survival was monitored every 2 hours from 8-56 hours, after which the mice were euthanized under isoflurane anesthesia. Statistical analysis was based on Gehan-Breslow-Wilcoxon test performed by using GraphPad Prism (n=10, 10, and 10; Control, SEQ ID NO: 31, and SEQ ID NO: 29). ** P<0.01, * P<0.05 vs Control group. Drug administration for FIG. 2B): Mice were injected intratracheally (IT) with LPS (20 mg/kg; Sigma-Aldrich) and treated with various test articles, including cationic alkyl modified CNPs (SEQ ID NO: 31 and 29; 0.3 mg/kg (0.1 μmol/kg) IT). The control group received LPS treatment without any test article. The test articles were administered immediately after LPS administration and repeated every 24 hours for a total of 3 bolus doses. Survival was monitored every 8 hours for 72 hours, after which the mice were euthanized under isoflurane anesthesia. Statistical analysis was based on Gehan-Breslow-Wilcoxon test performed by using GraphPad Prism (n=6, 6, and 6; Control, SEQ ID NO: 31, and SEQ ID NO: 29). ** P<0.01, * P<0.05 vs Control group.

Example 6: Bolus administration of cationic alkyl modified C-type Natriuretic Peptides suppresses lung injury indicating the resolution of ALI/ARDS.

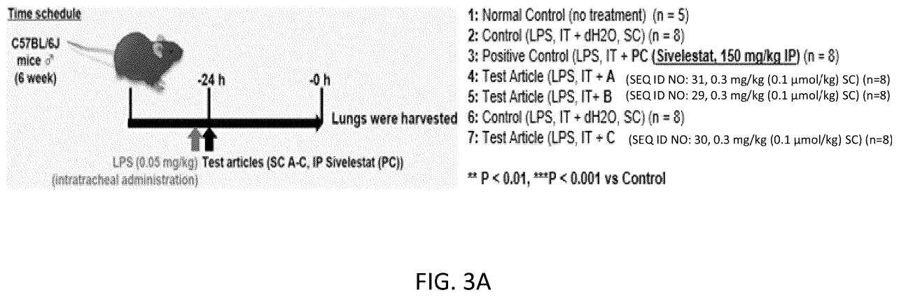

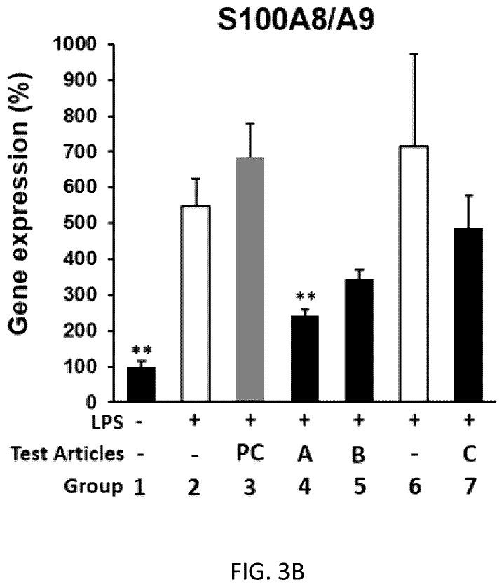

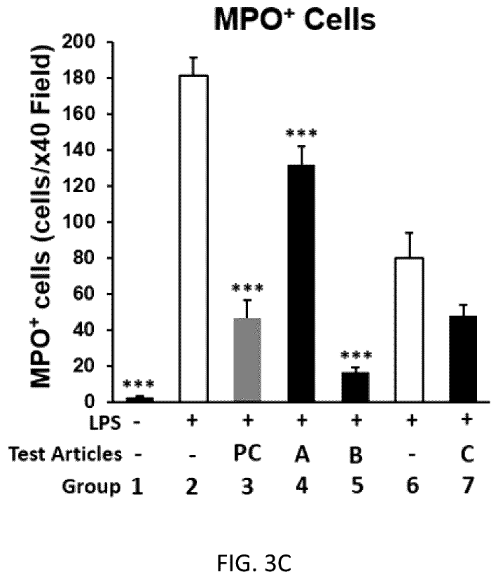

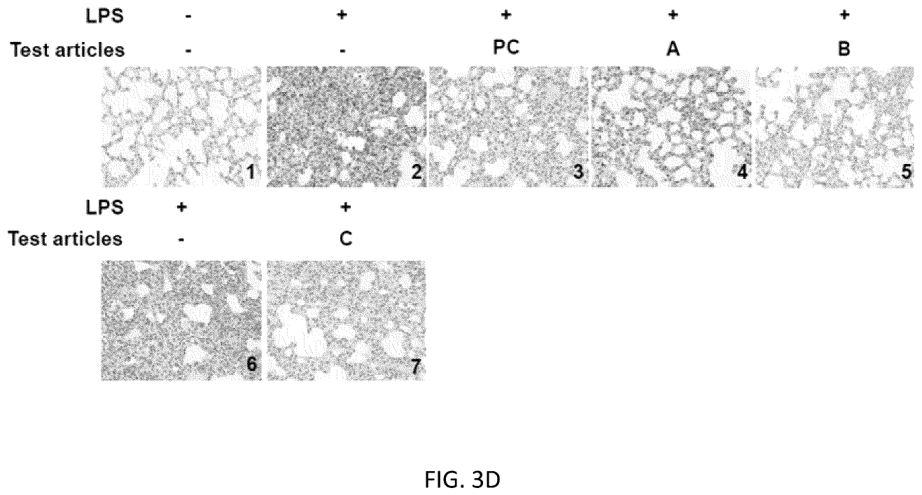

The following experimental protocol was followed to generate data for FIG. 3. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). Drug administration: Mice were administered LPS (0.05 mg/kg IT; Sigma-Aldrich) followed by treatment with various test articles, including Sivelestat (150 mg/kg; Nipro, Osaka, Japan), an inhibitor of human neutrophil elastase, injected intraperitoneally (IP) as a positive control, and cationic alkyl modified CNPs (SEQ ID NO: 31, 29, and 30; 0.3 mg/kg (0.1 μmol/kg) SC). The test articles were administered immediately after LPS injection. Additionally, a normal control (NC) group without LPS administration and a Control group receiving only LPS without any test article were included. After 24 hours, the mice were euthanized under isoflurane anesthesia and their lungs were harvested for analysis. Biochemical analysis for FIG. 3B): Lungs were minced in Tri-Reagent (Cosmo Bio, Tokyo, Japan) followed by the addition of CHCl3. After incubation at room temperature for 3 min, the samples were centrifuged (12,000×g, 4° C. for 15 min). The water layer was collected, and an equal volume of 2-propanol was added. After incubating for 10 min at room temperature, the samples were centrifuged (12,000×g, 4° C. for 15 min), and the supernatant was removed. Then, 75% EtOH was added, and the samples were centrifuged (12,000×g, 4° C. for 5 min). The supernatant was removed, and the pellets were dissolved in nuclease-free water (Ambion, MA, USA). The gene expression levels of S100A8 and S100A9 were measured with qRT-PCR-analysis by using a cDNA synthesis kit (Qiagen; Venlo, the Netherlands). Expression of myeloid cell-derived proteins (S100A8/A9) are elevated in several types of inflammatory lung disorders. Statistical analysis was based on Dunnett's test performed by using GraphPad (n=5, 8, 8, 8, 8, 8, and 8; NC, Control, PC, A [SEQ ID NO: 31], B [SEQ ID NO: 29], Control, D [SEQ ID NO: 30]). *** P<0.001, ** P<0.01, * P<0.05 vs each corresponding Control group. Biochemical analysis for FIG. 3C,D): Lung tissue was fixed by 4% paraformaldehyde. Fixed lung tissue was embedded in paraffin and sectioned.

Immunohistochemical staining was performed on the sections using anti-MPO rabbit polyclonal antibody (Agilent Technologies; Santa Clara, CA) followed by horseradish peroxidase-labeled anti-rabbit IgG goat polyclonal antibody (Nichirei Bioscience Inc.; Tokyo, Japan) and detection with 3,3′-Diaminobenzidine-4HCl (DAB) (Agilent Technologies; Santa Clara, CA). The number of myeloperoxidase positive (MPO+) cells was quantified per field of view. Increased neutrophil count is often seen in ALI and ARDS. MPO+ cells serve as a direct measure of neutrophil presence. Statistical analysis was based on Dunnett's test performed by using GraphPad (n=5, 8, 8, 8, 8, 8, and 8; NC, Control, PC, A [SEQ ID NO: 31], B [SEQ ID NO: 29]; Control, D [SEQ ID NO: 30]). *** P<0.001 vs each corresponding Control group.

Example 7: Cationic alkyl modified CNP derivatives decreased neutrophil infiltration in the lung, indicating resolution of ALI/ARDS.

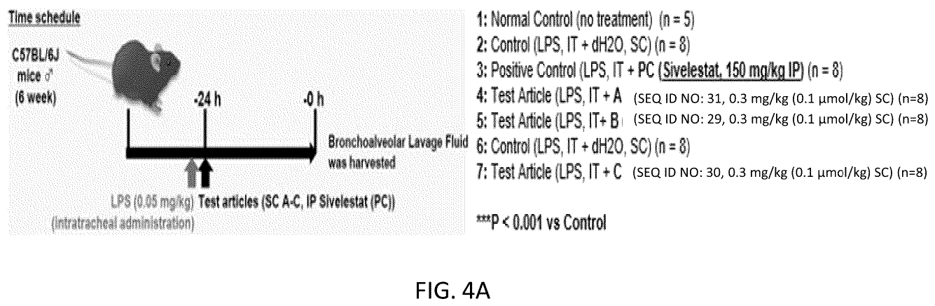

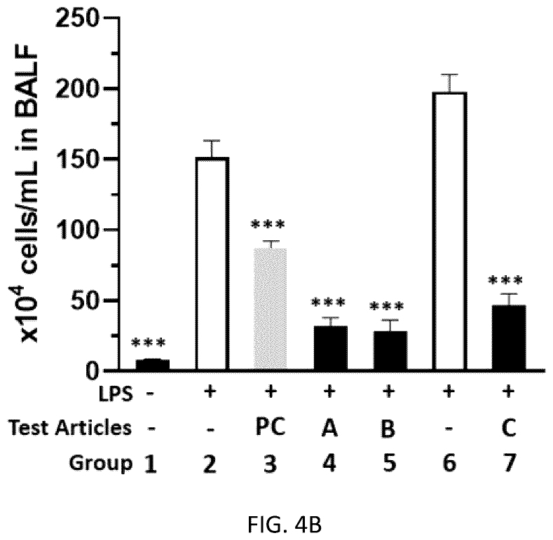

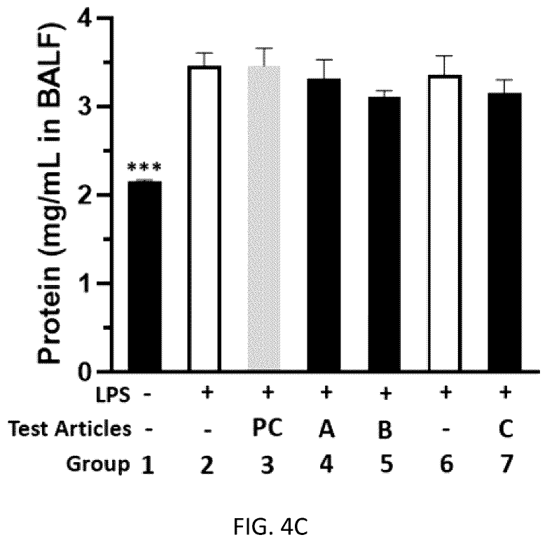

The following experimental protocol was followed to generate data for FIG. 4. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). Drug administration: Mice were administered LPS (0.05 mg/kg IT; Sigma-Aldrich) followed by treatment with various test articles, including Sivelestat (150 mg/kg; Nipro, Osaka, Japan), an inhibitor of human neutrophil elastase, injected IP as a positive control, and cationic alkyl modified CNPs (SEQ ID NO: 31, 29, and 30; 0.3 mg/kg (0.1 μmol/kg) SC). The test articles were administered immediately after LPS injection. Additionally, a normal control (NC) group without LPS administration and a Control group receiving only LPS without any test article were included. After 24 hours, the mice were euthanized under isoflurane anesthesia and bronchoalveolar lavage fluid (BALF) was harvested. Biochemical analysis: The total cell number in BALF was counted with a counting chamber. Total protein concentration in BALF was measured with PierceTM BCA Protein Assay Kit (Thermo Fisher Scientific). Statistical analysis was based on Dunnett's test performed by using GraphPad (n=5, 8, 8, 8, 8, 8, and 8; NC, Control, PC, A [SEQ ID NO: 31], B [SEQ ID NO: 29]; Control, D [SEQ ID NO: 30]). *** P<0.001, vs each corresponding Control group. ALI and ARDS are associated with increased cells in BALF, particularly neutrophils. To assess the resolution of ALI/ARDS in animal models, it is common to measure the number of cells (FIG. 4B) and total protein levels (FIG. 4C), which serve as a marker for neutrophils. A decrease in these markers indicates the resolution of ALI/ARDS.

Example 8: Table showing treatment with cationic alkyl modified CNP derivatives attenuated LPS-induced upregulation of inflammatory cytokines in BALF.

The following experimental protocol was followed to generate data for Table 4. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). Drug administration: Mice were administered LPS (0.05 mg/kg IT; Sigma-Aldrich) followed by treatment with various test articles, including Sivelestat (150 mg/kg; Nipro, Osaka, Japan), an inhibitor of human neutrophil elastase, injected IP as a positive control, and cationic alkyl modified CNPs (SEQ ID NO: 31, 29, and 30; 0.3 mg/kg (0.1 μmol/kg) SC). The test articles were administered immediately after LPS injection. Additionally, a normal control (NC) group without LPS administration and a Control group receiving only LPS without any test article were included. After 24 hours, the mice were euthanized under isoflurane anesthesia and BALF was harvested. Biochemical analysis: ALI and ARDS are often characterized by increased levels of inflammatory cytokines, including Macrophage Chemoattractant Protein-1 (MCP1), Interleukin-6 (IL-6), and Tissue Necrosis Factor α (TNFα), in BALF. Decreases in their concentration can indicate the resolution of ALI/ARDS. Concentration of each cytokine (MCP1, IL-6, and TNFα) was measured using an ELISA kit (R&D SYSTEMS, Minneapolis M N). Previous studies have highlighted the essential role of TNFα (see, e.g., PLoS One, 2014 Jul.22;9(7):e102967) and have demonstrated that non-survivors exhibit upregulation of TNFα and IL-6 (see, e.g., Chest, 1997:111:1306-21), while MCP-1 is elevated in the group who developed ALI/ARDS (see, e.g., International Journal of Molecular Sciences, 2019:20 (9): 2218). Statistical analysis was based on Dunnett's test performed by using GraphPad. (n=5, 8, 8, 8, and 8; NC, Control, PC, SEQ ID NO: 31, SEQ ID NO: 29). *** P<0.001, ** P<0.01, * P<0.05 vs each corresponding Control group.

TABLE 4
Single subcutaneous injection of SEQ. ID. NO. 31 and SEQ. ID.
NO. 29 showed attenuated LPS-induced upregulation of inflammatory
proteins in bronchoalveolar lavage fluid (BALF).

| | % Of Control | | | | |
|---|---|---|---|---|---|
| Parameter | Normal | | | SEQ. ID. | SEQ. ID. |
| tested in BALF | Control | Control | Sivelestat | NO. 31 | NO. 29 |
| TNFα | 1*** | 100 | 63 | 28** | 39** |
| IL-6 | 18*** | 100 | 69 | 56* | 76 |
| MCP1 | 6*** | 100 | 63 | 50** | 69 |

***P < 0.001,
**P < 0.01,
*P < 0.05, vs Control Group
Notes:
Increased number of inflammatory cytokines, especially tumor necrosis factor alpha (TNFα), interleukin-6 (IL-6), and monocyte chemotactic protein 1 (MCP1) are the observed characteristics of ALI and ARDS. For this study, C57BL/6J mice were treated with LPS (0.05 mg/kg IT) and treated with various test articles. These include Sivelestat (positive control (PC); inhibitor of human neutrophil elastase; 150 mg/kg IP), SEQ. ID. NO. 31 (0.3 mg/kg (0.1 μmol/kg) SC), and SEQ. ID. NO. 29 (0.3 mg/kg (0.1 μmol/kg) SC). Test articles were administered right after LPS administration. Normal control group (NC) without LPS treatment, and LPS treated groups without test article treatment (Control) were included. Twenty-four-hour post treatment, mice were sacrificed under isoflurane anesthesia and then BALF was harvested to measure previously mentioned cytokines. Statistical analysis was based on Dunnett's test performed by using GraphPad (n = 5, 8, 8, 8, and 8; NC, Control, PC, SEQ. ID. NO. 31, and SEQ. ID. NO. 29).
***P < 0.001,
**P < 0.01,
*P < 0.05 vs Control group.

Example 9: The effect of cationic alkyl modified CNP derivatives in inflammation status of acute exacerbation of idiopathic pulmonary fibrosis (IPF-AE) in the lung, indicating resolution of ALI/ARDS.

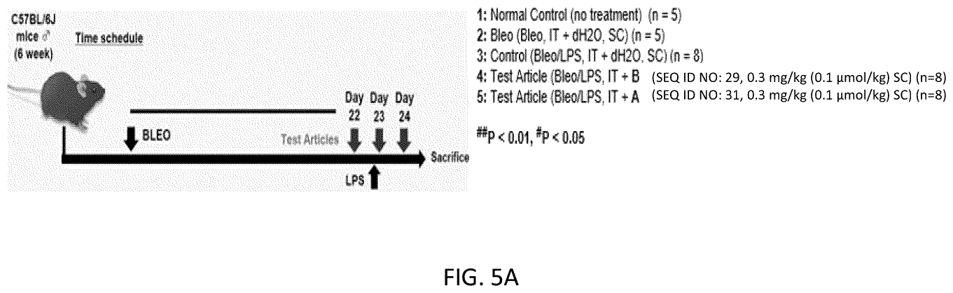

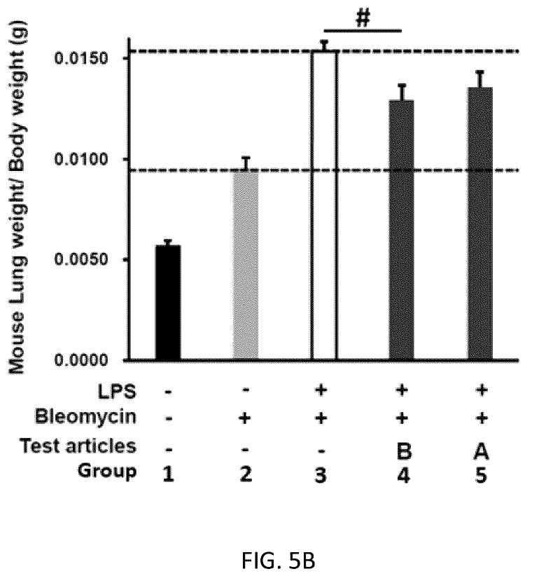

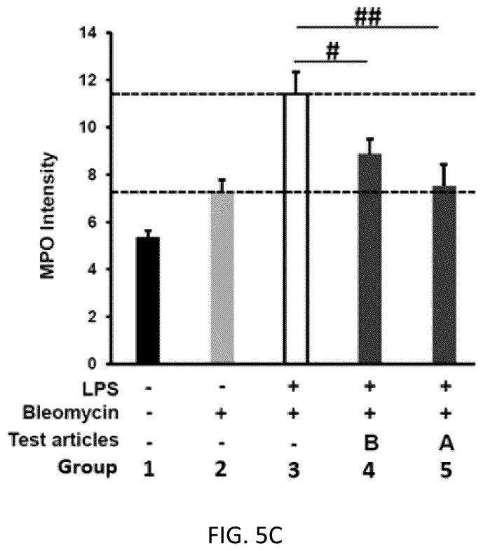

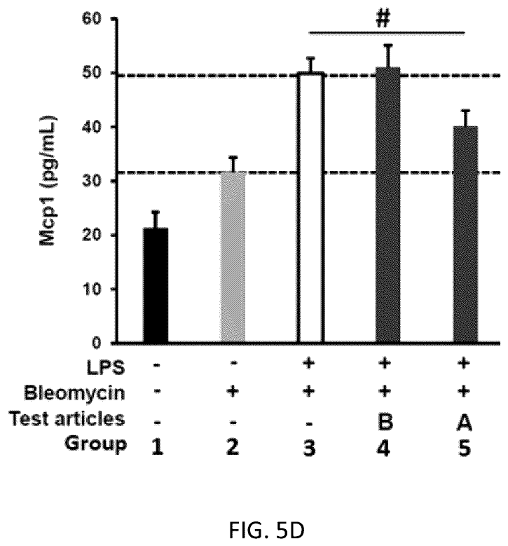

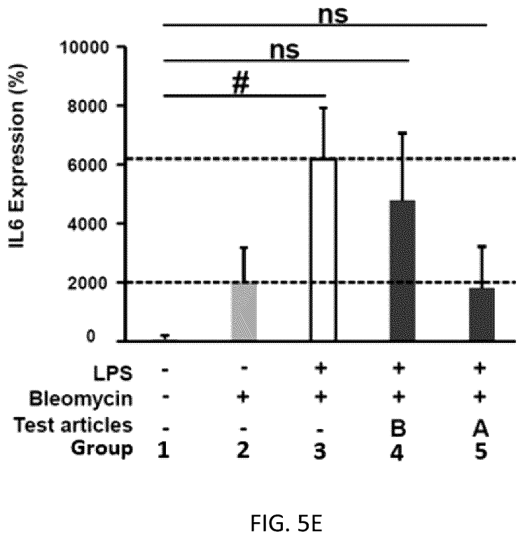

The following experimental protocol was followed to generate data for FIG. 5. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). Drug administration: Mice received IT administration of bleomycin (Bleo; 1.0 mg/kg; Nippon Kayaku Tokyo, Japan) followed by IT administration of LPS (0.025 mg/kg; Sigma Aldrich, St. Louis, MO, USA) after 3 weeks. Subcutaneous treatment at 0.3 mg/kg (0.1 μmol/kg) with either SEQ ID NO: 29 or SEQ ID NO: 31 was administered as shown in FIG. 5A. Additionally, a normal control (NC) group without LPS/Bleo treatment, a Bleo group without LPS treatment, and a Control group without test article treatment were also included. On the final day, mice were euthanized under isoflurane anesthesia, and their lung tissues were harvested and weighed (FIG. 5B). Lung weight/body weight ratio increase is a commonly measured parameter that indicates lung damage. Biochemical analysis for FIG. 5C): Lung tissue was harvested and fixed by 4% paraformaldehyde. Paraffin section of fixed lung tissue was stained immunohistochemically with anti-MPO rabbit polyclonal antibody (Agilent Technologies Santa Clara, CA), horseradish peroxidase (HRP)-labeled anti-rabbit IgG goat polyclonal antibody (Nichirei bioscience Inc. Tokyo, Japan) and 3,3′-Diaminobenzidine-4HCl (DAB) (Agilent Technologies Santa Clara, CA). The expression of MPO was assessed by Image J (NIH, Bethesda, MD, USA). Biochemical analysis for FIG. 5D): Lung tissue was minced in lysis buffer and diluted by PBS (Fujifilm, Tokyo, Japan). Macrophage chemoattractant protein-1 (MCP1) was measured by using ELISA kit (R&D SYSTEMS, Minneapolis M N). Biochemical analysis for FIG. 5E): Lungs were minced in Tri-Reagent (Cosmo Bio, Tokyo, Japan) followed by the addition of CHCl3. After incubation at room temperature for 3 min, the samples were centrifuged (12,000×g, 4° C. for 15 min). The water layer was collected, and an equal volume of 2-propanol was added. After incubating for 10 min at room temperature, the samples were centrifuged (12,000×g, 4° C. for 15 min), and the supernatant was removed. Then, 75% EtOH was added, and the samples were centrifuged (12,000×g, 4° C. for 5 min). The supernatant was removed, and the pellets were dissolved in nuclease-free water (Ambion, MA, USA). The gene expression levels of IL-6 were measured with qRT-PCR-analysis by using cDNA synthesis kit (Qiagen; Venlo, the Netherlands). Previous reports have demonstrated the upregulation of cell numbers for neutrophils (see, e.g., Kona M., et al., Respir. Med., 2021, vol. 186.), MCP1 (see, e.g., Arai T., et al., BMJ Open Respir. Res., 2021, vol. 8, 1.), and IL-6 in non-survivors (see, e.g., Lee J., et al., PloS one, 2021, vol. 16,7.). Statistical analysis was based on Student's t test performed by using GraphPad (n=5, 5, 8, 8, 8; NC, Bleo, Control, A [SEQ ID NO: 29], B [SEQ ID NO: 31]). ##P<0.01 and #P<0.05.

Example 10: Repeat subcutaneous administration of cationic alkyl modified CNP derivatives demonstrated significant anti-tumor activity in an orthotopic mouse model of breast cancer using E0771 cells.

The groups treated with cationic alkyl modified CNP (SEQ ID NOs: 29, 30, and 31) showed a significant decrease in tumor volume compared to the Control group at the conclusion of the study. A cationic alkyl sequence without CNP (SEQ ID NO: 14) was also tested in this model but did not show a significant reduction in tumor volume. Therefore, it can be concluded that the attachment of CNP is essential for anti-tumor activity. The following experimental protocol was followed to generate data for FIG. 6. Animal care and treatment: Female C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan or PicoLab Rodent Diet 20, LabDiet Corp., St. Louis, Missouri). Implantation and drug administration: Mice were implanted with E0771 breast cancer cells (250,000 cells/mouse; Cosmo Bio Tokyo, Japan) in their left mammary gland and were randomized into groups of n=10. Starting from Day 4 post-inoculation, cationic alkyl modified CNP derivatives (SEQ ID NOs: 29, 30, and 31) were administered subcutaneously at a bolus dose of 0.3 mg/kg (0.1 μmol/kg; with a dose volume of 10 mL/kg) in sterile water for injection (Otsuka Pharmaceutical, Tokushima, Japan) once daily for 5 days (5 days on, 2 days off) for 3 cycles. The control group, which received only buffer, was dosed in the same manner to establish the baseline tumor growth kinetics. Tumor sizes were measured using calipers. Statistical analysis was based on Dunnett's test performed by using GraphPad (n=10, 10, 10, and 10; Control, SEQ ID NO: 29, SEQ ID NO: 30, and SEQ ID NO: 31) **** P<0.0001 vs Control group.

Example 11: Fluorescence Polarization (FP) Assay for Natriuretic Peptide Receptor B (NPRB) and Natriuretic Peptide Receptor C (NPRC)

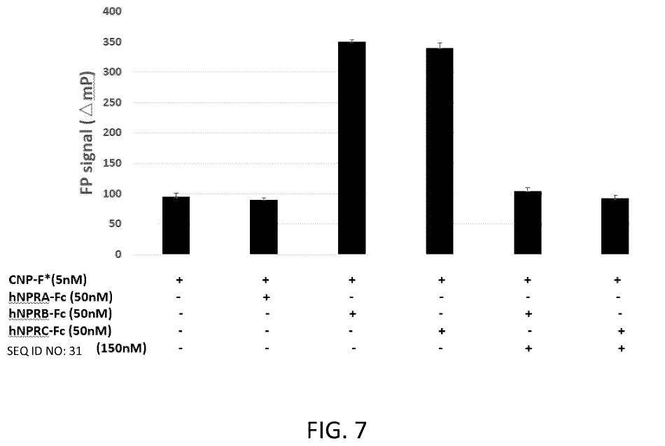

A C-type natriuretic peptide (CNP) bound to 5(6)-Carboxyfluorescein (F*) based probe alone (5 nM CNP-F*) alone has rapid rotation and results in low fluorescence polarization (FP) signal. As shown in FIG. 7, when human NPRB or NPRC (50 nM) is added, the CNP probe binds to those receptors, which results in slow rotation and high FP signal. In the presence of NPRA, no FP signal change was detected, which suggests CNP probe does not bind to NPRA. In the presence of [SEQ ID. No. 31], the low FP signal demonstrates that the CNP probe binds to both NPRB and NPRC.

### Fluorescence Polarization (FP) Assay

The CNP-F* probe was incubated at a final concentration of 5 nM in the presence of 50 nM human NPRB or NPRC in an assay buffer containing PBS, pH 7.4 and 0.01% Triton X-100. A 100-L volume of CNP-F* probe and NPR were dispensed into a black 96-well Costar flat-bottom polystyrene plate prior to FP measurement. Next, 1-L volume of SEQ ID. No. 31 (final concentration 150 nM) was added to the premixed probe and NPR. The plate was incubated at room temperature for 10 min. Fluorescence polarization was then measured on Flexstation 3 (excitation wavelength: 480 nm; emission wavelength: 525 nm and cutoff at 515 nm).

### CNP-F* Probe Synthesis.

CNP-F* was prepared using solid phase peptide synthesis. Briefly, the linear peptide (Sequence: F*-GLSKGCFGLKLDRIGSMSGLGC, F* is 5(6)-Carboxyfluorescein) was synthesized in an Automated Microwave synthesizer (CEM, Matthews, NC). Next, the crude linear peptide was cleavage with 95% TFA in the presence of carbocation scavengers and ether precipitation. The disulfide bond formation of the peptide was performed at high dilution in 10% DMSO. Finally, the produce was purified and characterized by reversed phase HPLC (1260 Infinity II Preparative LC Systems, Agilent Technologies, Santa Clara, CA) and the mass is confirmed using an LCMS system (6100 Series Single Quadrupole LC/MS, Agilent Technologies, Santa Clara, CA).

### Recombinant NPR-Fc Fusion Proteins

Recombinant human NPRA, NPRB and NPRC extracellular domains (ECD) were expressed in mammalian cells as soluble human IgG1 Fc fusions, essentially as described previously (Bennet et al, JBC 266 (34) 23060-23067, 1991.) Briefly, the mature human NPR ECD peptide sequences, NPRA N1-L439, (GenBank Accn. #XP_005245275.1), NPRB R1-T433, (GenBank Accn. #NP_003986.2) and NPRC Q1-S434, (GenBank Accn. #NP_001191304.1), were synthetically linked (Azenta, Burlington,MA) to amino acids E216-G446 (IMGT.org, EU numbering) of human IgGI heavy chain (Accn. P01857.2) and cloned into pCMV6-a-puro (Invitrogen, Carlsbad,CA) for mammalian expression. A single C>S substitution was introduced at position C232 of NPRA, an unpaired cysteine residue, to eliminate disulfide-linked aggregation (Olympic Protein Technologies, PA1 Report, unpublished). All NPR-Fc plasmid sequences were verified and plasmids produced using endotoxin-free reagents (Azenta, Burlington,MA).

Each NPR-Fc was transfected at 200 mls scale in Expi293 cells (A-14635, Thermo-Fisher Scientific). After 4 days growth, the transfection supernatants were harvested, filtered, Protein A captured (MabSelect SuRe, Cytiva) and eluted in 0.1M Citrate buffer, pH 6.0/3.5 mM MgCl2/2% glycerol, for NPRA-Fc and NPRC-Fc. NPRB-Fc was eluted at pH 6.6 (Pierce Gentle Elution buffer, Thermo Fisher catalog #21027). SDS-PAGE analysis showed bands of 80-85 kDa bands under reducing conditions, larger than the expected 75 kDa bands most likely due to glycosylation in the ECDs of all 3 NPRs (Potter L R, et al. Handb Exp Pharmacol. 2009;(191):341-366). Analytic SEC confirmed the main peaks running larger than the 153 kDa MW marker. Preparative SEC removed the HMW protein from both NPRA and NPRC-Fc and purified them both to 93% monomer followed by buffer exchange into PBS. NPRB-Fc at 98% monomer required no further purification. (Olympic Protein Technologies, PA1 Report, unpublished).

Example 12: The effect of cationic alkyl modified CNP derivative alone or in combination with pirfenidone indicates reduction in lung fibrosis (inversely proportional to alveolar area) in mouse model of idiopathic pulmonary fibrosis (IPF).

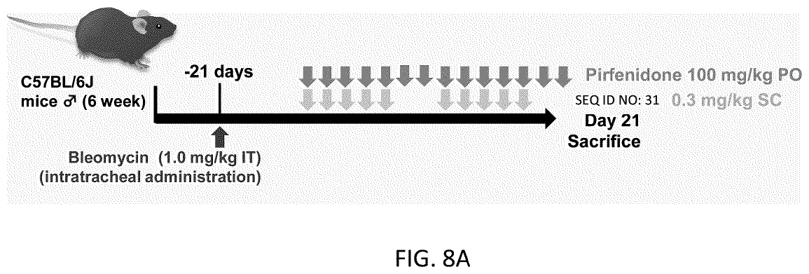

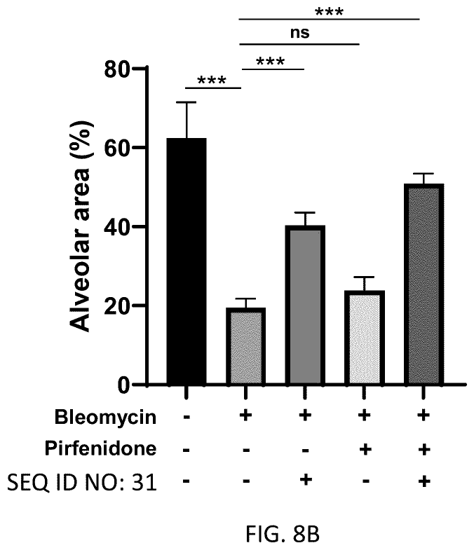

The following experimental protocol was followed to generate data for FIGS. 8A and 8B. Animal care and treatment: Male C57BL/6J mice (6 weeks-old) were purchased from Kyudo (Saga, Japan) and maintained under a 12-hour light/12-hour dark cycle with free access to water and standard mouse diet (MF diet, Oriental Yeast Co., Ltd. Tokyo, Japan). To induce lung fibrosis, mice received IT administration of bleomycin (Bleo; 1.0 mg/kg; Nippon Kayaku Tokyo, Japan). Drug administration started after 7 days: SEQ ID NO: 31 (SC at 0.3 mg/kg (0.1 μmol/kg)), pirfenidone (PO at 100 mg/kg), or combination of pirfenidone (Pir; PO at 100 mg/kg) and SEQ ID NO: 31 (SC at 0.3 mg/kg (0.1 μmol/kg)) were administered as shown in FIG. 8A. Additionally, a normal control (NC) group without Bleo treatment and a Bleo Control group without test article treatment were also included in this study. On Day 21, mice were euthanized under isoflurane anesthesia, and their lung tissues were harvested. Biochemical analysis for FIG. 8B): Lung tissue was harvested and fixed by 4% paraformaldehyde, and Azan staining was performed by Kyushu University (Fukuoka, Japan). Alveolar area, which is inversely proportional to fibrotic area, were measured by Image J in a blinded setting (NIH, Bethesda, MD, USA). Statistical analysis was based on Student's t test performed by using GraphPad (n=3, 7, 7, 7, and 7; NC, Bleo Control, SEQ ID NO: 31, Pir, and Combo (SEQ ID NO: 31 & Pir). *** P<0.001, ns=not significant vs NC. Groups treated with SEQ ID NO: 31 alone or in combination with pirfenidone showed a significantly higher alveolar area, which demonstrates the presence of healthy tissue and a reduction in fibrosis.

Example 13: Repeat subcutaneous administration of cationic alkyl modified CNP derivative demonstrated significant anti-tumor activity as a monotherapy or in combination with an immune checkpoint inhibitor in an orthotopic mouse model of breast cancer using E0771 cells.

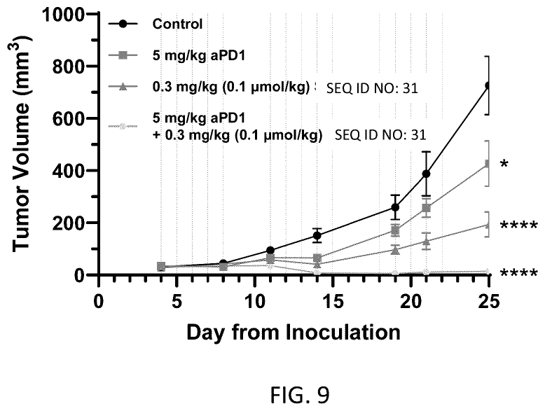

The following experimental protocol was followed to generate data for FIG. 9. Animal care and treatment: Female C57BL/6J mice (6 weeks-old) were purchased from Kyudo (Saga, Japan) and maintained under a 12-hour light/12-hour dark cycle with free access to water and standard mouse diet (CRF diet; Oriental Yeast Co., Ltd, Tokyo Japan). Implantation and drug administration: Mice were orthotopically implanted with E0771 breast cancer cells (250,000 cells/mouse; Cosmo Bio Tokyo, Japan) in their left mammary gland and were randomized into groups of n=7-8. Drug administration started from Day 4 post-inoculation, anti-PD1 Ab (aPD1; BioX cell, West Lebanon, NH, #RMP1-14) was administered intraperitoneally at 5 mg/kg twice a week for 2 cycles; SEQ ID NO: 31 was administered subcutaneously at a bolus dose of 0.3 mg/kg (0.1 μmol/kg) in sterile water for injection (Otsuka Pharmaceutical, Tokushima, Japan) once daily for 5 days (5 days on, 2 days off) for 3 cycles; or a combination of aPD1 (5 mg/kg IP twice a week; 2 cycles) and SEQ ID NO: 31 (0.3 mg/kg SC once a day for 5 days on and 2 days off; 3 cycles). The control group, which received only sterile water for injection, was dosed in the same manner as SEQ ID NO: 31 to establish the baseline tumor growth kinetics. Tumor sizes were measured using calipers. Statistical analysis was based on Dunnett's test performed by using GraphPad (n=8, 7, 8, and 7; Control, aPD1, SEQ ID NO: 31, Combo (aPD1 and SEQ ID NO: 31) **** P<0.0001, * P<0.05 vs Control. At the conclusion of this study, the groups treated with cationic alkyl modified CNP (SEQ ID NO: 31) showed a significant decrease in tumor volume compared to the Control group and the group treated solely with a PD1, an immune check point inhibitor.

Example 14: The combination of radiation, an immune checkpoint inhibitor, and repeat subcutaneous administration of cationic alkyl modified CNP derivative demonstrated a reduction in bone metastasis and significantly improved overall survival in orthotopic mouse model of bone metastasis using breast cancer E0771 cells.

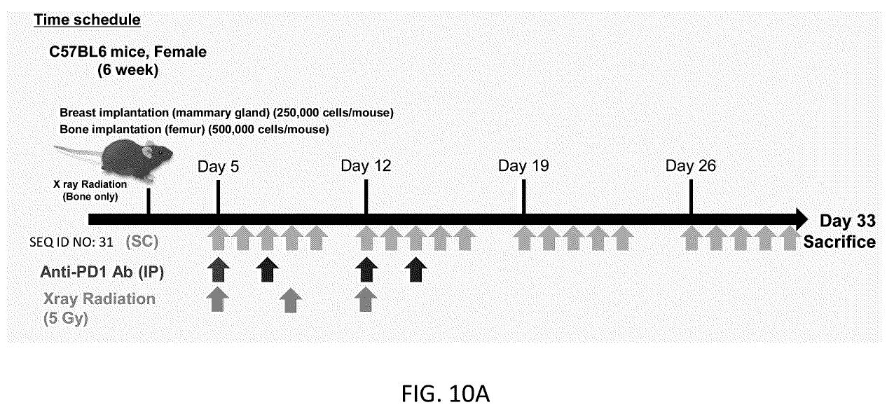

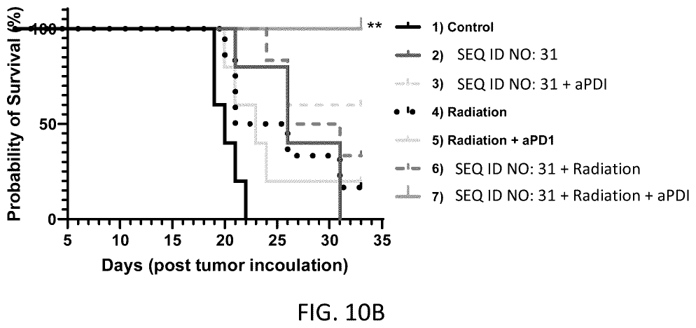

The following experimental protocol was followed to generate data for FIG. 10. Animal care and treatment: Female C57BL/6J mice (6 weeks-old) were purchased from Oriental Bioservice (Kobe, Japan) and maintained under a 12-hour light/12-hour dark cycle with free access to water and standard mouse diet (CRF diet; Oriental Yeast Co., Ltd, Tokyo Japan). Implantation and drug administration: Mice were orthotopically implanted with E0771 breast cancer cells (Cosmo Bio Tokyo, Japan; 250,000 cells/mouse in RPMI1640 medium (Fujifilm, Tokyo Japan)) in their left mammary gland and E0771 mouse breast cancer cells (500,000 cells/mouse in 50% Matrigel (Corning, NY, USA, #354234)) in their femur. Mice were then randomized into several treatment groups: 1) control (n=5), 2) SEQ ID NO: 31 (n=5), 3) SEQ ID NO: 31 and aPD1 (n=5), 4) radiation (n=6), 5) radiation and aPD1 (n=5), 6) SEQ ID NO: 31 and radiation (n=6), 7) SEQ ID NO: 31, radiation, and aPD1 (n=6). FIG. 10A. illustrates the drug administration scheme that started from Day 5 post-inoculation, SEQ ID NO: 31 was administered subcutaneously at a bolus dose of 0.3 mg/kg (0.1 μmol/kg) in buffer (15 mM succinate (TCI, Tokyo, Japan, #S0100), 4% (w/v) D-mannitol (TCI, Tokyo, Japan, #M0044), 10 mM hydoxypropyl-beta-cyclodextrin (TCI, Tokyo, Japan, #H0979) pH 4.4) once daily for 5 days (5 days on, 2 days off) for 4 cycles; aPD1 (BioX cell, West Lebanon, NH, #RMP1-14) was administered intraperitoneally at 5 mg/kg twice a week for 2 cycles; on Day 5, Day 8, and Day 12, mice were irradiated with 5 Gy of radiation in three sets at the bone by X-ray radiation system (mediXtec Japan Coporation, Japan, Chiba, MX-160Labo) under anesthesia by three types of mixed anesthetic agents (0.3 mg/kg medetomidine, 4.0 mg/kg midazolam and 5.0 mg/kg butorphanol (M/M/B: 0.3/4/5 SC; Oriental Bioservice, Kobe, Japan)). The control group, which received only buffer, was dosed in the same manner as SEQ ID NO: 31 to establish the baseline tumor growth kinetics. Survival was monitored up to Day 33 (FIG. 10B) post cell inoculation, and following scheduled sacrifice of the remaining mice, tumors were harvested, and sizes were measured using calipers. Statistical analysis was based on Log-rank (Mantel-Cox) test performed by using GraphPad (Groups: 1) control (n=5), 2) SEQ ID NO: 31 (n=5), 3) SEQ ID NO: 31 and aPD1 (n=5), 4) radiation (n=6), 5) radiation and aPD1 (n=5), 6) SEQ ID NO: 31 and radiation (n=6), 7) SEQ ID NO: 31, radiation, and aPD1 (n=6)) ** P<0.01 vs Group 5. At the conclusion of this study the combination of SEQ ID NO: 31, radiation, and aPD1 (immune checkpoint inhibitor) demonstrated a significant improvement in survival. Furthermore, the addition of SEQ ID NO: 31 reduced the incidence of bone metastasis in all its respective groups.

Example 15: Amputation in combination with repeat subcutaneous administration of cationic alkyl modified CNP derivative demonstrated a significant reduction of lung metastasis compared to amputation alone in an orthotopic mouse model of lung metastasis using osteosarcoma LM8 cells.

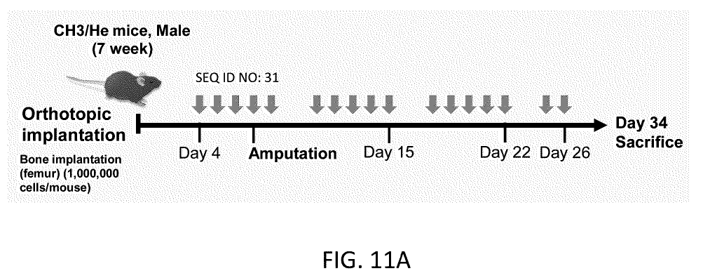

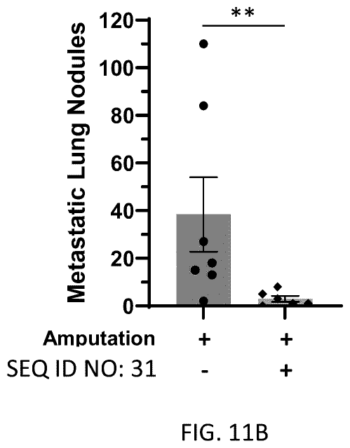

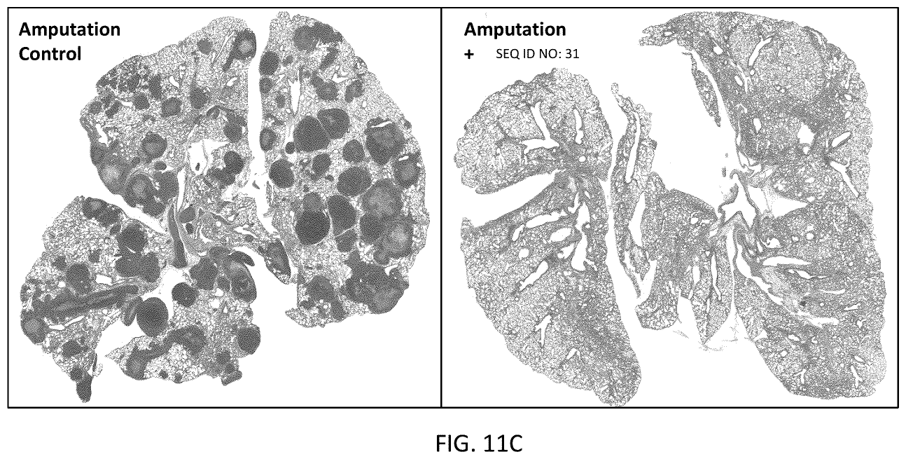

The following experimental protocol was followed to generate data for FIGS. 11A-11C. Animal care and treatment: Male CH3/He mice (7 weeks-old) were purchased from Kyudo (Saga, Japan) and maintained under a 12-hour light/12-hour dark cycle with free access to water and standard mouse diet (MF diet; Oriental Yeast Co., Ltd., Tokyo, Japan). Implantation and drug administration: Mice were orthotopically implanted with LM8 osteosarcoma cells (RCB, Tsukuba, Japan, #RCB1450) into their femur (1,000,000 cells/mouse). Mice were then randomized into groups of n=7. FIG. 11A. illustrates the drug administration scheme that started from Day 4 post-inoculation, SEQ ID NO: 31 was administered subcutaneously at a bolus dose of 0.3 mg/kg (0.1 μmol/kg) in buffer (15 mM succinate (TCI, Tokyo, Japan, #S0100), 4% (w/v) D-mannitol (TCI, Tokyo, Japan, #M0044), 10 mM hydoxypropyl-beta-cyclodextrin (TCI, Tokyo, Japan, #H0979) pH 4.4) once daily for 5 days (5 days on, 2 days off) for a little over 3 cycles. On Day 7 post-inoculation, all mice underwent amputation to remove the primary tumor under isoflurane anesthesia and were sutured with suture thread (Alfresa Pharma Corporation, Osaka, Japan, #HR0806NW45-KF2). In place of treatment, the amputated control group also received buffer once daily for 5 days (5 days on, 2 days off) for a little over 3 cycles. Mice were sacrificed on Day 34, and lung tissues were harvested. Biochemical analysis for FIG. 11B) Harvested lung tissues were soaked in 4% paraformaldehyde (Fujifilm, Tokyo, Japan, #163-20145). The tissues were then paraffin-embedded, and sections obtained were stained with hematoxylin and eosin (H&E) by Kyodo Byori (Kobe, Japan). Lung images were observed using the high-resolution microscopy (Keyence, Tokyo, Japan, #BZ-X700), and lung metastasis was evaluated by the direct count of present metastatic nodules (FIG. 11C). Outliers were identified using the ROUT test (Q=1%). All statistical analysis was based on Dunnett's test performed by using GraphPad (n=7 and 7; Control, Combo (Amputation and SEQ ID NO: 31)) ** P<0.01 vs Control group. Along with chemotherapy, surgical interventions such as amputation may be used as prevention of lung metastasis. At the conclusion of this study, repeat SC dose of SEQ ID NO: 31 with amputation reduced the incidence of lung metastasis compared to amputation alone.

Example 16: Repeat subcutaneous administration of cationic alkyl modified CNP derivative demonstrated a significant reduction in tumor volume as a monotherapy and in combination with an immune checkpoint inhibitor in a subcutaneous mouse model of colon cancer using MC38 cells.

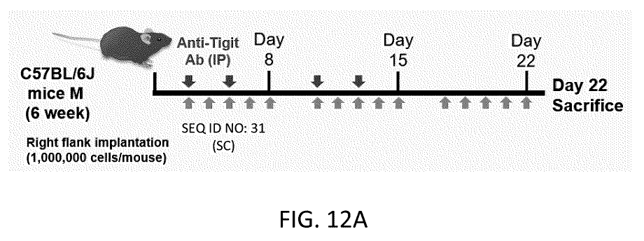

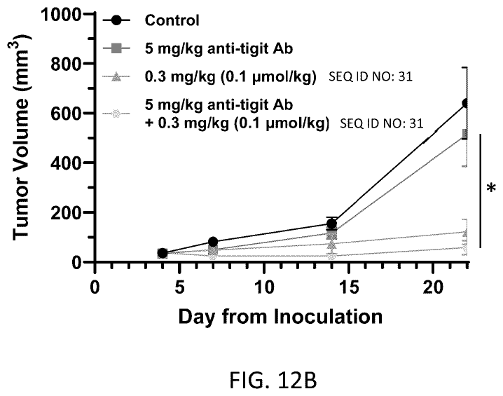

The following experimental protocol was followed to generate data for FIG. 12. Animal care and treatment: Male C57BL/6J mice (6 weeks-old) were purchased from Kyudo (Saga, Japan) and maintained under a 12-hour light/12-hour dark cycle with free access to water and standard mouse diet (MF diet; Oriental Yeast Co., Ltd, Tokyo Japan). Implantation and drug administration: Mice were subcutaneously implanted with MC38 colon cancer cells (1,000,000 cells/mouse) in their right flank and were randomized into groups of n=8-9. Drug administration started from Day 4 post-inoculation, anti-tigit Ab (Absolute antibody, Shirley, MA, USA, #Ab01258-1.1-VXX, clone 1B4) was administered intraperitoneally at 5 mg/kg twice a week for 2 cycles; SEQ ID NO: 31 was administered subcutaneously at a bolus dose of 0.3 mg/kg (0.1 μmol/kg) in buffer (15 mM succinate (TCI, Tokyo, Japan, #S0100), 4% (w/v) D-mannitol (TCI, Tokyo, Japan, #M0044), 10 mM hydoxypropyl-beta-cyclodextrin (TCI, Tokyo, Japan, #H0979) pH 4.5) once daily for 5 days (5 days on, 2 days off) for 3 cycles; or a combination of anti-tigit Ab (5 mg/kg IP twice a week; 2 cycles) and SEQ ID NO: 31 (0.3 mg/kg SC once a day for 5 days on and 2 days off; 3 cycles). The control group, which received only buffer, was dosed in the same manner as SEQ ID NO: 31 to establish the baseline tumor growth kinetics. Tumor sizes were measured using calipers on Days 4, 7, 14, and Day 22 post-inoculation. Following final tumor measurement on Day 22, mice were humanely euthanized. Statistical analysis was based on Dunnett's test performed by using GraphPad (n=9, 8, 8, and 8; Control, anti-tigit Ab, SEQ ID NO: 31, Combo (anti-tigit Ab and SEQ ID NO: 31) * P<0.05 vs anti-tigit Ab monotherapy group. At the conclusion of this study, the groups treated with cationic alkyl modified CNP (SEQ ID NO: 31) showed a significant decrease in tumor volume compared to the Control group and the group treated solely with anti-tigit Ab, an immune check point.

Example 17: HeLa cells treated with cationic alkyl modified CNP derivative demonstrated a significant inhibition of baseline cyclic adenosine monophosphate levels.

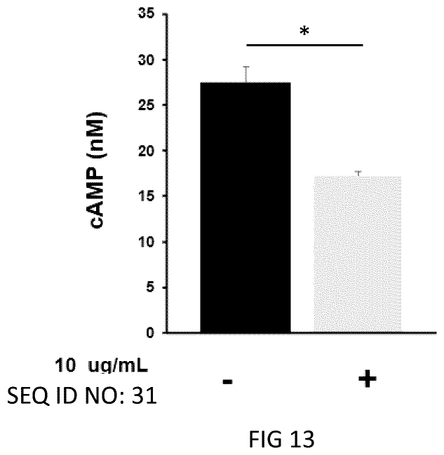

The following experimental protocol was followed to generate data for FIG. 13. In vitro protocol: HeLa cells were purchased from ATCC (Manassas, VA, USA). The cells were cultured in Dulbecco modified Eagle medium (DMEM) (Fujifilm, Tokyo, Japan, #044-29765) supplemented with 10% FBS (Sigma Aldrich, St. Louis, MO, USA, #F2442) under 100% humidity and 5% CO2 at 37° C. The cells were harvested and suspended at a concentration of 10⁷ cells/mL in ENGS (Lifeline, San Diego, CA, USA, #LEC-LL0002) and 5 uL of the cell suspension was added to each well of a 96-well low-adhesion plate (PerkinElmer, Waltham, MA, USA, #66PL96005). Then, the cells (n=4 wells) were treated with 5 uL of SEQ ID NO: 31 (final concentration of 10 μg/mL) in ENGS with 1 mM IBMX (Fujifilm, Tokyo, Japan, #099-03411) for 10 minutes. cAMP levels were evaluated using a cAMP assay kit (PerkinElmer, Waltham, MA, USA, #62AM4PEB) in accordance with its manufacture's protocol with a plate reader (PerkinElmer, Waltham, MA, USA, #Nivo). Statistical analysis was performed with GraphPad Prism against untreated control wells (n=4 wells) * P<0.05. The NPR-C receptor has been implicated in inhibiting adenylate cyclase activity, which would lead to a decrease in cyclic adenosine monophosphate (cAMP) production. The conclusion of this study resulted in changes (inhibition) of cAMP baseline levels of HeLa cells (known to express NPR-C) treated with SEQ ID NO: 31, which indicates binding to NPR-C.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound comprising a cationic alkyl moiety of Formula (I):
J-(CH2)x(CO)-(A)y-(B)z- (I),
wherein:
J is either HOOC or CH3;
x is 10-16;
A is independently selected from the group consisting of: 2-[2-(2-Aminoethoxy)ethoxy]acetic Acid (Aeea), gamma amino butyric acid (γAbu), gamma linked glutamate (γE), and alpha linked glutamate (E);
y is 2-4;
B is independently Diamino propionic acid (Dap) or Diamino butanoic acid (Dab); and
z is 2-4;
wherein -(B)z- comprises no more than 2 Dab residues and wherein the Dap or Dab residues are linked through the alpha amino.

2. The compound of claim 1, wherein:
J is CH3;
x is 10, 12, 14, or 16;
A is independently selected from the group consisting of: 2-[2-(2-Aminoethoxy)ethoxy]acetic Acid (Aeea), and gamma amino butyric acid (γAbu),
y is 3;
B is independently Diamino propionic acid (Dap) or Diamino butanoic acid (Dab); and
z is 2 or 3.

3. The compound of claim 1, wherein:
J is CH3;
x is 10, 12, 14, or 16;
A is independently selected from the group consisting of: 2-[2-(2-Aminoethoxy)ethoxy]acetic Acid (Aeea), and gamma linked glutamate (γE);
y is 3;
B is independently Diamino propionic acid (Dap) or Diamino butanoic acid (Dab); and z is 2 or 3.

4. The compound of claim 1, wherein:
J is CH3;
x is 10, 12, 14, or 16;
A is 2-[2-(2-Aminoethoxy)ethoxy]acetic Acid (Aeea),
y is 3;
B is independently Diamino propionic acid (Dap) or Diamino butanoic acid (Dab); and
z is 2 or 3.

5. The compound of claim 1, wherein:
J is CH3;
x is 10, 12, 14, or 16;
A is gamma amino butyric acid (γAbu),
y is 3;
B is independently Diamino propionic acid (Dap) or Diamino butanoic acid (Dab); and
z is 2 or 3.

6. The compound of claim 1, wherein:
J is HOOC;
x is 10, 12, 14, or 16;
A is independently selected from the group consisting of: 2-[2-(2-Aminoethoxy)ethoxy]acetic Acid (Aeea), and gamma amino butyric acid (γAbu),
y is 3;
B is independently Diamino propionic acid (Dap) or Diamino butanoic acid (Dab); and
z is 2 or 3.

7. The compound of claim 1, wherein:
J is HOOC;
x is 10, 12, 14, or 16;
A is independently selected from the group consisting of: 2-[2-(2-Aminoethoxy)ethoxy]acetic Acid (Aeea), and gamma linked glutamate (γE),
y is 3;
B is independently Diamino propionic acid (Dap) or Diamino butanoic acid (Dab); and
z is 2 or 3.

8. The compound of claim 1, wherein:
J is HOOC;
x is 10, 12, 14, or 16;
A is 2-[2-(2-Aminoethoxy)ethoxy]acetic Acid (Aeea),
y is 3;
B is independently Diamino propionic acid (Dap) or Diamino butanoic acid (Dab); and
z is 2 or 3.

9. The compound of claim 1, wherein:
J is HOOC;
x is 10, 12, 14, or 16;
A is gamma linked glutamate (γE),
y is 3;
B is independently Diamino propionic acid (Dap) or Diamino butanoic acid (Dab); and
z is 2 or 3.

10. The compound of claim 1, wherein:
J is either CH3;
x is 14;
(A)y is Aeea-Aeea-Aeea, γAbu-γAbu-γAbu, γAbu-Aeea-Aeea, γE-Aeea-Aeea, E-Aeea-Aeea; Aeea-Aeea, γAbu-γAbu, γAbu-Aeea, γE-Aeea, or E-Aeea; and
(B)z is Dap-Dap, Dab-Dab, Dab-Dap, or Dap-Dab.

11. The compound of claim 1, wherein:
J is either CH3;
x is 14;
(A)y is Aeea-Aeea-Aeea, γAbu-γAbu-γAbu, γAbu-Aeea-Aeea, γE-Aeea-Aeea, or E-Aeea-Aeea; and
(B)z is Dap-Dap, Dab-Dab, Dab-Dap, or Dap-Dab.

12. The compound of claim 1, wherein the cationic alkyl moiety is selected from SEQ ID NOs: 10 to 22 and 51 to 69.

13. The compound of claim 1, wherein the cationic alkyl moiety is selected from SEQ ID NOs: 10 to 22.

14. The compound of claim 1, wherein the compound, when conjugated to a peptide, has no clinically observable ataxia after parenteral bolus administration in rats at a dose of 10 μmol/kg and lower.

15. The compound of claim 1, wherein the compound is conjugated covalently to a peptide.

16. A conjugated peptide of Formula (II):
CH3(CH2)x(CO)-(A)y-(B)z-Peptide (II)
wherein:
x is 10-16;
A is independently selected from the group consisting of: 2-[2-(2-Aminoethoxy)ethoxy]acetic Acid (Aeea), gamma amino butyric acid (γAbu), gamma linked glutamate (7E), and alpha linked glutamate (E);
y is 2-4;
B is independently Diamino propionic acid (Dap) or Diamino butanoic acid (Dab); and
z is 2-4;
wherein:
(B)z- comprises no more than 2 Dab residues and wherein the Dap or Dab residues are linked through the alpha amino;
CH3(CH2)x(CO)-(A)y-(B)z- is covalently linked to the N-terminus of Peptide or linked to one of the side chain amino groups of Peptide;
the conjugated peptide has biological activity that is equivalent or higher than the unmodified peptide at an equivalent bolus dose; and/or
the conjugated peptide has an equivalent or higher blood level than the unconjugated peptide at the same time-point after a bolus administration at an equivalent dose.

17. The conjugated peptide of claim 16, wherein the conjugated peptide binds to a natriuretic peptide receptor and has no adverse effect or ataxia at a bolus dose of 3.0 μmol/kg and lower in rats.

18. The conjugated peptide of claim 16, wherein the CH3(CH2)x(CO)-(A)y-(B)z- moiety is covalently linked to the N-terminus of Peptide.

19. The conjugated peptide of claim 16, wherein Peptide is a natriuretic peptide of SEQ ID NO: 32, 44, 48, or 75, or a natriuretic peptide derivative.

20. The conjugated peptide of claim 16, wherein the CH3(CH2)x(CO)-(A)y-(B)z- moiety is selected from SEQ ID NOs: 10 to 22 and 51 to 69.

21. The conjugated peptide of claim 20, wherein the CH3(CH2)x(CO)-(A)y-(B)z- moiety is selected from SEQ ID NOs: 10 to 22.

22. The conjugated peptide of claim 16, wherein Peptide is a natriuretic peptide derivative with one or more methionine residues replaced by glutamine (Q), Leucine (L), Norleucine (Nle), or methoxinine (Mox).

23. The conjugated peptide of claim 16, wherein Peptide is a natriuretic peptide according to SEQ ID NO: 32, or a derivative thereof wherein one or more methionine residues are replaced by glutamine (Q), and the CH3(CH2)x(CO)-(A)y-(B)z- moiety is selected from SEQ ID NOs: 10 to 22.

24. The conjugated peptide of claim 16, wherein the conjugated peptide is selected from SEQ ID NOs: 29-31, 33-43, 45-47, 49-51.

25. The conjugated peptide of claim 24, wherein the conjugated peptide is selected from SEQ ID NOs: 29-31 and 33-43.

26. The conjugated peptide of claim 25, wherein the conjugated peptide is selected from SEQ ID NOs: 29-31.

27. The conjugated peptide of claim 26, wherein the conjugated peptide is SEQ ID NO: 29.

28. The conjugated peptide of claim 26, wherein the conjugated peptide is SEQ ID NO: 30.

29. The conjugated peptide of claim 26, wherein the conjugated peptide of Formula (II) is SEQ ID NO: 31.

30. The conjugated peptide of claim 16, wherein the conjugated peptide binds to natriuretic peptide receptor B (NPRB), natriuretic peptide receptor C (NPRC), or a combination thereof.

31. The conjugated peptide of claim 16, wherein the conjugated peptide is a NPRB agonist.

32. The conjugated peptide of claim 16, wherein the conjugated peptide is a NPRC agonist.

33. The conjugated peptide of claim 16, wherein the conjugated peptide generates a physiological effect selected from: prolonged increase blood cGMP, changes in cAMP, changes in blood pressure, increased survival from Sepsis, increased survival from Acute Lung Injury, increase survival from Acute Respiratory Distress Syndrome, decrease in MPO positive cells, decrease in number of cells in Alveolar Fluid or in Bronchoalveolar Lavage Fluid, decrease in amount of protein in Alveolar Fluid or in Bronchoalveolar Lavage fluid, decrease endothelial permeability, decrease in lung weight per body weight, decrease in Monocyte Chemoattractant Protein-1, decrease in IL-6, decrease TNF-alpha, decrease in A1008/A9, decrease fibrosis, decrease in tumor volume, decrease metastasis, decrease inflammation, antiproliferative effects, decrease cancer burden, inhibition of cyclooxygenase 2 (COX-2) expression, antagonizing the renin-angiotensin-aldosterone system, inhibiting cardiac hypertrophy, or a combination thereof.

34.-36. (canceled)

37. A method of treating a disease or condition in a subject in need thereof, comprising administering to the subject a compound of claim 1.

38. The method of claim 37, wherein the compound is selected from:
a) any one of SEQ ID NOS: 29-31, 33-43, 45-47, 49-51, or
b) any one of SEQ ID NOS: 29-31, 33-43, 45-47, or
c) any one of SEQ ID NOS: 29-31, 33-43, or
d) any one of SEQ ID NOS: 29-31, or
e) SEQ ID NO: 29 or
f) SEQ ID NO: 30, or
g) SEQ ID NO: 31.

39. The method of claim 37, wherein the compound comprises SEQ ID NO: 31.

40. The method of claim 37, wherein the disease or condition affects the lung (e.g., ALI, ARDS, COVID, inflammation, sepsis, fibrosis, or cancer), liver (e.g., non-alcoholic fatty liver disease (NAFLD), non-alcoholic steatohepatitis (NASH), inflammation, fibrosis, or cancer), heart (e.g., heart failure with preserved ejection fraction (HFpEF), heart failure with reduced ejection fraction (HFrEF), acute heart failure, or congestive heart failure), bone/joint (e.g., osteoporosis, osteoarthritis, rheumatoid arthritis, inflammation, cancer, or dwarfism), kidney (e.g., chronic kidney disease (CKD), acute kidney injury (AKI), drug induced kidney injury, inflammation/nephritis, kidney fibrosis, glomerulosclerosis, or kidney cancer), prostate (e.g., prostate hyperplasia or prostate cancer), brain, eye, skin, muscle, blood, gastrointestinal track, bladder, testis, ovary, uterus, and/or blood vessel.

41. The method of claim 37, wherein the disease or condition is pre-metastatic or post-metastatic cancer.

42. The method of claim 41, wherein the cancer is a cancer of any one or more organs selected from lung, lung pleura, liver, heart, bone/joint, kidney, prostate, breast, brain, eye, skin, muscle, blood, blood vessels, gastrointestinal track, bladder, testis, ovary, and/or uterus.

43. The method of claim 37, wherein the disease or condition is pneumonia, acute lung injury (ALI), acute respiratory distress syndrome (ARDS), or COVID in a subject in need thereof.

44. The method of claim 37, wherein the disease or condition is fibrosis.

45. The method of claim 37, wherein the treating comprises administering to the subject a therapeutically effective bolus dose of 10.0 μmol/kg or lower, and/or between 10.0 μmol/kg and 0.0001 μmol/kg inclusive.

46. The method of claim 37, wherein the compound is administered to the subject either as a monotherapy or in combination with one or more additional agents or treatments.

47. The method of claim 46, wherein when the one or more additional agents or treatments are selected from immune check point inhibitors, surgery/amputation, radiation, chemotherapy, or a combination thereof.

48. The method of claim 37, wherein the compound is administered subcutaneously, by infusion, by inhalation, by nasal spray, orally, in eye drops, or by topical application.

49. (canceled)

50. A composition comprising the compound of claim 1, and one or more pharmaceutically acceptable carriers or excipients.

51. The composition of claim 50, wherein the one or more pharmaceutically acceptable carriers or excipients comprises a bulking agent, a buffering agent, a stabilizer, a preservative, or a combination thereof.
